(19) United States
(12) Patent Application Publication
(10) Pub. No.: US 2022/0135673 A1
(43) Pub. Date: May 5, 2022

(54) METHODS AND COMPOSITIONS FOR TREATING MAST CELL GASTRITIS, MAST CELL ESOPHAGITIS, MAST CELL ENTERITIS, MAST CELL DUODENITIS, AND/OR MAST CELL GASTROENTERITIS

(71) Applicant: Allakos Inc., Redwood City, CA (US)

(72) Inventors: Bradford Andrew YOUNGBLOOD, Burlingame, CA (US); Bhupinder SINGH, Paradise Valley, AZ (US); Amol KAMBOJ, Redwood City, CA (US); Simon GREENWOOD, Redwood City, CA (US); Henrik RASMUSSEN, Annapolis, MD (US)

(21) Appl. No.: 17/430,256

(22) PCT Filed: Feb. 14, 2020
(86) PCT No.: PCT/US2020/018405

§ 371 Date: Aug. 11, 2021

Related U.S. Application Data
(60) Provisional application No. 62/925,704, filed on Oct. 24, 2019.
(60) Provisional application No. 62/806,604, filed on Feb. 15, 2019.

(51) Int. Cl. C07K 16/28; G01N 33/50; A61K 39/395; A61K 45/06; A61P 1/04

(52) U.S. Cl. CPC C07K 16/2803; G01N 33/5091; A61K 39/3955; A61K 2039/505; A61P 1/04; C07K 2317/732; A61K 45/06

(57) ABSTRACT

The present disclosure provides methods for the treatment of mast cell gastritis, mast cell esophagitis, mast cell colitis, mast cell enteritis, mast cell duodenitis, and/or mast cell gastroenteritis. In particular, the present disclosure provides methods for the treatment of mast cell gastritis, mast cell esophagitis, mast cell colitis, mast cell enteritis, mast cell duodenitis, and/or mast cell gastroenteritis through administration of antibodies that bind to human Siglec-8 or compositions comprising said antibodies. The present disclosure also provides articles of manufacture or kits comprising antibodies that bind to human Siglec-8 for the treatment of mast cell gastritis, mast cell esophagitis, mast cell colitis, mast cell enteritis, mast cell duodenitis, and/or mast cell gastroenteritis.

### CROSS-REFERENCE TO RELATED APPLICATIONS

This application claims priority to U.S. Provisional Application Ser. No. 62/806,604, filed Feb. 15, 2019, and 62/925,704, filed Oct. 24, 2019, the disclosures of each of which are incorporated herein by reference in their entirety.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 701712001040SEQLIST.TXT, date recorded: Feb. 13, 2020, size: 106 KB).

### FIELD OF THE INVENTION

The present disclosure relates to methods for treating mast cell gastritis, mast cell esophagitis, mast cell colitis, mast cell enteritis, mast cell duodenitis, and/or mast cell gastroenteritis by administration of antibodies that bind to human Siglec-8 and compositions comprising said antibodies.

### BACKGROUND

Siglec-8, a member of the CD33-related family of sialic acid-binding, immunoglobulin-like lectins (Siglecs), is a transmembrane cell surface protein with restricted tissue distribution, expressed selectively on the surface of eosinophils, mast cells and, at lower levels, on basophils. Siglec-8 contains 3 extracellular immunoglobulin-like domains, a transmembrane region, and a cytoplasmic tail containing 2 tyrosine-based signaling motifs including an immunoreceptor tyrosine-based inhibitory motif with inhibitory function. Engagement of Siglec-8 in mast cells can result in inhibition of mediator release, and in eosinophils can induce apoptosis (Bochner, B. (2009) Clin. Exp. Allergy 39:317-324).

There are no FDA-approved treatments for gastritis and/or gastroenteritis with increased mast cells. Current therapies and disease management for these patients include a plethora of various approaches, including proton pump inhibitors, antihistamines, restricted/elemental diets, mast cell stabilizers, systemic or oral corticosteroids, and occasional off-label use of immunomodulatory biologics. As such, there remains a need for an effective treatment for these and related disorders.

All references cited herein, including patent applications, patent publications, and scientific literature, are herein incorporated by reference in their entirety, as if each individual reference were specifically and individually indicated to be incorporated by reference.

### BRIEF SUMMARY

To meet this and other needs, the present disclosure relates, inter alia, to methods of treating or preventing mast cell gastritis, mast cell esophagitis, mast cell enteritis, mast cell colitis, mast cell duodenitis, and/or mast cell gastroenteritis by administration of antibodies that bind to human Siglec-8 and/or compositions comprising said antibodies. These methods allow for treatment of individuals with one or more symptoms of esophagitis, gastritis, enteritis, duodenitis, and/or gastroenteritis that have elevated tissue mast cells (e.g., and do not meet clinical criteria for eosinophilic esophagitis, gastritis, colitis, duodenitis, enteritis, and/or gastroenteritis).

Accordingly, certain aspects of the present disclosure relate to methods for treating or preventing one or more symptoms of gastritis, enteritis, duodenitis, or gastroenteritis in an individual, comprising: (a) detecting number of mast cells from a first sample obtained from gastric, duodenal, jejunal, ileal, or colonic mucosa of the individual; (b) detecting number of eosinophils from a second sample obtained from gastric, duodenal, jejunal, ileal, or colonic mucosa of the individual; and (c) if the first sample has an increased number of mast cells as compared to a mast cell reference and the second sample does not have increased number of eosinophils as compared to an eosinophil reference, administering to the individual an effective amount of a composition comprising an antibody that binds to human Siglec-8. Other aspects of the present disclosure relate to methods for treating or preventing mast cell gastritis, mast cell enteritis, mast cell duodenitis, or mast cell gastroenteritis in an individual, comprising: (a) detecting number of mast cells from a first sample obtained from gastric, duodenal, jejunal, ileal, or colonic mucosa of the individual; (b) detecting number of eosinophils from a second sample obtained from gastric, duodenal, jejunal, ileal, or colonic mucosa of the individual; and (c) if the first sample has an increased number of mast cells as compared to a mast cell reference and the second sample does not have increased number of eosinophils as compared to an eosinophil reference, administering to the individual an effective amount of a composition comprising an antibody that binds to human Siglec-8. Other aspects of the present disclosure relate to methods for treating an individual having one or more symptoms of gastritis, enteritis, duodenitis, or gastroenteritis, comprising: (a) detecting number of mast cells from a first sample obtained from gastric, duodenal, jejunal, ileal, or colonic mucosa of the individual; (b) detecting number of eosinophils from a second sample obtained from gastric, duodenal, jejunal, ileal, or colonic mucosa of the individual; and (c) if the first sample has an increased number of mast cells as compared to a mast cell reference and the second sample does not have increased number of eosinophils as compared to an eosinophil reference, administering to the individual an effective amount of a composition comprising an antibody that binds to human Siglec-8.

Other aspects of the present disclosure relate to methods for treating or preventing one or more symptoms of gastritis, enteritis, duodenitis, or gastroenteritis in an individual comprising administering to the individual an effective amount of a composition comprising an antibody that binds to human Siglec-8, wherein the individual has an increased number of mast cells in at least a portion of the gastric, duodenal, jejunal, ileal, or colonic mucosa as compared to a mast cell reference, and wherein the individual does not have increased number of eosinophils in at least a portion of the gastric, duodenal, jejunal, ileal, or colonic mucosa as compared to an eosinophil reference. Other aspects of the present disclosure relate to methods for treating or preventing mast cell gastritis, mast cell enteritis, mast cell duodenitis, or mast cell gastroenteritis in an individual comprising administering to the individual an effective amount of a composition comprising an antibody that binds to human Siglec-8, wherein the individual has an increased number of mast cells in at least a portion of the gastric, duodenal, jejunal, ileal, or colonic mucosa as compared to a mast cell reference, and wherein the individual does not have increased number of eosinophils in at least a portion of the gastric, duodenal, jejunal, ileal, or colonic mucosa as compared to an eosinophil reference. Other aspects of the present disclosure relate to methods for treating an individual having one or more symptoms of gastritis, enteritis, duodenitis, or gastroenteritis comprising administering to the individual an effective amount of a composition comprising an antibody that binds to human Siglec-8, wherein the individual has an increased number of mast cells in at least a portion of the gastric, duodenal, jejunal, ileal, or colonic mucosa as compared to a mast cell reference, and wherein the individual does not have increased number of eosinophils in at least a portion of the gastric, duodenal, jejunal, ileal, or colonic mucosa as compared to an eosinophil reference. In some embodiments, a first sample obtained from the gastric, duodenal, jejunal, ileal, or colonic mucosa of the individual has an increased number of mast cells as compared to the mast cell reference. In some embodiments, a second sample obtained from the gastric, duodenal, jejunal, ileal, or colonic mucosa of the individual does not have increased number of eosinophils as compared to the eosinophil reference.

In some embodiments that may be combined with any other embodiments described herein, the first and the second samples are the same. In some embodiments, the first and the second samples are from the same type of tissue. In some embodiments, one or both of the first and second samples is/are from a gastric or duodenal biopsy. In some embodiments, one or both of the first and second samples is/are from an esophago-gastro-duodenoscopy (EGD) with biopsy. In some embodiments, the first sample has at least three high-power fields (HPFs) that each have a mast cell count of 30 or more mast cells per HPF. In some embodiments, the first sample has at least three high-power fields (HPFs) that each have a mast cell count of 25 or more mast cells per HPF. In some embodiments, the first sample has at least three high-power fields (HPFs) that each have a mast cell count of 20 or more mast cells per HPF. In some embodiments, the first sample has at least two high-power fields (HPFs) that each have a mast cell count of 30 or more mast cells per HPF. In some embodiments, the first sample has at least two high-power fields (HPFs) that each have a mast cell count of 25 or more mast cells per HPF. In some embodiments, the first sample has at least two high-power fields (HPFs) that each have a mast cell count of 20 or more mast cells per HPF. In some embodiments, the first sample has at least one high-power field (HPF) that has a mast cell count of 30 or more mast cells. In some embodiments, the first sample has at least one high-power field (HPF) that has a mast cell count of 25 or more mast cells. In some embodiments, the first sample has at least one high-power field (HPF) that has a mast cell count of 20 or more mast cells. In some embodiments, mast cells are detected by immunohistochemical (IHC) staining for tryptase. CD117 (c-kit), or IgE receptor. In some embodiments, the second sample has one or more HPFs that each have an eosinophil count of less than 30 eosinophils per HPF. In some embodiments, the second sample is obtained from the gastric mucosa of the individual, and the second sample does not have at least five HPFs that each have an eosinophil count of 30 or more eosinophils per HPF. In some embodiments, the second sample is obtained from the duodenal mucosa of the individual, and the second sample does not have at least three HPFs that each have an eosinophil count of 30 or more eosinophils per HPF. In some embodiments, the number of mast cells in the first sample is detected 45 days or less prior to administration of the composition.

In some embodiments according to any of the embodiments described herein, the individual has, or has been diagnosed with, gastroesophageal reflux disease (GERD) (e.g., prior to treatment with an anti-Siglec-8 antibody). In some embodiments, the individual is refractory to antacids, H2 blockers, and/or proton pump inhibitors. In some embodiments, the individual has, or has been diagnosed with, irritable bowel syndrome (IBS) (e.g., prior to treatment with an anti-Siglec-8 antibody). In some embodiments, the individual has, or has been diagnosed with, functional dyspepsia (e.g., prior to treatment with an anti-Siglec-8 antibody). In some embodiments, the individual has, or has been diagnosed with, one or more of: abdominal pain, abdominal cramping, nausea, vomiting, diarrhea, bloating, and early satiety without identifiable cause (e.g., prior to treatment with an anti-Siglec-8 antibody). In some embodiments, the individual is refractory and/or unresponsive to pharmacologic and/or dietary intervention. In some embodiments, the individual previously had (e.g., prior to treatment with an anti-Siglec-8 antibody), or was diagnosed with (e.g., has a previous history of), eosinophilic gastritis but is currently symptomatic without elevated eosinophils. In some embodiments, the individual has had (e.g., prior to treatment with an anti-Siglec-8 antibody), or has previously been diagnosed with (e.g., prior to treatment with an anti-Siglec-8 antibody), eosinophilic gastritis, and the individual has one or more symptoms of eosinophilic gastritis without elevated eosinophils. In some embodiments, the individual previously had (e.g., prior to treatment with an anti-Siglec-8 antibody), or was diagnosed with (e.g., has a previous history of), eosinophilic gastroenteritis but is currently symptomatic without elevated eosinophils. In some embodiments, the individual has had (e.g., prior to treatment with an anti-Siglec-8 antibody), or has previously been diagnosed with (e.g., prior to treatment with an anti-Siglec-8 antibody), eosinophilic gastroenteritis, and the individual has one or more symptoms of eosinophilic gastroenteritis without elevated eosinophils. In some embodiments, the individual has had (e.g., prior to treatment with an anti-Siglec-8 antibody), or has previously been diagnosed with (e.g., prior to treatment with an anti-Siglec-8 antibody), eosinophilic enteritis, and the individual has one or more symptoms of eosinophilic enteritis without elevated eosinophils. In some embodiments, the individual has had (e.g., prior to treatment with an anti-Siglec-8 antibody), or has previously been diagnosed with (e.g., prior to treatment with an anti-Siglec-8 antibody), eosinophilic duodenitis, and the individual has one or more symptoms of eosinophilic duodenitis without elevated eosinophils. In some embodiments, the individual has, or has been diagnosed with, functional dyspepsia (e.g., prior to treatment with an anti-Siglec-8 antibody). In some embodiments, one or both of a number or activity of mast cells in a sample obtained from the gastric, duodenal, jejunal, ileal, or colonic mucosa of the individual are reduced after administration of the composition as compared to a baseline level before administration of the composition. In some embodiments, prior to administration of the composition, the individual has failed or is not adequately controlled by one or more standard-of-care treatments for gastritis or gastroenteritis. In some embodiments, the one or more standard-of-care treatments for gastritis or gastroenteritis are selected from the group consisting of proton pump inhibitor (PPI) treatment, corticosteroid treatment, and dietary treatment. In some embodiments, one or more symptom(s) of gastritis or gastroenteritis in the individual are reduced after administration of the composition as compared to a baseline level before administration of the composition. In some embodiments, one or more symptom(s) of gastritis, duodenitis, enteritis, or gastroenteritis in the individual are reduced by at least 50%, at least 55%, at least 60%, or at least 65% after administration of the composition as compared to a baseline level before administration of the composition. In some embodiments, one or more of abdominal pain, nausea, vomiting, loss of appetite, abdominal cramping, fullness before finishing a meal, bloating, diarrhea, and liquid or watery stools in the individual are reduced after administration of the composition as compared to a baseline level before administration of the composition.

Other aspects of the present disclosure relate to methods for treating or preventing one or more symptoms of esophagitis in an individual, comprising: (a) detecting number of mast cells from a first sample obtained from esophageal mucosa of the individual; (b) detecting number of eosinophils from a second sample obtained from the esophageal mucosa of the individual; and (c) if the first sample has an increased number of mast cells as compared to a mast cell reference and the second sample does not have increased number of eosinophils as compared to an eosinophil reference, administering to the individual an effective amount of a composition comprising an antibody that binds to human Siglec-8. Other aspects of the present disclosure relate to methods for treating or preventing mast cell esophagitis in an individual, comprising: (a) detecting number of mast cells from a first sample obtained from esophageal mucosa of the individual; (b) detecting number of eosinophils from a second sample obtained from the esophageal mucosa of the individual; and (c) if the first sample has an increased number of mast cells as compared to a mast cell reference and the second sample does not have increased number of eosinophils as compared to an eosinophil reference, administering to the individual an effective amount of a composition comprising an antibody that binds to human Siglec-8. Other aspects of the present disclosure relate to methods for treating an individual having one or more symptoms of esophagitis, comprising: (a) detecting number of mast cells from a first sample obtained from esophageal mucosa of the individual; (b) detecting number of eosinophils from a second sample obtained from the esophageal mucosa of the individual; and (c) if the first sample has an increased number of mast cells as compared to a mast cell reference and the second sample does not have increased number of eosinophils as compared to an eosinophil reference, administering to the individual an effective amount of a composition comprising an antibody that binds to human Siglec-8.

Other aspects of the present disclosure relate to methods for treating or preventing one or more symptoms of esophagitis in an individual comprising administering to the individual an effective amount of a composition comprising an antibody that binds to human Siglec-8, wherein the individual has an increased number of mast cells in at least a portion of the esophageal mucosa as compared to a mast cell reference, and wherein the individual does not have increased number of eosinophils in at least a portion of the esophageal mucosa as compared to an eosinophil reference. Other aspects of the present disclosure relate to methods for treating or preventing mast cell esophagitis in an individual comprising administering to the individual an effective amount of a composition comprising an antibody that binds to human Siglec-8, wherein the individual has an increased number of mast cells in at least a portion of the esophageal mucosa as compared to a mast cell reference, and wherein the individual does not have increased number of eosinophils in at least a portion of the esophageal mucosa as compared to an eosinophil reference. Other aspects of the present disclosure relate to methods for treating an individual having one or more symptoms of esophagitis comprising administering to the individual an effective amount of a composition comprising an antibody that binds to human Siglec-8, wherein the individual has an increased number of mast cells in at least a portion of the esophageal mucosa as compared to a mast cell reference, and wherein the individual does not have increased number of eosinophils in at least a portion of the esophageal mucosa as compared to an eosinophil reference. In some embodiments, a first sample obtained from the esophageal mucosa of the individual has an increased number of mast cells as compared to the mast cell reference. In some embodiments, a second sample obtained from the esophageal mucosa of the individual does not have increased number of eosinophils as compared to the eosinophil reference.

In some embodiments, the first and the second samples are the same. In some embodiments, one or both of the first and second samples is/are from an esophageal biopsy sample. In some embodiments, one or both of the first and second samples is/are from an esophago-gastro-duodenoscopy (EGD) with biopsy. In some embodiments, the first sample has at least one high-power field (HPF) with a mast cell count of 10 or more mast cells per HPF. In some embodiments, the first sample has at least one high-power field (HPF) with a mast cell count of 15 or more mast cells per HPF. In some embodiments, mast cells are detected by immunohistochemical (IHC) staining for tryptase, CD117 (c-kit), or IgE receptor. In some embodiments, the second sample has one or more HPFs with an eosinophil count of less than 10 eosinophils per HPF. In some embodiments, the second sample does not have an HPF with an eosinophil count of 10 or more eosinophils per HPF. In some embodiments, the second sample has one or more HPFs with an eosinophil count of less than 15 eosinophils per HPF. In some embodiments, the second sample does not have an HPF with an eosinophil count of 15 or more eosinophils per HPF.

In some embodiments, one or more of a number, activity, or location of mast cells in a sample obtained from the esophageal mucosa of the individual is reduced after administration of the composition as compared to a baseline level before administration of the composition. In some embodiments, prior to administration of the composition, the individual has failed or is not adequately controlled by one or more standard-of-care treatments for esophagitis. In some embodiments, the one or more symptom(s) of esophagitis in the individual are reduced after administration of the composition as compared to a baseline level before administration of the composition. In some embodiments, the one or more symptom(s) of esophagitis in the individual are reduced by at least 50%, at least 55%, at least 60%, or at least 65% after administration of the composition as compared to a baseline level before administration of the composition. In some embodiments, one or more of heartburn, nausea, dysphagia/difficulty swallowing, vomiting, abdominal pain, cough, food impaction, early satiety, loss of appetite, chest pain, feeding intolerance or refusal, and gastroesophageal reflux in the individual are reduced after administration of the composition as compared to a baseline level before administration of the composition.

Other aspects of the present disclosure relate to methods for treating or preventing one or more symptoms of colitis (e.g., ulcerative colitis) in an individual, comprising: (a) detecting number of mast cells from a first sample obtained from colonic mucosa of the individual; (b) detecting number of eosinophils from a second sample obtained from the colonic mucosa of the individual; and (c) if the first sample has an increased number of mast cells as compared to a mast cell reference and the second sample does not have increased number of eosinophils as compared to an eosinophil reference, administering to the individual an effective amount of a composition comprising an antibody that binds to human Siglec-8. Other aspects of the present disclosure relate to methods for treating or preventing mast cell colitis (e.g., ulcerative colitis) in an individual, comprising: (a) detecting number of mast cells from a first sample obtained from colonic mucosa of the individual; (b) detecting number of eosinophils from a second sample obtained from the colonic mucosa of the individual; and (c) if the first sample has an increased number of mast cells as compared to a mast cell reference and the second sample does not have increased number of eosinophils as compared to an eosinophil reference, administering to the individual an effective amount of a composition comprising an antibody that binds to human Siglec-8. Other aspects of the present disclosure relate to methods for treating an individual having one or more symptoms of colitis (e.g., ulcerative colitis), comprising: (a) detecting number of mast cells from a first sample obtained from colonic mucosa of the individual: (b) detecting number of eosinophils from a second sample obtained from the colonic mucosa of the individual; and (c) if the first sample has an increased number of mast cells as compared to a mast cell reference and the second sample does not have increased number of eosinophils as compared to an eosinophil reference, administering to the individual an effective amount of a composition comprising an antibody that binds to human Siglec-8.

Other aspects of the present disclosure relate to methods for treating or preventing one or more symptoms of colitis (e.g., ulcerative colitis) in an individual comprising administering to the individual an effective amount of a composition comprising an antibody that binds to human Siglec-8, wherein the individual has an increased number of mast cells in at least a portion of the colonic mucosa as compared to a mast cell reference, and wherein the individual does not have increased number of eosinophils in at least a portion of the colonic mucosa as compared to an eosinophil reference. Other aspects of the present disclosure relate to methods for treating or preventing mast cell colitis (e.g., ulcerative colitis) in an individual comprising administering to the individual an effective amount of a composition comprising an antibody that binds to human Siglec-8, wherein the individual has an increased number of mast cells in at least a portion of the colonic mucosa as compared to a mast cell reference, and wherein the individual does not have increased number of eosinophils in at least a portion of the colonic mucosa as compared to an eosinophil reference. Other aspects of the present disclosure relate to methods for treating an individual having one or more symptoms of colitis (e.g., ulcerative colitis) comprising administering to the individual an effective amount of a composition comprising an antibody that binds to human Siglec-8, wherein the individual has an increased number of mast cells in at least a portion of the colonic mucosa as compared to a mast cell reference, and wherein the individual does not have increased number of eosinophils in at least a portion of the colonic mucosa as compared to an eosinophil reference. In some embodiments, a first sample obtained from the colonic mucosa of the individual has an increased number of mast cells as compared to the mast cell reference. In some embodiments, a second sample obtained from the colonic mucosa of the individual does not have increased number of eosinophils as compared to the eosinophil reference.

In some embodiments, the first sample has at least one high-power field (HPF) with a mast cell count of 20 or more, 25 or more, 30 or more, or 20-30 mast cells per HPF. In some embodiments, the second sample has one or more HPFs with an eosinophil count of less than 60 eosinophils per HPF. In some embodiments, the second sample does not have one or more HPFs with an eosinophil count of greater than 60 eosinophils per HPF. In some embodiments, the first and the second samples are the same. In some embodiments, mast cells are detected by immunohistochemical (IHC) staining for tryptase, CD117, or IgE receptor. In some embodiments, the number of mast cells in the first sample is detected 45 days or less prior to administration of the composition. In some embodiments, the individual has had, or has previously been diagnosed with, eosinophilic colitis, and the individual has one or more symptoms of eosinophilic colitis without elevated eosinophils. In some embodiments, one or both of a number or activity of mast cells in a sample obtained from the colonic mucosa of the individual are reduced after administration of the composition as compared to a baseline level before administration of the composition. In some embodiments, prior to administration of the composition, the individual has failed or is not adequately controlled by one or more standard-of-care treatments for colitis. In some embodiments, the one or more symptom(s) of colitis in the individual are reduced after administration of the composition as compared to a baseline level before administration of the composition. In some embodiments, the one or more symptom(s) of colitis in the individual are reduced by at least 50%, at least 55%, at least 60%, or at least 65% after administration of the composition as compared to a baseline level before administration of the composition.

In some embodiments that may be combined with any other embodiments described herein, the composition is administered by intravenous infusion. In some embodiments, the composition is administered by intravenous infusion once a month for 3 or more months, every 4 weeks, or every 28 days. In some embodiments, the composition is administered by intravenous infusion once per cycle for 1, 2, 3, 4, 5, or 6 cycles, wherein each cycle is 1 month, 4 weeks, or 28 days. In some embodiments, the composition is administered by subcutaneous injection. In some embodiments, the composition is administered by intravenous infusion at one or more doses comprising between about 0.3 mg/kg and about 3.0 mg/kg of the antibody. In some embodiments, the method comprises administering to the individual a first dose comprising about 0.3 mg/kg of the antibody, a second dose comprising about 1.0 mg/kg of the antibody, and a third dose comprising about 3.0 mg/kg of the antibody. In some embodiments, the method comprises administering to the individual a first dose comprising about 0.3 mg/kg of the antibody on Day 1, a second dose comprising about 1.0 mg/kg of the antibody between Day 26 and Day 32, a third dose comprising about 3.0 mg/kg of the antibody between Day 54 and Day 60, a fourth dose comprising about 3.0 mg/kg of the antibody between Day 82 and Day 88, a fifth dose comprising about 3.0 mg/kg of the antibody between Day 110 and Day 116, and a sixth dose comprising about 3.0 mg/kg of the antibody between Day 138 and Day 144.

In some embodiments that may be combined with any other embodiments described herein, the antibody comprises a Fc region and N-glycoside-linked carbohydrate chains linked to the Fc region, wherein less than 50% of the N-glycoside-linked carbohydrate chains of the antibody in the composition contain a fucose residue. In some embodiments, substantially none of the N-glycoside-linked carbohydrate chains of the antibody in the composition contain a fucose residue. In some embodiments, the antibody comprises a heavy chain variable region and a light chain variable region, wherein the heavy chain variable region comprises (i) HVR-H1 comprising the amino acid sequence of SEQ ID NO:61, (ii) HVR-H2 comprising the amino acid sequence of SEQ ID NO:62, and (iii) HVR-H3 comprising the amino acid sequence of SEQ ID NO:63; and/or wherein the light chain variable region comprises (i) HVR-L1 comprising the amino acid sequence of SEQ ID NO:64, (ii) HVR-L2 comprising the amino acid sequence of SEQ ID NO:65, and (iii) HVR-L3 comprising the amino acid sequence of SEQ ID NO:66. In some embodiments, the antibody comprises a heavy chain variable region and a light chain variable region, wherein the heavy chain variable region comprises (i) HVR-H1 comprising the amino acid sequence of SEQ ID NO:61, (ii) HVR-H2 comprising the amino acid sequence of SEQ ID NO:62, and (iii) HVR-H3 comprising the amino acid sequence selected from SEQ ID NOs:67-70; and/or wherein the light chain variable region comprises (i) HVR-L1 comprising the amino acid sequence of SEQ ID NO:64, (ii) HVR-L2 comprising the amino acid sequence of SEQ ID NO:65, and (iii) HVR-L3 comprising the amino acid sequence of SEQ ID NO:71. In some embodiments, the antibody comprises a heavy chain variable region comprising the amino acid sequence of SEQ ID NO:6; and/or a light chain variable region comprising the amino acid sequence selected from SEQ ID NO:16 or 21. In some embodiments, the antibody comprises a heavy chain variable region comprising the amino acid sequence selected from SEQ ID NOs: 11-14; and/or a light chain variable region comprising the amino acid sequence selected from SEQ ID NOs:23-24. In some embodiments, the antibody comprises a heavy chain variable region comprising the amino acid sequence selected from SEQ ID NOs:2-14; and/or a light chain variable region comprising the amino acid sequence selected from SEQ ID NOs:16-24. In some embodiments, the antibody comprises a heavy chain variable region comprising the amino acid sequence selected from SEQ ID NOs:2-10; and/or a light chain variable region comprising the amino acid sequence selected from SEQ ID NOs:16-22. In some embodiments, the antibody comprises: (a) heavy chain variable region comprising: (1) an HC-FR1 comprising the amino acid sequence selected from SEQ ID NOs:26-29; (2) an HVR-H1 comprising the amino acid sequence of SEQ ID NO:61; (3) an HC-FR2 comprising the amino acid sequence selected from SEQ ID NOs:31-36; (4) an HVR-H2 comprising the amino acid sequence of SEQ ID NO:62; (5) an HC-FR3 comprising the amino acid sequence selected from SEQ ID NOs:38-43; (6) an HVR-H3 comprising the amino acid sequence of SEQ ID NO:63; and (7) an HC-FR4 comprising the amino acid sequence selected from SEQ ID NOs:45-46, and/or (b) a light chain variable region comprising: (1) an LC-FR1 comprising the amino acid sequence selected from SEQ ID NOs:48-49; (2) an HVR-L1 comprising the amino acid sequence of SEQ ID NO:64; (3) an LC-FR2 comprising the amino acid sequence selected from SEQ ID NOs:51-53; (4) an HVR-L2 comprising the amino acid sequence of SEQ ID NO:65; (5) an LC-FR3 comprising the amino acid sequence selected from SEQ ID NOs:55-58; (6) an HVR-L3 comprising the amino acid sequence of SEQ ID NO:66; and (7) an LC-FR4 comprising the amino acid sequence of SEQ ID NO:60. In some embodiments, the antibody comprises: (a) heavy chain variable region comprising: (1) an HC-FR1 comprising the amino acid sequence of SEQ ID NO:26; (2) an HVR-H1 comprising the amino acid sequence of SEQ ID NO:61; (3) an HC-FR2 comprising the amino acid sequence of SEQ ID NO:34; (4) an HVR-H2 comprising the amino acid sequence of SEQ ID NO:62; (5) an HC-FR3 comprising the amino acid sequence of SEQ ID NO:38; (6) an HVR-H3 comprising the amino acid sequence of SEQ ID NO:63; and (7) an HC-FR4 comprising the amino acid sequence of SEQ ID NOs:45; and/or (b) a light chain variable region comprising: (1) an LC-FR1 comprising the amino acid sequence of SEQ ID NO:48; (2) an HVR-L1 comprising the amino acid sequence of SEQ ID NO:64; (3) an LC-FR2 comprising the amino acid sequence of SEQ ID NO:51; (4) an HVR-L2 comprising the amino acid sequence of SEQ ID NO:65; (5) an LC-FR3 comprising the amino acid sequence of SEQ ID NO:55; (6) an HVR-L3 comprising the amino acid sequence of SEQ ID NO:66; and (7) an LC-FR4 comprising the amino acid sequence of SEQ ID NO:60. In some embodiments, the antibody comprises: (a) heavy chain variable region comprising: (1) an HC-FR1 comprising the amino acid sequence of SEQ ID NO:26; (2) an HVR-H1 comprising the amino acid sequence of SEQ ID NO:61; (3) an HC-FR2 comprising the amino acid sequence of SEQ ID NO:34; (4) an HVR-H2 comprising the amino acid sequence of SEQ ID NO:62; (5) an HC-FR3 comprising the amino acid sequence of SEQ ID NO:38; (6) an HVR-H3 comprising the amino acid sequence of SEQ ID NO:63; and (7) an HC-FR4 comprising the amino acid sequence of SEQ ID NOs:45; and/or (b) a light chain variable region comprising: (1) an LC-FR1 comprising the amino acid sequence of SEQ ID NO:48; (2) an HVR-L1 comprising the amino acid sequence of SEQ ID NO:64; (3) an LC-FR2 comprising the amino acid sequence of SEQ ID NO:51; (4) an HVR-L2 comprising the amino acid sequence of SEQ ID NO:65; (5) an LC-FR3 comprising the amino acid sequence of SEQ ID NO:58; (6) an HVR-L3 comprising the amino acid sequence of SEQ ID NO:66; and (7) an LC-FR4 comprising the amino acid sequence of SEQ ID NO:60. In some embodiments, the antibody comprises: a heavy chain variable region comprising (i) HVR-H1 comprising the amino acid sequence of SEQ ID NO:88, (ii) HVR-H2 comprising the amino acid sequence of SEQ ID NO:91, and (iii) HVR-H3 comprising the amino acid sequence of SEQ ID NO:94; and/or a light chain variable region comprising (i) HVR-L1 comprising the amino acid sequence of SEQ ID NO:97, (ii) HVR-L2 comprising the amino acid sequence of SEQ ID NO:100, and (iii) HVR-L3 comprising the amino acid sequence of SEQ ID NO:103; a heavy chain variable region comprising (i) HVR-H1 comprising the amino acid sequence of SEQ ID NO:89, (ii) HVR-H2 comprising the amino acid sequence of SEQ ID NO:92, and (iii) HVR-H3 comprising the amino acid sequence of SEQ ID NO:95; and/or a light chain variable region comprising (i) HVR-L1 comprising the amino acid sequence of SEQ ID NO:98, (ii) HVR-L2 comprising the amino acid sequence of SEQ ID NO:101, and (iii) HVR-L3 comprising the amino acid sequence of SEQ ID NO:104; or a heavy chain variable region comprising (i) HVR-H1 comprising the amino acid sequence of SEQ ID NO:90, (ii) HVR-H2 comprising the amino acid sequence of SEQ ID NO:93, and (iii) HVR-H3 comprising the amino acid sequence of SEQ ID NO:96; and/or a light chain variable region comprising (i) HVR-L1 comprising the amino acid sequence of SEQ ID NO:99, (ii) HVR-L2 comprising the amino acid sequence of SEQ ID NO:102, and (iii) HVR-L3 comprising the amino acid sequence of SEQ ID NO:105. In some embodiments, the antibody comprises: a heavy chain variable region comprising the amino acid sequence of SEQ ID NO:106; and/or a light chain variable region comprising the amino acid sequence of SEQ ID NO:109; a heavy chain variable region comprising the amino acid sequence of SEQ ID NO:107; and/or a light chain variable region comprising the amino acid sequence of SEQ ID NO:110; or a heavy chain variable region comprising the amino acid sequence of SEQ ID NO:108; and/or a light chain variable region comprising the amino acid sequence of SEQ ID NO:111. In some embodiments, the antibody binds to a human Siglec-8 and a non-human primate Siglec-8. In some embodiments, the non-human primate is a baboon. In some embodiments, the antibody binds to an epitope in Domain 1 of human Siglec-8, wherein Domain 1 comprises the amino acid sequence of SEQ ID NO:112. In some embodiments, the antibody binds to an epitope in Domain 3 of human Siglec-8, wherein Domain 3 comprises the amino acid sequence of SEQ ID NO:114. In some embodiments, the antibody binds to the same epitope as antibody 4F11. In some embodiments, the antibody binds to an epitope in Domain 2 or Domain 3 of human Siglec-8. In some embodiments, Domain 2 comprises the amino acid sequence of SEQ ID NO:113. In some embodiments, the antibody binds to the same epitope as antibody 1C3. In some embodiments, Domain 3 comprises the amino acid sequence of SEQ ID NO:114. In some embodiments, the antibody binds to the same epitope as antibody 1H10. In some embodiments, the antibody binds to an epitope in Domain 1 of human Siglec-8 and competes with antibody 4F11 for binding to Siglec-8. In some embodiments, the antibody does not compete with antibody 2E2 for binding to Siglec-8. In some embodiments, the antibody is not antibody 2E2. In some embodiments, Domain 1 comprises the amino acid sequence of SEQ ID NO:112. In some embodiments, the antibody is a human antibody, a humanized antibody, or a chimeric antibody. In some embodiments, the antibody comprises a heavy chain Fc region comprising a human IgG Fc region. In some embodiments, the human IgG Fc region comprises a human IgG1 Fc region. In some embodiments, the human IgG1 Fc region is non-fucosylated. In some embodiments, the human IgG Fc region comprises a human IgG4 Fc region. In some embodiments, the human IgG4 Fc region comprises the amino acid substitution S228P, wherein the amino acid residues are numbered according to the EU index as in Kabat. In some embodiments, the antibody depletes blood eosinophils and/or inhibits mast cell activation. In some embodiments, the antibody has been engineered to improve antibody-dependent cell-mediated cytotoxicity (ADCC) activity. In some embodiments, the antibody comprises at least one amino acid substitution in the Fc region that improves ADCC activity. In some embodiments, at least one or two of the heavy chains of the antibody is non-fucosylated. In some embodiments, the antibody comprises a heavy chain comprising the amino acid sequence of SEQ ID NO:75; and/or a light chain comprising the amino acid sequence selected from SEQ ID NO:76 or 77. In some embodiments, the antibody is a monoclonal antibody.

In some embodiments that may be combined with any other embodiments described herein, the composition is administered in combination with one or more additional therapeutic agent(s) for treating or preventing gastritis, gastroenteritis, or esophagitis. In some embodiments, the one or more additional therapeutic agent(s) for treating or preventing gastritis, gastroenteritis, or esophagitis are selected from the group consisting of PPIs, systemic corticosteroids, topical corticosteroids, antihistamines, mast cell stabilizers. H-2 blockers, anti-IgE antibodies, calcineurin inhibitors, immunomodulatory agents, and immunosuppressive agents. In some embodiments, the individual is a human. In some embodiments, the composition is a pharmaceutical composition comprising the antibody and a pharmaceutically acceptable carrier.

Other aspects of the present disclosure relate to articles of manufacture or kits comprising a medicament comprising a composition comprising an antibody that binds to human Siglec-8 and a package insert comprising instructions for administration of the medicament in an individual in need thereof according to any one of the above embodiments. Other aspects of the present disclosure relate to articles of manufacture or kits comprising a medicament comprising an antibody that binds to human Siglec-8 and a package insert comprising instructions for administration of the medicament in an individual in need thereof according to any one of the above embodiments.

It is to be understood that one, some, or all of the properties of the various embodiments described herein may be combined to form other embodiments of the present disclosure. These and other aspects of the present disclosure will become apparent to one of skill in the art. These and other embodiments of the present disclosure are further described by the detailed description that follows.

### BRIEF DESCRIPTION OF THE DRAWINGS

FIG. 1 provides a schematic diagram illustrating the pathogenesis of eosinophilic gastrointestinal diseases (EGIDs).

FIGS. 2A & 2B show the distribution (FIG. 2A) and baseline characteristics (FIG. 2B) of symptomatic patients with suspected EG/EEn who did not meet histopathologic entry criteria for mucosal eosinophilia for a Phase 2 study of anti-Siglec-8 antibody in patients with EG/EEn. In FIG. 2B, ^{a}=medical history at screening of asthma, rhinitis, food allergy, atopic dermatitis, seasonal allergy, environmental allergy, or pollen allergy.

FIGS. 3A & 3B show that mast cells are consistently elevated in stomach (FIG. 3A) and duodenal (FIG. 3B) biopsies in symptomatic patients. Horizontal shading indicates normal mast cell levels (see. e.g., Hahn et al. (2007) Am. J. Surg. Pathol. 31:1669-1676; Tison et al. (2010) J. Allergy Clin. Immunol. 125:AB182; Walker et 71. (2009) Aliment. Pharmacol. Ther. 29:765-773; Martinez et al. (2013) Gut 62:1160-1168; and Doyle et al. (2014) Am. J. Surg. Pathol. 38:832-843).

FIG. 4 shows the mean symptom intensity (0-10) during screening for each of 8 symptoms for patients with ≥30 eosinophils/hpf (n=71; light) or patients with <30 eosinophils/hpf but ≥30 mast cells/hpf (n=16: dark). For the cohort with ≥30 eosinophils/hpf, one patient was missing symptom data and did not enroll.

FIGS. 5A & 5B show two individual patient case studies.

FIGS. 6A & 6B show that increased activation of mast cells is seen in tissues where only mast cells (and not eosinophils) are elevated. In FIG. 6A, human gastric biopsy tissue was processed into single cells, followed by quantification of mast cells (CD117+ Siglec-8+) and eosinophils (CD117− Siglec-8+) by flow cytometry. In FIG. 6B, mast cells identified in FIG. 6A were further analyzed for the activation and degranulation marker CD63 using flow cytometry. Staining with an antibody specific for CD63 or control antibody are shown.

FIG. 7 shows the mean and median change in total symptom score from baseline (daily average of screening period) to the average daily score for the two weeks after last dose of the anti-Siglec-8 antibody.

### DETAILED DESCRIPTION

### I. Definitions

It is to be understood that the present disclosure is not limited to particular compositions or biological systems, which can, of course, vary. It is also to be understood that the terminology used herein is for the purpose of describing particular embodiments only, and is not intended to be limiting. As used in this specification and the appended claims, the singular forms “a”, “an” and “the” include plural referents unless the content clearly dictates otherwise. Thus, for example, reference to “a molecule” optionally includes a combination of two or more such molecules, and the like.

The term “about” as used herein refers to the usual errr range for the respective value readily known to the skilled person in this technical field. Reference to “about” a value or parameter herein includes (and describes) embodiments that are directed to that value or parameter per se.

It is understood that aspects and embodiments of the present disclosure include “comprising,” “consisting,” and “consisting essentially of” aspects and embodiments.

The term “antibody” includes polyclonal antibodies, monoclonal antibodies (including full length antibodies which have an immunoglobulin Fc region), antibody compositions with polyepitopic specificity, multispecific antibodies (e.g., bispecific antibodies, diabodies, and single-chain molecules), as well as antibody fragments (e.g., Fab, F(ab′)₂, and Fv). The term “immunoglobulin” (Ig) is used interchangeably with “antibody” herein.

The basic 4-chain antibody unit is a heterotetrameric glycoprotein composed of two identical light (L) chains and two identical heavy (H) chains. An IgM antibody consists of 5 of the basic heterotetramer units along with an additional polypeptide called a J chain, and contains 10 antigen binding sites, while IgA antibodies comprise from 2-5 of the basic 4-chain units which can polymerize to form polyvalent assemblages in combination with the J chain. In the case of IgGs, the 4-chain unit is generally about 150,000 daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable domain (V_{H}) followed by three constant domains (C_{H}) for each of the α and γ chains and four C_{H} domains for μ and ε isotypes. Each L chain has at the N-terminus, a variable domain (V_{L}) followed by a constant domain at its other end. The V_{L} is aligned with the V_{H} and the C_{L} is aligned with the first constant domain of the heavy chain (C_{H}1). Particular amino acid residues are believed to form an interface between the light chain and heavy chain variable domains. The pairing of a V_{H} and V_{L} together forms a single antigen-binding site. For the structure and properties of the different classes of antibodies, see e.g., Basic and Clinical Immunology, 8th Edition, Daniel P. Sties, Abba I. Terr and Tristram G. Parsolw (eds), Appleton & Lange, Norwalk, Conn., 1994, page 71 and Chapter 6.

The L chain from any vertebrate species can be assigned to one of two clearly distinct types, called kappa and lambda, based on the amino acid sequences of their constant domains. Depending on the amino acid sequence of the constant domain of their heavy chains (CH), immunoglobulins can be assigned to different classes or isotypes. There are five classes of immunoglobulins: IgA, IgD, IgE, IgG and IgM, having heavy chains designated α, δ, ε, γ and μ, respectively. The γ and α classes are further divided into subclasses on the basis of relatively minor differences in the CH sequence and function, e.g., humans express the following subclasses: IgG1, IgG2, IgG3, IgG4, IgA1 and IgA2. IgG1 antibodies can exist in multiple polymorphic variants termed allotypes (reviewed in Jefferis and Lefranc 2009. mAbs Vol 1 Issue 4 1-7) any of which are suitable for use in the present disclosure. Common allotypic variants in human populations are those designated by the letters a, f, n, z.

An “isolated” antibody is one that has been identified, separated and/or recovered from a component of its production environment (e.g., naturally or recombinantly). In some embodiments, the isolated polypeptide is free of association with all other components from its production environment. Contaminant components of its production environment, such as that resulting from recombinant transfected cells, are materials that would typically interfere with research, diagnostic or therapeutic uses for the antibody, and may include enzymes, hormones, and other proteinaceous or non-proteinaceous solutes. In some embodiments, the polypeptide is purified: (1) to greater than 95% by weight of antibody as determined by, for example, the Lowry method, and in some embodiments, to greater than 99% by weight; (1) to a degree sufficient to obtain at least 15 residues of N-terminal or internal amino acid sequence by use of a spinning cup sequenator, or (3) to homogeneity by SDS-PAGE under non-reducing or reducing conditions using Coomassie blue or silver stain. Isolated antibody includes the antibody in situ within recombinant cells since at least one component of the antibody's natural environment will not be present. Ordinarily, however, an isolated polypeptide or antibody is prepared by at least one purification step.

The term “monoclonal antibody” as used herein refers to an antibody obtained from a population of substantially homogeneous antibodies, i.e., the individual antibodies comprising the population are identical except for possible naturally occurring mutations and/or post-translation modifications (e.g., isomerizations, amidations) that may be present in minor amounts. In some embodiments, monoclonal antibodies have a C-terminal cleavage at the heavy chain and/or light chain. For example, 1, 2, 3, 4, or 5 amino acid residues are cleaved at the C-terminus of heavy chain and/or light chain. In some embodiments, the C-terminal cleavage removes a C-terminal lysine from the heavy chain. In some embodiments, monoclonal antibodies have an N-terminal cleavage at the heavy chain and/or light chain. For example, 1, 2, 3, 4, or 5 amino acid residues are cleaved at the N-terminus of heavy chain and/or light chain. In some embodiments, monoclonal antibodies are highly specific, being directed against a single antigenic site. In some embodiments, monoclonal antibodies are highly specific, being directed against multiple antigenic sites (such as a bispecific antibody or a multispecific antibody). The modifier “monoclonal” indicates the character of the antibody as being obtained from a substantially homogeneous population of antibodies, and is not to be construed as requiring production of the antibody by any particular method. For example, the monoclonal antibodies to be used in accordance with the present disclosure may be made by a variety of techniques, including, for example, the hybridoma method, recombinant DNA methods, phage-display technologies, and technologies for producing human or human-like antibodies in animals that have parts or all of the human immunoglobulin loci or genes encoding human immunoglobulin sequences.

The term “naked antibody” refers to an antibody that is not conjugated to a cytotoxic moiety or radiolabel.

The terms “full-length antibody,” “intact antibody” or “whole antibody” are used interchangeably to refer to an antibody in its substantially intact form, as opposed to an antibody fragment. Specifically whole antibodies include those with heavy and light chains including an Fc region. The constant domains may be native sequence constant domains (e.g., human native sequence constant domains) or amino acid sequence variants thereof. In some cases, the intact antibody may have one or more effector functions.

An “antibody fragment” comprises a portion of an intact antibody, the antigen binding and/or the variable region of the intact antibody. Examples of antibody fragments include Fab, Fab′, F(ab′)₂ and Fv fragments; diabodies; linear antibodies (see U.S. Pat. No. 5,641,870, Example 2: Zapata et al., Protein Eng. 8(10): 1057-1062 [1995]); single-chain antibody molecules and multispecific antibodies formed from antibody fragments.

Papain digestion of antibodies produced two identical antigen-binding fragments, called “Fab” fragments, and a residual “Fc” fragment, a designation reflecting the ability to crystallize readily. The Fab fragment consists of an entire L chain along with the variable region domain of the H chain (V_{H}), and the first constant domain of one heavy chain (C_{H}1). Each Fab fragment is monovalent with respect to antigen binding. i.e., it has a single antigen-binding site. Pepsin treatment of an antibody yields a single large F(ab′)₂ fragment which roughly corresponds to two disulfide linked Fab fragments having different antigen-binding activity and is still capable of cross-linking antigen. Fab′ fragments differ from Fab fragments by having a few additional residues at the carboxy terminus of the CHI domain including one or more cysteines from the antibody hinge region. Fab′-SH is the designation herein for Fab′ in which the cysteine residue(s) of the constant domains bear a free thiol group. F(ab′)2 antibody fragments originally were produced as pairs of Fab′ fragments which have hinge cysteines between them. Other chemical couplings of antibody fragments are also known.

The Fc fragment comprises the carboxy-terminal portions of both H chains held together by disulfides. The effector functions of antibodies are determined by sequences in the Fc region, the region which is also recognized by Fc receptors (FcR) found on certain types of cells.

“Fv” is the minimum antibody fragment which contains a complete antigen-recognition and -binding site. This fragment consists of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three HVRs specific for an antigen) has the ability to recognize and bind antigen, although at a lower affinity than the entire binding site.

“Single-chain Fv” also abbreviated as “sFv” or “scFv” are antibody fragments that comprise the VH and VL antibody domains connected into a single polypeptide chain. In some embodiments, the sFv polypeptide further comprises a polypeptide linker between the V_{H} and V_{L} domains which enables the sFv to form the desired structure for antigen binding. For a review of the sFv, see Pluckthun in The Pharmacology of Monoclonal Antibodies, vol. 113, Rosenburg and Moore eds., Springer-Verlag, New York, pp. 269-315 (1994).

“Functional fragments” of the antibodies of the present disclosure comprise a portion of an intact antibody, generally including the antigen binding or variable region of the intact antibody or the Fv region of an antibody which retains or has modified FcR binding capability. Examples of antibody fragments include linear antibody, single-chain antibody molecules and multispecific antibodies formed from antibody fragments.

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is (are) identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., Proc. Natl. Acad. Sci. USA, 81:6851-6855 (1984)). Chimeric antibodies of interest herein include PRIMATIZED® antibodies wherein the antigen-binding region of the antibody is derived from an antibody produced by, e.g., immunizing macaque monkeys with an antigen of interest. As used herein, “humanized antibody” is used as a subset of “chimeric antibodies.”

“Humanized” forms of non-human (e.g., murine) antibodies are chimeric antibodies that contain minimal sequence derived from non-human immunoglobulin. In one embodiment, a humanized antibody is a human immunoglobulin (recipient antibody) in which residues from an HVR of the recipient are replaced by residues from an HVR of a non-human species (donor antibody) such as mouse, rat, rabbit or non-human primate having the desired specificity, affinity, and/or capacity. In some instances, FR residues of the human immunoglobulin are replaced by corresponding non-human residues. Furthermore, humanized antibodies may comprise residues that are not found in the recipient antibody or in the donor antibody. These modifications may be made to further refine antibody performance, such as binding affinity. In general, a humanized antibody will comprise substantially all of at least one, and typically two, variable domains, in which all or substantially all of the hypervariable loops correspond to those of a non-human immunoglobulin sequence, and all or substantially all of the FR regions are those of a human immunoglobulin sequence, although the FR regions may include one or more individual FR residue substitutions that improve antibody performance, such as binding affinity, isomerization, immunogenicity, etc. In some embodiments, the number of these amino acid substitutions in the FR are no more than 6 in the H chain, and in the L chain, no more than 3. The humanized antibody optionally will also comprise at least a portion of an immunoglobulin constant region (Fc), typically that of a human immunoglobulin. For further details, see, e.g., Jones et al., Nature 321:522-525 (1986); Riechmann et al., Nature 332:323-329 (1988); and Presta, Curr. Op. Struct. Biol. 2:593-596 (1992). See also, for example, Vaswani and Hamilton, Ann. Allergy, Asthma & Immunol. 1:105-115 (1998): Harris, Biochem. Soc. Transactions 23:1035-1038 (1995); Hurle and Gross, Curr. Op. Biotech. 5:428-433 (1994); and U.S. Pat. Nos. 6,982,321 and 7,087,409. In some embodiments, humanized antibodies are directed against a single antigenic site. In some embodiments, humanized antibodies are directed against multiple antigenic sites. An alternative humanization method is described in U.S. Pat. No. 7,981,843 and U.S. Patent Application Publication No. 2006/0134098.

The “variable region” or “variable domain” of an antibody refers to the amino-terminal domains of the heavy or light chain of the antibody. The variable domains of the heavy chain and light chain may be referred to as “VH” and “VL”, respectively. These domains are generally the most variable parts of the antibody (relative to other antibodies of the same class) and contain the antigen binding sites.

The term “hypervariable region,” “HVR,” or “HV,” when used herein refers to the regions of an antibody-variable domain that are hypervariable in sequence and/or form structurally defined loops. Generally, antibodies comprise six HVRs; three in the VH (H1, H2, H3), and three in the VL (L1, L2, L3). In native antibodies, H3 and L3 display the most diversity of the six HVRs. and H3 in particular is believed to play a unique role in conferring fine specificity to antibodies. See, e.g., Xu et al. Immunity 13:3745 (2000); Johnson and Wu in Methods in Molecular Biology 248:1-25 (Lo, ed., Human Press, Totowa, N.J., 2003)). Indeed, naturally occurring camelid antibodies consisting of a heavy chain only are functional and stable in the absence of light chain. See, e.g., Hamers-Casterman et al., Nature 363:446-448 (1993) and Sheriff et al., Nature Struct. Biol. 3:733-736 (1996).

A number of HVR delineations are in use and are encompassed herein. The HVRs that are Kabat complementarity-determining regions (CDRs) are based on sequence variability and are the most commonly used (Kabat et al., Sequences of Proteins of Immunological Interest. 5^{th} Ed. Public Health Service, National Institute of Health, Bethesda, Md. (1991)). Chothia HVRs refer instead to the location of the structural loops (Chothia and Lesk J. Mol. Biol. 196:901-917 (1987)). The “contact” HVRs are based on an analysis of the available complex crystal structures. The residues from each of these HVRs are noted below.

| Loop | Kabat | Chothia | Contact |
|---|---|---|---|
| L1 | L24-L34 | L26-L34 | L30-L36 |
| L2 | L50-L56 | L50-L56 | L46-L55 |
| L3 | L89-L97 | L91-L96 | L89-L96 |
| H1 | H31-H35B | H26-H32 | H30-H35B (Kabat Numbering) |
| H1 | H31-H35 | H26-H32 | H30-H35 (Chothia Numbering) |
| H2 | H50-H65 | H53-H56 | H47-H58 |
| H3 | H95-H102 | H95-H102 | H93-H101 |

Unless otherwise indicated, the variable-domain residues (HVR residues and framework region residues) are numbered according to Kabat et al., supra.

“Framework” or “FR” residues are those variable-domain residues other than the HVR residues as herein defined.

The expression “variable-domain residue-numbering as in Kabat” or “amino-acid-position numbering as in Kabat,” and variations thereof, refers to the numbering system used for heavy-chain variable domains or light-chain variable domains of the compilation of antibodies in Kabat et al., supra. Using this numbering system, the actual linear amino acid sequence may contain fewer or additional amino acids corresponding to a shortening of, or insertion into, a FR or HVR of the variable domain. For example, a heavy-chain variable domain may include a single amino acid insert (residue 52a according to Kabat) after residue 52 of H2 and inserted residues (e.g. residues 82a, 82b, and 82c, etc. according to Kabat) after heavy-chain FR residue 82. The Kabat numbering of residues may be determined for a given antibody by alignment at regions of homology of the sequence of the antibody with a “standard” Kabat numbered sequence.

An “acceptor human framework” for the purposes herein is a framework comprising the amino acid sequence of a VL or VH framework derived from a human immunoglobulin framework or a human consensus framework. An acceptor human framework “derived from” a human immunoglobulin framework or a human consensus framework may comprise the same amino acid sequence thereof, or it may contain pre-existing amino acid sequence changes. In some embodiments, the number of pre-existing amino acid changes are 10 or less, 9 or less, 8 or less, 7 or less, 6 or less, 5 or less, 4 or less, 3 or less, or 2 or less.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For example, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A.

An antibody that “binds to”, “specifically binds to” or is “specific for” a particular a polypeptide or an epitope on a particular polypeptide is one that binds to that particular polypeptide or epitope on a particular polypeptide without substantially binding to any other polypeptide or polypeptide epitope. In some embodiments, binding of an anti-Siglec-8 antibody described herein (e.g., an antibody that binds to human Siglec-8) to an unrelated non-Siglec-8 polypeptide is less than about 10% of the antibody binding to Siglec-8 as measured by methods known in the art (e.g., enzyme-linked immunosorbent assay (ELISA)). In some embodiments, an antibody that binds to a Siglec-8 (e.g., an antibody that binds to human Siglec-8) has a dissociation constant (Kd) of ≤1 μM, ≤100 nM, ≤10 nM, ≤2 nM, ≤1 nM, ≤0.7 nM, ≤0.6 nM, ≤0.5 nM, ≤0.1 nM, ≤0.01 nM, or ≤0.001 nM (e.g. 10⁻⁸ M or less, e.g. from 10⁻⁸ M to 10⁻¹³ M. e.g., from 10⁻⁹ M to 10⁻¹³ M).

The term “anti-Siglec-8 antibody” or “an antibody that binds to human Siglec-8” refers to an antibody that binds to a polypeptide or an epitope of human Siglec-8 without substantially binding to any other polypeptide or epitope of an unrelated non-Siglec-8 polypeptide.

The term “Siglec-8” as used herein refers to a human Siglec-8 protein. The term also includes naturally occurring variants of Siglec-8, including splice variants or allelic variants. The amino acid sequence of an exemplary human Siglec-8 is shown in SEQ ID NO:72. The amino acid sequence of another exemplary human Siglec-8 is shown in SEQ ID NO:73. In some embodiments, a human Siglec-8 protein comprises the human Siglec-8 extracellular domain fused to an immunoglobulin Fc region. The amino acid sequence of an exemplary human Siglec-8 extracellular domain fused to an immunoglobulin Fc region is shown in SEQ ID NO:74. The amino acid sequence underlined in SEQ ID NO:74 indicates the Fc region of the Siglec-8 Fc fusion protein amino acid sequence.

Human Siglec-8 Amino Acid Sequence
(SEQ ID NO: 72)
GYLLQVQELVTVQEGLCVHVPCSFSYPQDGWTDSDPVHGYWFRAGDRPYQD
APVATNNPDREVQAETQGRFQLLGDIWSNDCSLSIRDARKRDKGSYFFRLE
RGSMKNVSYKSQLNYKTKQLSVFVTALTHRPDILILGTESGHSRNLTCSVP
WACKQGTPPMISWIGASVSSPGPTTARSSVLTLTPKPQDHGTSTCQVTLPG
TGVTTTSTVRLDVSYPPWNLTMTVFQGDATASTALGNGSSLSVLEGQSLRL
VCAVNSNPPARLSWTRGSLTLCPSRSSNPGLLELPRVHVRDEGEFTCRAQN
AQGSQHISLSLSLQNEGTGTSRPVSQVTLAAVGGAGATALAFLSFCIIFII
VRSCRKKSARPAAGVGDTGMEDAKAIRGSASQGPLTESWKDGNPLKKPPPA
VAPSSGEEGELHYATLSFHKVKPQDPQGQEATDSEYSEIKIHKRETAETQA
CLRNHNPSSKEVRG
Human Siglec-8 Amino Acid Sequence
(SEQ ID NO: 73)
GYLLQVQELVTVQEGLCVHVPCSFSYPQDGWTDSDPVHGYWFRAGDRPYQD
APVATNNPDREVQAETQGRFQLLGDIWSNDCSLSIRDARKRDKGSYFFRLE
RGSMKWSYKSQLNYKTKQLSVFVTALTHRPDILILGTLESGHPRNLTCSVP
WACKQGTPPMISWIGASVSSPGPTTARSSVLTLTPKPQDHGTSLTCQVTLP
GTGVTTTSTVRLDVSYPPWNLTMTVFQGDATASTALGNGSSLSVLEGQSLR
LVCAVNSNPPARLSWTRGSLTLCPSRSSNPGLLELPRVHVRDEGEFTCRAQ
NAQGSQHISLSLSLQNEGTGTSRPVSQVTLAAVGGAGATALAFLSFCIIFI
IVRSCRKKSARPAAGVGDTGMEDAKAIRGSASQGPLTESWKDGNPLKKPPP
AVAPSSGEEGELHYATLSFHKVKPQDPQGQEATDSEYSEIKIHKRETAETQ
ACLRNHNPSSKEVRG
Siglec-8 Fc Fusion Protein Amino Acid Sequence
(SEQ ID NO: 74)
GYLLQVQELVTVQEGLCVHVPCSFSYPQDGWTDSDPVHGYWFRAGDRPYQD
APVATNNPDREVQAETQGRFQLLGDIWSNDCSLSIRDARKRDKGSYFFRLE
RGSMKWSYKSQLNYKTKQLSVFVTALTHRPDILILGTLESGHSRNLTCSVP
WACKQGTPPMISWIGASVSSPGPTTARSSVLTLTPKPQDHGTSLTCQVTLP
GTGVTTTSTVRLDVSYPPWNLTMTVFQGDATASTALGNGSSLSVLEGQSLR
LVCAVNSNPPARLSWTRGSLTLCPSRSSNPGLLELPRVHVRDEGEFTCRAQ
NAQGSQHISLSLSLQNEGTGTSRPVSQVTLAAVGGIEGRSDKTHTCPPCPA
PELLGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPAPIE
KTISKAKGQPREPQVYTLPPSREEMTKNQVSLTCLVKGFYPSDIAVEWESN
GQPENNYKTTPPVLPSPGSFFLYSKLTVDKSRWQQGNVFSCSVMHEALHNH
YTQKSLSLSPGK

Antibodies that “induce apoptosis” or are “apoptotic” are those that induce programmed cell death as determined by standard apoptosis assays, such as binding of annexin V, fragmentation of DNA, cell shrinkage, dilation of endoplasmic reticulum, cell fragmentation, and/or formation of membrane vesicles (called apoptotic bodies). For example, the apoptotic activity of the anti-Siglec-8 antibodies (e.g., an antibody that binds to human Siglec-8) of the present disclosure can be shown by staining cells with annexin V.

Antibody “effector functions” refer to those biological activities attributable to the Fc region (a native sequence Fc region or amino acid sequence variant Fc region) of an antibody, and vary with the antibody isotype. Examples of antibody effector functions include: C1q binding and complement dependent cytotoxicity; Fc receptor binding; antibody-dependent cell-mediated cytotoxicity (ADCC); phagocytosis; down regulation of cell surface receptors (e.g., B cell receptors); and B cell activation.

“Antibody-dependent cell-mediated cytotoxicity” or “ADCC” refers to a form of cytotoxicity in which secreted Ig bound onto Fc receptors (FcRs) present on certain cytotoxic cells (e.g., natural killer (NK) cells, neutrophils and macrophages) enable these cytotoxic effector cells to bind specifically to an antigen-bearing target cell and subsequently kill the target cell with cytotoxins. The antibodies “arm” the cytotoxic cells and are required for killing of the target cell by this mechanism. The primary cells for mediating ADCC, NK cells, express FcγRIII only, whereas monocytes express FcγRI, FcγRII and FcγRIII. Fc expression on hematopoietic cells is summarized in Table 3 on page 464 of Ravetch and Kinet, Annu. Rev. Immunol. 9: 457-92 (1991). In some embodiments, an anti-Siglec-8 antibody (e.g., an antibody that binds to human Siglec-8) described herein enhances ADCC. To assess ADCC activity of a molecule of interest, an in vitro ADCC assay, such as that described in U.S. Pat. No. 5,500,362 or 5,821,337 may be performed. Useful effector cells for such assays include peripheral blood mononuclear cells (PBMC) and natural killer (NK) cells. Alternatively, or additionally, ADCC activity of the molecule of interest may be assessed in vivo, e.g., in an animal model such as that disclosed in Clynes et al., PNAS USA 95:652-656 (1998). Other Fc variants that alter ADCC activity and other antibody properties include those disclosed by Ghetie et al., Nat Biotech. 15:637-40, 1997; Duncan et al, Nature 332:563-564, 1988: Lund et al., J. Immunol 147:2657-2662, 1991; Lund et al, Mol Immunol 29:53-59, 1992: Alegre et al, Transplantation 57:1537-1543, 1994; Hutchins et al., Proc Natl. Acad Sci USA 92:11980-11984, 1995; Jefferis et al, Immunol Lett. 44:111-117, 1995; Lund et al., FASEB J9:115-119, 1995; Jefferis et al, Immunol Lett 54:101-104, 1996; Lund ct al, J Immunol 157:49634969, 1996: Armour et al., Eur J Immunol 29:2613-2624, 1999; Idusogie et al, J Immunol 164:4178-4184, 200; Reddy et al, J Immunol 164:1925-1933, 2000; Xu et al., Cell Immunol 200:16-26, 2000; Idusogie et al, J Immunol 166:2571-2575, 2001; Shields et al., J Biol Chem 276:6591-6604, 2001; Jefferis et al, Immunol Lett 82:57-65. 2002; Presta et al., Biochem Soc Trans 30:487-490, 2002; Lazar ct al., Proc. Natl. Acad. Sci. USA 103:4005-4010, 2006: U.S. Pat. Nos. 5,624,821; 5,885,573; 5,677,425; 6,165,745; 6,277,375; 5,869,046; 6,121,022; 5,624,821; 5,648,260; 6,194,551; 6,737,056; 6,821,505; 6,277,375; 7.335,742; and 7,317,091.

The term “Fc region” herein is used to define a C-terminal region of an immunoglobulin heavy chain, including native-sequence Fc regions and variant Fc regions. Although the boundaries of the Fc region of an immunoglobulin heavy chain might vary, the human IgG heavy-chain Fc region is usually defined to stretch from an amino acid residue at position Cys226, or from Pro230, to the carboxyl-terminus thereof. Suitable native-sequence Fc regions for use in the antibodies of the present disclosure include human IgG1, IgG2, IgG3 and IgG4. A single amino acid substitution (S228P according to Kabat numbering; designated IgG4Pro) may be introduced to abolish the heterogeneity observed in recombinant IgG4 antibody. See Angal, S. et al. (1993) Mol Immunol 30, 105-108.

“Non-fucosylated” or “fucose-deficient” antibody refers to a glycosylation antibody variant comprising an Fc region wherein a carbohydrate structure attached to the Fc region has reduced fucose or lacks fucose. In some embodiments, an antibody with reduced fucose or lacking fucose has improved ADCC function. Non-fucosylated or fucose-deficient antibodies have reduced fucose relative to the amount of fucose on the same antibody produced in a cell line. In some embodiments, a non-fucosylated or fucose-deficient antibody composition contemplated herein is a composition wherein less than about 50% of the N-linked glycans attached to the Fc region of the antibodies in the composition comprise fucose.

The terms “fucosylation” or “fucosylated” refers to the presence of fucose residues within the oligosaccharides attached to the peptide backbone of an antibody. Specifically, a fucosylated antibody comprises α (1,6)-linked fucose at the innermost N-acetylglucosamine (GlcNAc) residue in one or both of the N-linked oligosaccharides attached to the antibody Fc region, e.g. at position Asn 297 of the human IgG1 Fc domain (EU numbering of Fc region residues). Asn297 may also be located about +3 amino acids upstream or downstream of position 297, i.e. between positions 294 and 300, due to minor sequence variations in immunoglobulins.

The “degree of fucosylation” is the percentage of fucosylated oligosaccharides relative to all oligosaccharides identified by methods known in the art e.g., in an N-glycosidase F treated antibody composition assessed by matrix-assisted laser desorption-ionization time-of-flight mass spectrometry (MALDI-TOF MS). In a composition of a “fully fucosylated antibody” essentially all oligosaccharides comprise fucose residues, i.e. are fucosylated. In some embodiments, a composition of a fully fucosylated antibody has a degree of fucosylation of at least about 90%. Accordingly, an individual antibody in such a composition typically comprises fucose residues in each of the two N-linked oligosaccharides in the Fc region. Conversely, in a composition of a “fully non-fucosylated” antibody essentially none of the oligosaccharides are fucosylated, and an individual antibody in such a composition does not contain fucose residues in either of the two N-linked oligosaccharides in the Fc region. In some embodiments, a composition of a fully non-fucosylated antibody has a degree of fucosylation of less than about 10%. In a composition of a “partially fucosylated antibody” only part of the oligosaccharides comprise fucose. An individual antibody in such a composition can comprise fucose residues in none, one or both of the N-linked oligosaccharides in the Fc region, provided that the composition does not comprise essentially all individual antibodies that lack fucose residues in the N-linked oligosaccharides in the Fc region, nor essentially all individual antibodies that contain fucose residues in both of the N-linked oligosaccharides in the Fc region. In one embodiment, a composition of a partially fucosylated antibody has a degree of fucosylation of about 10% to about 80% (e.g., about 50% to about 80%, about 60% to about 80%, or about 70% to about 80%).

“Binding affinity” as used herein refers to the strength of the non-covalent interactions between a single binding site of a molecule (e.g., an antibody) and its binding partner (e.g., an antigen). In some embodiments, the binding affinity of an antibody for a Siglec-8 (which may be a dimer, such as the Siglec-8-Fc fusion protein described herein) can generally be represented by a dissociation constant (Kd). Affinity can be measured by common methods known in the art, including those described herein.

“Binding avidity” as used herein refers to the binding strength of multiple binding sites of a molecule (e.g., an antibody) and its binding partner (e.g., an antigen).

An “isolated” nucleic acid molecule encoding the antibodies herein is a nucleic acid molecule that is identified and separated from at least one contaminant nucleic acid molecule with which it is ordinarily associated in the environment in which it was produced. In some embodiments, the isolated nucleic acid is free of association with all components associated with the production environment. The isolated nucleic acid molecules encoding the polypeptides and antibodies herein is in a form other than in the form or setting in which it is found in nature. Isolated nucleic acid molecules therefore are distinguished from nucleic acid encoding the polypeptides and antibodies herein existing naturally in cells.

The term “pharmaceutical formulation” refers to a preparation that is in such form as to permit the biological activity of the active ingredient to be effective, and that contains no additional components that are unacceptably toxic to an individual to which the formulation would be administered. Such formulations are sterile.

“Carriers” as used herein include pharmaceutically acceptable carriers, excipients, or stabilizers that are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol, salt-forming counterions such as sodium; and/or nonionic surfactants such as TWEEN™, polyethylene glycol (PEG), and PLURONICS™.

As used herein, the term “treatment” or “treating” refers to clinical intervention designed to alter the natural course of the individual or cell being treated during the course of clinical pathology. Desirable effects of treatment include decreasing the rate of disease progression, ameliorating or palliating the disease state, and remission or improved prognosis. An individual is successfully “treated”, for example, if one or more symptoms associated with a disease (e.g., mast cell gastritis, mast cell esophagitis, mast cell colitis, mast cell enteritis, and/or mast cell gastroenteritis) are mitigated or eliminated. For example, an individual is successfully “treated” if treatment results in increasing the quality of life of those suffering from a disease, decreasing the dose of other medications required for treating the disease, reducing the frequency of recurrence of the disease, lessening severity of the disease, delaying the development or progression of the disease, and/or prolonging survival of individuals.

As used herein, “in conjunction with” or “in combination with” refers to administration of one treatment modality in addition to another treatment modality. As such, “in conjunction with” or “in combination with” refers to administration of one treatment modality before, during or after administration of the other treatment modality to the individual.

As used herein, the term “prevention” or “preventing” includes providing prophylaxis with respect to occurrence or recurrence of a disease in an individual. An individual may be predisposed to a disease, susceptible to a disease, or at risk of developing a disease, but has not yet been diagnosed with the disease. In some embodiments, anti-Siglec-8 antibodies (e.g., an antibody that binds to human Siglec-8) described herein are used to delay development of a disease (e.g., mast cell gastritis, mast cell esophagitis, mast cell colitis, mast cell enteritis, and/or mast cell gastroenteritis).

As used herein, an individual “at risk” of developing a disease (e.g., mast cell gastritis, mast cell esophagitis, mast cell colitis, mast cell enteritis, and/or mast cell gastroenteritis) may or may not have detectable disease or symptoms of disease, and may or may not have displayed detectable disease or symptoms of disease prior to the treatment methods described herein. “At risk” denotes that an individual has one or more risk factors, which are measurable parameters that correlate with development of the disease (e.g., mast cell gastritis, mast cell esophagitis, mast cell colitis, mast cell enteritis, and/or mast cell gastroenteritis), as known in the art. An individual having one or more of these risk factors has a higher probability of developing the disease than an individual without one or more of these risk factors.

An “effective amount” refers to at least an amount effective, at dosages and for periods of time necessary, to achieve the desired or indicated effect, including a therapeutic or prophylactic result. An effective amount can be provided in one or more administrations. A “therapeutically effective amount” is at least the minimum concentration required to effect a measurable improvement of a particular disease. A therapeutically effective amount herein may vary according to factors such as the disease state, age, sex, and weight of the patient, and the ability of the antibody to elicit a desired response in the individual. A therapeutically effective amount may also be one in which any toxic or detrimental effects of the antibody are outweighed by the therapeutically beneficial effects. A “prophylactically effective amount” refers to an amount effective, at the dosages and for periods of time necessary, to achieve the desired prophylactic result. Typically but not necessarily, since a prophylactic dose is used in individuals prior to or at the earlier stage of disease, the prophylactically effective amount can be less than the therapeutically effective amount.

“Chronic” administration refers to administration of the medicament(s) in a continuous as opposed to acute mode, so as to maintain the initial therapeutic effect (activity) for an extended period of time. “Intermittent” administration is treatment that is not consecutively done without interruption, but rather is cyclic in nature.

The term “package insert” is used to refer to instructions customarily included in commercial packages of therapeutic products, that contain information about the indications, usage, dosage, administration, combination therapy, contraindications and/or warnings concerning the use of such therapeutic products.

As used herein, an “individual” or a “subject” is a mammal. A “mammal” for purposes of treatment includes humans, domestic and farm animals, and zoo, sports, or pet animals, such as dogs, horses, rabbits, cattle, pigs, hamsters, gerbils, mice, ferrets, rats, cats, etc. In some embodiments, the individual or subject is a human.

### II. Methods

Provided herein are methods for treating and/or preventing mast cell gastritis, mast cell esophagitis, mast cell enteritis, mast cell colitis, and/or mast cell gastroenteritis in an individual comprising administering to the individual an effective amount of an antibody described herein that binds to human Siglec-8 (e.g., an anti-Siglec-8 antibody) or compositions comprising said antibodies. In some embodiments, the antibody is in a pharmaceutical composition comprising the antibody and a pharmaceutically acceptable carrier. In some embodiments, the individual is a human. In some embodiments, the individual has mast cell gastritis. In some embodiments, the individual has mast cell esophagitis. In some embodiments, the individual has mast cell enteritis. In some embodiments, the individual has mast cell gastroenteritis. In some embodiments, the individual has mast cell gastritis and mast cell gastroenteritis. In some embodiments, the individual has mast cell gastritis and mast cell enteritis. In some embodiments, the individual has mast cell esophagitis and mast cell gastritis. In some embodiments, the individual has mast cell esophagitis and mast cell gastroenteritis. In some embodiments, the individual has mast cell esophagitis and mast cell enteritis. In some embodiments, the individual has mast cell esophagitis, mast cell gastritis, and mast cell gastroenteritis. In some embodiments, the individual has mast cell esophagitis, mast cell gastritis, and mast cell enteritis.

In some embodiments, the methods comprise detecting a number of mast cells and a number of eosinophils from one or more samples obtained from the gastric, duodenal, jejunal, ileal, or colonic mucosa of the individual (e.g., for mast cell gastritis or mast cell gastroenteritis). In some embodiments, the methods comprise detecting a number of mast cells and a number of eosinophils from one or more samples obtained from the esophageal mucosa of the individual (e.g., for mast cell esophagitis).

A. Mast Cell Gastritis, Mast Cell Esophagitis, Mast Cell Colitis, Mast Cell Enteritis, Mast Cell Duodenitis, and/or Mast Cell Gastroenteritis

Certain aspects of the present disclosure relate to individuals with mast cell gastritis, mast cell esophagitis, mast cell colitis, mast cell enteritis, mast cell duodenitis, and/or mast cell gastroenteritis. The present disclosure is based at least in part on the finding that a subpopulation of clinical study patients, despite having symptoms of eosinophilic gastritis/gastroenteritis, did not have the increased number of eosinophils in the gastric and/or duodenal mucosa typically used to diagnose eosinophilic gastritis/gastroenteritis. Instead, it was found that these patients had a substantial number of mast cells (in most cases greater than 30 mast cells/high power field (HPF)) in the stomach and/or duodenal mucosa. Normal levels have been measured to be approximately less than 20 mast cells/HPF (Doyle et al., Am. J. Surg. Pathol. (2014) 38:832-843, Jakate et al., Arch. Pathol. Lab. Med. (2006) 130:362-367; Tison et al., Allergy Clin. Immunol. (2010) Abstract 714), implying that the elevated mast cells in these patients may be responsible for the gastrointestinal symptomatology.

In some embodiments, the individual has, or has been diagnosed with, gastroesophageal reflux disease (GERD) (e.g., prior to treatment with an anti-Siglec-8 antibody). In some embodiments, the individual has, or has been diagnosed with, gastroesophageal reflux disease (GERD) (e.g., prior to treatment with an anti-Siglec-8 antibody) and is refractory to treatment with an antacid, H2 blocker, and/or proton pump inhibitor. For example, one or more GERD symptoms in the individual may be refractory to treatment with an antacid, H2 blocker, and/or proton pump inhibitor, or the individual may have GERD that is refractory to treatment with an antacid, H2 blocker, and/or proton pump inhibitor. In some embodiments, the individual has, or has been diagnosed with, irritable bowel syndrome (IBS) (e.g., prior to treatment with an anti-Siglec-8 antibody). In some embodiments, the individual previously had, or was diagnosed with (e.g., has a previous history of), eosinophilic gastritis, but is symptomatic without elevated eosinophils (e.g., prior to treatment with an anti-Siglec-8 antibody). In some embodiments, the individual has had (e.g., prior to treatment with an anti-Siglec-8 antibody), or has previously been diagnosed with (e.g., prior to treatment with an anti-Siglec-8 antibody), one or more of: abdominal pain, abdominal cramping, nausea, vomiting, diarrhea, bloating, and early satiety without identifiable cause. In some embodiments, the individual is (e.g., prior to treatment with an anti-Siglec-8 antibody) refractory and/or unresponsive to pharmacologic and/or dietary intervention. In some embodiments, the individual has had (e.g., prior to treatment with an anti-Siglec-8 antibody), or has previously been diagnosed with (e.g., prior to treatment with an anti-Siglec-8 antibody), eosinophilic gastritis, and the individual has one or more symptoms of eosinophilic gastritis (e.g., is symptomatic) without elevated eosinophils. In some embodiments, the individual previously had, or was diagnosed with (e.g., has a previous history of), eosinophilic gastroenteritis, but is symptomatic without elevated eosinophils (e.g., prior to treatment with an anti-Siglec-8 antibody). In some embodiments, the individual has had (e.g., prior to treatment with an anti-Siglec-8 antibody), or has previously been diagnosed with (e.g., prior to treatment with an anti-Siglec-8 antibody), eosinophilic gastroenteritis, and the individual has one or more symptoms of eosinophilic gastroenteritis (e.g., is symptomatic) without elevated eosinophils. For example, prior to treatment with an anti-Siglec-8 antibody, the individual may not present with elevated eosinophils (e.g., from a biopsy as described herein), but may have had a previous history or diagnosis of eosinophilic gastritis or eosinophilic gastroenteritis. In some embodiments, the individual has, or has been diagnosed with, functional dyspepsia (e.g., prior to treatment with an anti-Siglec-8 antibody). Without wishing to be bound to theory, it is thought that elevated mast cells may be responsible for symptomatology in disease states such as GERD, IBS, and functional dyspepsia. e.g., having symptoms resembling eosinophilic gastritis/gastroenteritis even if the number of eosinophils in the gastric and/or duodenal mucosa (e.g., from a biopsy as discussed herein) does not meet clinical standards for eosinophilic disease/involvement.

In some embodiments, the individual has an increased number of mast cells (e.g., as compared to a mast cell reference) in at least a portion of the gastric, duodenal, jejunal, ileal, or colonic mucosa, but does not have an increased number of eosinophils (e.g., as compared to an eosinophil reference) in at least a portion of the gastric, duodenal, jejunal, ileal, or colonic mucosa (e.g., for mast cell gastritis or mast cell gastroenteritis). In some embodiments, the individual has an increased number of mast cells (e.g., as compared to a mast cell reference) in at least a portion of the esophageal mucosa, but does not have an increased number of eosinophils (e.g., as compared to an eosinophil reference) in at least a portion of the esophageal mucosa (e.g., for mast cell esophagitis).

In some embodiments, a sample obtained from the gastric, duodenal, jejunal, ileal, or colonic mucosa of the individual has an increased number of mast cells, e.g., as compared to a mast cell reference, but a sample obtained from the gastric, duodenal, jejunal, ileal, or colonic mucosa of the individual does not have increased number of eosinophils, e.g., as compared to an eosinophil reference (e.g., for mast cell gastritis or mast cell gastroenteritis). In some embodiments, the samples are the same sample. In some embodiments, the samples are different samples. In some embodiments, the samples are from the same type of tissue. In some embodiments, a sample obtained from the esophageal mucosa of the individual has an increased number of mast cells, e.g., as compared to a mast cell reference, but a sample obtained from the esophageal mucosa of the individual does not have increased number of eosinophils, e.g., as compared to an eosinophil reference (e.g., for mast cell esophagitis). In some embodiments, the samples are the same sample. In some embodiments, the samples are different samples.

In some embodiments, a sample from the gastric, duodenal, jejunal, ileal, or colonic mucosa has at least one, at least two, at least three, at least four, or at least five high-power fields (HPFs) that each have a mast cell count of 30 or more mast cells per HPF. In some embodiments, a sample from the esophageal mucosa has at least one, two, three, four, or five high-power fields (HPFs) that each have a mast cell count of 10 or more mast cells per HPF. In some embodiments, a sample from the esophageal mucosa has at least one, two, three, four, or five high-power fields (HPFs) that each have a mast cell count of 20 or more mast cells per HPF. In some embodiments, a sample from the esophageal mucosa has at least one, two, three, four, or five high-power fields (HPFs) that each have a mast cell count of 25 or more mast cells per HPF. In some embodiments, the peak mast cell count obtained from two or more HPFs from a sample from the esophageal mucosa is 10 or more mast cells per HPF. In some embodiments, the peak mast cell count obtained from two or more HPFs from a sample from the esophageal mucosa is 15 or more mast cells per HPF. In some embodiments, an individual is selected for treatment (e.g., using any of the methods of the present disclosure) if a sample (e.g., biopsy sample) obtained from the individual has at least one, two, three, four, or five high-power fields (HPFs) that each have a mast cell count of 10 or more mast cells per HPF (for a sample from the esophageal mucosa). In some embodiments, an individual is selected for treatment (e.g., using any of the methods of the present disclosure) if a sample (e.g., biopsy sample) obtained from the individual has at least one, two, three, four, or five high-power fields (HPFs) that each have a mast cell count of 30 or more mast cells per HPF (for a sample from the gastric, duodenal, jejunal, or ileal mucosa). In some embodiments, an individual is selected for treatment (e.g., using any of the methods of the present disclosure) if a sample (e.g., biopsy sample) obtained from the individual has at least one, two, three, four, or five high-power fields (HPFs) that each have a mast cell count of 10 or more mast cells per HPF (for a sample from the esophageal mucosa). In some embodiments, an individual is selected for treatment (e.g., using any of the methods of the present disclosure) if a sample (e.g., biopsy sample) obtained from the individual has at least one, two, three, four, or five high-power fields (HPFs) that each have a mast cell count of 20 or more mast cells per HPF (for a sample from the gastric, duodenal, jejunal, or ileal mucosa). In some embodiments, an individual is selected for treatment (e.g., using any of the methods of the present disclosure) if a sample (e.g., biopsy sample) obtained from the individual has at least one, two, three, four, or five high-power fields (HPFs) that each have a mast cell count of 20-30 mast cells per HPF (for a sample from the gastric, duodenal, jejunal, or ileal mucosa).

In some embodiments, number of mast cells in a sample is detected less than about 14, less than about 28, less than about 35, less than about 45, or less than about 90 days prior to administration of a composition or anti-Siglec-8 antibody of the present disclosure.

In some embodiments, a sample from the gastric, duodenal, jejunal, or ileal mucosa has one or more HPFs that each have an eosinophil count of less than 30 eosinophils per HPF. In some embodiments, a sample from the gastric, duodenal, jejunal, or ileal mucosa does not have at least one, at least two, at least three, four, or five HPFs that each have an eosinophil count of 30 or more eosinophils per HPF. In some embodiments, a sample from the gastric mucosa does not have at least five HPFs that each have an eosinophil count of 30 or more eosinophils per HPF. In some embodiments, a sample from the duodenal, jejunal, or ileal mucosa does not have at least three HPFs that each have an eosinophil count of 30 or more eosinophils per HPF. In some embodiments, an individual is selected for treatment (e.g., using any of the methods of the present disclosure) if a sample (e.g., biopsy sample) obtained from the individual has at least one, at least two, at least three, at least four, or at least five high-power fields (HPFs) that each have a mast cell count of 30 or more mast cells per HPF (for a sample from the gastric, duodenal, jejunal, or ileal mucosa) and if a sample (e.g., biopsy sample) obtained from the individual does not have increased eosinophils, e.g., if a sample from the gastric, duodenal, jejunal, or ileal mucosa does not have at least one, at least two, at least three, at least four, or at least five HPFs that each have an eosinophil count of 30 or more eosinophils per HPF (e.g., if a sample from the gastric, duodenal, jejunal, or ileal mucosa does not have at least one, at least two, at least three, at least four, or at least five HPFs that each have an eosinophil count of 30 or more eosinophils per HPF or if a sample from the gastric, duodenal, jejunal, or ileal mucosa does not have at least five HPFs that each have an eosinophil count of 30 or more eosinophils per HPF). For example, the eosinophil sample may have 1, 2, 3, 4, or 5 HPFs, or all HPFs tested, may fail to meet clinical criteria for eosinophilic disease.

In some embodiments, a sample from the colonic mucosa has one or more HPFs that each have an eosinophil count of less than 60 eosinophils per HPF. In some embodiments, a sample from the colonic mucosa does not have at least one, at least two, at least three, at least four, or at least five HPFs that each have an eosinophil count of 60 or more eosinophils per HPF. In some embodiments, a sample from the colonic mucosa does not have at least five HPFs that each have an eosinophil count of 60 or more eosinophils per HPF. In some embodiments, a sample from the colonic mucosa does not have at least three HPFs that each have an eosinophil count of 60 or more eosinophils per HPF. In some embodiments, a sample from the colonic mucosa does not have at least two HPFs that each have an eosinophil count of 60 or more eosinophils per HPF. In some embodiments, a sample from the colonic mucosa does not have an HPF that has an eosinophil count of 60 or more eosinophils. In some embodiments, an individual is selected for treatment (e.g., using any of the methods of the present disclosure) if a sample (e.g., biopsy sample) obtained from the individual has at least one, two, three, four, or five high-power fields (HPFs) that each have a mast cell count of 20, 30, or more mast cells per HPF (for a sample from the colonic mucosa) and if a sample (e.g., biopsy sample) obtained from the individual does not have increased eosinophils, e.g., if a sample from the colonic mucosa does not have at least one, at least two, at least three, at least four, or at least five HPFs that each have an eosinophil count of 60 or more eosinophils per HPF (e.g., if a sample from the colonic mucosa does not have at least one, two, three, four, or five HPFs that each have an eosinophil count of 60 or more eosinophils per HPF). For example, the eosinophil sample may have 1, 2, 3, 4, or 5 HPFs, or all HPFs tested, may fail to meet clinical criteria for eosinophilic disease.

In some embodiments, a sample from the esophageal mucosa has one or more HPFs that each have an eosinophil count of less than 10 eosinophils per HPF. In some embodiments, a sample from the esophageal mucosa does not have at least one, at least two, at least three, four, or five HPFs that each have an eosinophil count of 10 or more eosinophils per HPF. In some embodiments, an individual is selected for treatment (e.g., using any of the methods of the present disclosure) if a sample (e.g., biopsy sample) obtained from the individual has at least one, at least two, at least three, at least four, or at least five high-power fields (HPFs) that each have a mast cell count of 10 or more mast cells per HPF (for a sample from the esophageal mucosa) and if a sample (e.g., biopsy sample) obtained from the individual does not have increased eosinophils, e.g., if a sample from the esophageal mucosa does not have at least one, at least two, at least three, at least four, or at least five HPFs that each have an eosinophil count of 10 or more eosinophils per HPF. For example, the eosinophil sample may have 1, 2, 3, 4, or 5 HPFs, or all HPFs tested, may fail to meet clinical criteria for eosinophilic disease.

In some embodiments, a sample from the esophageal mucosa has one or more HPFs that each have an eosinophil count of less than 15 eosinophils per HPF. In some embodiments, a sample from the esophageal mucosa does not have at least one, at least two, at least three, four, or five HPFs that each have an eosinophil count of 15 or more eosinophils per HPF. In some embodiments, an individual is selected for treatment (e.g., using any of the methods of the present disclosure) if a sample (e.g., biopsy sample) obtained from the individual has at least one, at least two, at least three, at least four, or at least five high-power fields (HPFs) that each have a mast cell count of 15 or more mast cells per HPF (for a sample from the esophageal mucosa) and if a sample (e.g., biopsy sample) obtained from the individual does not have increased eosinophils, e.g., if a sample from the esophageal mucosa does not have at least one, at least two, at least three, at least four, or at least five HPFs that each have an eosinophil count of 15 or more eosinophils per HPF. For example, the eosinophil sample may have 1, 2, 3, 4, or 5 HPFs, or all HPFs tested, may fail to meet clinical criteria for eosinophilic disease.

In some embodiments, multiple HPFs (e.g., 2, 3, 4, or 5 HPFs as described herein) can be obtained from a single biopsy (see Caldwell, J. M. et al. (2014) J. Allergy Clin. Immunol. 134:1114-1124), or in some cases from multiple biopsies. HPFs of the present disclosure may be obtained from 1, 2, 3, 4, or 5 samples (e.g., individual biopsies). In other words, by way of example, 5 HPFs may be from a total of 2 samples (e.g., 3 HPFs from one sample and 2 from the other, rather than requiring 5 HPFs from each of the two samples).

In some embodiments, a sample used for counting mast cells and/or eosinophils is obtained from a gastric or duodenal biopsy. In some embodiments, a sample used for counting mast cells and/or eosinophils is obtained from an esophageal biopsy. In some embodiments, a sample used for counting mast cells and/or eosinophils is obtained from an esophago-gastro-duodenoscopy (EGD) with biopsy. In some embodiments, multiple samples may be obtained from a single biopsy. For example, an esophageal biopsy may contain 4-6 samples representing different parts of the esophagus (e.g., 2 samples from the proximal esophagus, 2 samples from the middle esophagus, and 2 samples from the distal esophagus).

In some embodiments, the number of mast cells is compared with a mast cell reference. In some embodiments, the number of eosinophils is compared with an eosinophil reference. In some embodiments, a mast cell or eosinophil reference refers to a sample obtained from an individual that does not have esophagitis, gastritis, colitis, or gastroenteritis. In some embodiments, a mast cell or eosinophil reference refers to a numerical threshold value useful for diagnosing mast cell or eosinophilic esophagitis, gastritis, colitis, or gastroenteritis (e.g., a number of mast cells or eosinophils, optionally per HPF, used as a threshold in diagnosis, as described above). In some embodiments, a mast cell or eosinophil reference refers to an average value of mast cells or eosinophils obtained from multiple samples. As noted herein, a reference value and/or baseline value can be obtained from one individual, from two different individuals or from a group of individuals (e.g., a group of two, three, four, five or more individuals).

In some embodiments, number of mast cells in a sample is detected by hematoxylin and eosin (H&E) staining. In some embodiments, number of mast cells in a sample is detected by immunohistochemical (IHC) staining for a mast cell marker, including without limitation tryptase, CD117 (c-kit), or IgE receptor. In some embodiments, mast cell activation is detected (e.g., activated mast cells are detected) by IHC staining for tryptase and quantifying mast cell degranulation. In some embodiments, mast cell activation is detected (e.g., activated mast cells are detected) by staining for markers of mast cell activation (including without limitation CD63 and/or CD107a), e.g., by flow cytometry or IHC.

While detection of mast cells and eosinophils is described above using IHC and tissue samples, mast cell burden and/or activation can also be assayed by measuring biomarkers in serum, blood, or urine. In some embodiments, an individual that tests for abnormally high levels in one or more of these assays, and optionally does not test for high levels of eosinophils, may be treated using the methods of the present disclosure. In some embodiments, number and/or activity of mast cells in an individual are assayed by serum tryptase or beta tryptase. In some embodiments, number and/or activity of mast cells in an individual are assayed by blood level(s) of tryptase, histamine, leukotriene e4, prostaglandin D2, and/or heparin. In some embodiments, number and/or activity of mast cells in an individual are assayed by urine level(s) of N-methyl-histamine, prostaglandin F2 alpha, and/or prostaglandin D2.

B. Response to Treatment

In some embodiments, administering to an individual as described herein (e.g., an individual having mast cell gastritis, mast cell esophagitis, mast cell colitis, mast cell enteritis, mast cell duodenitis, and/or mast cell gastroenteritis) an effective amount of a composition of the present disclosure or antibody described herein that binds to human Siglec-8 (e.g., an anti-Siglec-8 antibody) reduces one or more (e.g., one or more, two or more, three or more, four or more, etc.) symptoms in the individual, as compared to a baseline level before administration of the antibody.

Response to treatment in individuals with mast cell gastritis, mast cell esophagitis, mast cell colitis, mast cell enteritis, mast cell duodenitis, and/or mast cell gastroenteritis can be assessed by various methods. For example, number, activity, and/or location of mast cells in a sample may be altered after treatment. In some embodiments, number of mast cells in a sample obtained from the individual after treatment is reduced, as compared with number of mast cells in a sample obtained prior to treatment. In some embodiments, activity of mast cells in the individual or in a sample obtained from the individual after treatment is reduced, as compared with activity of mast cells in the individual or in a sample obtained prior to treatment. In some embodiments, one or both of a number or activity of mast cells in a sample obtained from the gastric, duodenal, jejunal, ileal, or colonic mucosa of the individual are reduced after administration of a composition of the present disclosure as compared to a baseline level before administration of the composition. In some embodiments, one or more of a number, activity, or location of mast cells in a sample obtained from the esophageal mucosa of the individual is reduced after administration of a composition of the present disclosure as compared to a baseline level before administration of the composition.

In some embodiments, response to treatment with a composition or anti-Siglec-8 antibody of the present disclosure is assessed by expression level of one or more genes or polypeptides in a sample (e.g., a serum sample) obtained from the individual. For example, in some embodiments, serum tryptase; beta tryptase: blood level(s) of tryptase, histamine, leukotriene e4, prostaglandin D2, and/or heparin; and/or urine level(s) of N-methyl-histamine, prostaglandin F2 alpha, and/or prostaglandin D2 from the individual is/arc reduced, e.g., as compared to a baseline level obtained from the individual before administration of the composition or antibody, or as compared to a suitable reference value.

In some embodiments, one or more symptom(s) in the individual are reduced after administration of the composition as compared to a baseline level before administration of the composition. Symptoms for gastritis and gastroenteritis include, without limitation, abdominal pain, nausea, vomiting, loss of appetite, abdominal cramping, fullness before finishing a meal, bloating, diarrhea, and liquid or watery stools. Symptoms for esophagitis include, without limitation, heartburn, nausea, dysphagia/difficulty swallowing, vomiting, abdominal pain, cough, food impaction, early satiety, loss of appetite, chest pain, feeding intolerance or refusal, and gastroesophageal reflux. In some embodiments, one or more symptom(s) are monitored using a patient-reported outcome (PRO) questionnaire, which may be completed, e.g., daily. In some embodiments, one or more symptom(s) are monitored by adding or averaging scores from a daily patient-reported outcome (PRO) questionnaire obtained over a period of time, e.g., one week, two weeks, three weeks, four weeks/1 month, etc. In some embodiments, one or more symptom(s) in the individual are reduced by at least 50%, at least 55%, at least 60%, or at least 65% after administration of the composition as compared to a baseline level before administration of the composition, e.g., as measured by PRO questionnaire scores. For example, in some embodiments, one or more symptom(s) in the individual are reduced by at least 50%, at least 55%, at least 60%, or at least 65% after administration of the composition as compared to a baseline level before administration of the composition, e.g., as measured by mean or median PRO questionnaire scores over one week, two weeks, three weeks, or four weeks/I month.

In some embodiments, administration of a composition or anti-Siglec-8 antibody of the present disclosure results in a sustained response to treatment. In some embodiments, administration of a composition or antibody of the present disclosure results in a complete response to treatment (e.g., after cessation of treatment, or after a single dose of the antibody or composition).

The terms “baseline” or “baseline value” used interchangeably herein can refer to a measurement or characterization of a symptom before the administration of the therapy (e.g., an anti-Siglec-8 antibody) or at the beginning of administration of the therapy. The baseline value can be compared to a reference value in order to determine the reduction or improvement of a symptom of mast cell gastritis, mast cell esophagitis, mast cell colitis, mast cell enteritis, mast cell duodenitis, and/or mast cell gastroenteritis contemplated herein. A reference value and/or baseline value can be obtained from one individual, from two different individuals or from a group of individuals (e.g., a group of two, three, four, five or more individuals).

The terms “reference” or “reference value” used interchangeably herein can refer to a measurement or characterization of a value or symptom in an individual without mast cell gastritis or mast cell gastroenteritis (or in a group of such individuals). A “reference value” can be an absolute value; a relative value; a value that has an upper and/or lower limit, a range of values; an average value; a median value; a mean value; or a value as compared to a baseline value. Similarly, a “baseline value” can be an absolute value: a relative value; a value that has an upper and/or lower limit; a range of values; an average value; a median value; a mean value; or a value as compared to a reference value. A reference value can be obtained from one individual, from two different individuals or from a group of individuals (e.g., a group of two, three, four, five or more individuals). In some embodiments, a reference value refers to a standard or benchmark value in the field. In some embodiments, a reference value refers to a value calculated de novo from one or more individuals (e.g., without mast cell gastritis, mast cell esophagitis, mast cell colitis, or mast cell gastroenteritis).

C. Administration

In some embodiments, prior to administration of a composition or anti-Siglec-8 antibody of the present disclosure, the individual has failed or is not adequately controlled by one or more standard-of-care treatments for esophagitis, gastritis, and/or gastroenteritis. Exemplary standard-of-care treatments for esophagitis, gastritis, or gastroenteritis include, without limitation, proton pump inhibitor (PPI) treatment, corticosteroid treatment, and dietary treatment.

For the prevention or treatment of disease, the appropriate dosage of an active agent, will depend on the type of disease to be treated, as defined above, the severity and course of the disease, whether the agent is administered for preventive or therapeutic purposes, previous therapy, the individual's clinical history and response to the agent, and the discretion of the attending physician. The agent is suitably administered to the individual at one time or over a series of treatments. In some embodiments, an interval between administrations of an anti-Siglec-8 antibody (e.g., an antibody that binds to human Siglec-8) described herein is about one month or longer. In some embodiments, the interval between administrations is about 1 month, about two months, about three months, about four months, about five months, about six months or longer. As used herein, an interval between administrations refers to the time period between one administration of the antibody and the next administration of the antibody. As used herein, an interval of about one month includes four weeks. Accordingly, in some embodiments, the interval between administrations is about four weeks, about five weeks, about six weeks, about seven weeks, about eight weeks, about nine weeks, about ten weeks, about eleven weeks, about twelve weeks, about sixteen weeks, about twenty weeks, about twenty four weeks, or longer. In some embodiments, the treatment includes multiple administrations of the antibody, wherein the interval between administrations may vary. For example, the interval between the first administration and the second administration is about one month, and the intervals between the subsequent administrations are about three months. In some embodiments, the interval between the first administration and the second administration is about one month, the interval between the second administration and the third administration is about two months, and the intervals between the subsequent administrations are about three months. In some embodiments, an anti-Siglec-8 antibody described herein (e.g., an antibody that binds to human Siglec-8) is administered at a flat dose. In some embodiments, an anti-Siglec-8 antibody described herein (e.g., an antibody that binds to human Siglec-8) is administered to an individual at a dosage from about 0.1 mg to about 1800 mg per dose. In some embodiments, the anti-Siglec-8 antibody (e.g., an antibody that binds to human Siglec-8) is administered to an individual at a dosage of about any of 0.1 mg, 0.5 mg, 1 mg, 5 mg, 10 mg, 20 mg, 30 mg, 40 mg, 50 mg, 60 mg, 70 mg, 80 mg, 90 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1100 mg, 1200 mg, 1300 mg, 1400 mg, 1500 mg, 1600 mg, 1700 mg, and 1800 mg per dose. In some embodiments, an anti-Siglec-8 antibody described herein (e.g., an antibody that binds to human Siglec-8) is administered to an individual at a dosage from about 150 mg to about 450 mg per dose. In some embodiments, the anti-Siglec-8 antibody (e.g., an antibody that binds to human Siglec-8) is administered to an individual at a dosage of about any of 150 mg, 200 mg, 250 mg, 300 mg, 350 mg, 400 mg, and 450 mg per dose. In some embodiments, an anti-Siglec-8 antibody described herein (e.g., an antibody that binds to human Siglec-8) is administered to an individual at a dosage from about 0.1 mg/kg to about 20 mg/kg per dose. In some embodiments, an anti-Siglec-8 antibody described herein (e.g., an antibody that binds to human Siglec-8) is administered to an individual at a dosage from about 0.01 mg/kg to about 10 mg/kg per dose. In some embodiments, an anti-Siglec-8 antibody described herein (e.g., an antibody that binds to human Siglec-8) is administered to an individual at a dosage from about 0.1 mg/kg to about 10 mg/kg, about 1.0 mg/kg to about 10 mg/kg, or about 0.3 mg/kg to about 1.0 mg/kg. In some embodiments, an anti-Siglec-8 antibody described herein is administered to an individual at a dosage of about any of 0.1 mg/kg, 0.3 mg/kg, 0.4 mg/kg, 0.5 mg/kg, 0.6 mg/kg, 0.7 mg/kg, 0.8 mg/kg, 0.9 mg/kg, 1.0 mg/kg, 1.5 mg/kg, 2.0 mg/kg, 2.5 mg/kg, 3.0 mg/kg, 3.5 mg/kg, 4.0 mg/kg, 4.5 mg/kg, 5.0 mg/kg, 5.5 mg/kg, 6.0 mg/kg, 6.5 mg/kg, 7.0 mg/kg, 7.5 mg/kg, 8.0 mg/kg, 8.5 mg/kg, 9.0 mg/kg, 9.5 mg/kg, or 10.0 mg/kg. Any of the dosing frequency described above may be used. Any dosing frequency described above may be used in the methods or uses of the compositions described herein. Efficacy of treatment with an antibody described herein (e.g., an antibody that binds to human Siglec-8) can be assessed using any of the methodologies or assays described herein at intervals ranging between every week and every three months. In some embodiments, efficacy of treatment (e.g., reduction or improvement of one or more symptoms) is assessed about every one month, about every two months, about every three months, about every four months, about every five months, about every six months or longer after administration of an antibody that binds to human Siglec-8. In some embodiments, efficacy of treatment (e.g., reduction or improvement of one or more symptoms) is assessed about every one week, about every two weeks, about every three weeks, about every four weeks, about every five weeks, about every six weeks, about every seven weeks, about every eight weeks, about every nine weeks, about every ten weeks, about every eleven weeks, about every twelve weeks, about every sixteen weeks, about every twenty weeks, about every twenty four weeks, or longer.

In some embodiments, an anti-Siglec-8 antibody described herein (e.g., an antibody that binds to human Siglec-8) is administered to an individual (e.g., by intravenous infusion) at one or more doses comprising between about 0.1 mg/kg and about 4.0 mg/kg of the antibody. In some embodiments, the antibody is administered to an individual by intravenous infusion at one or more doses comprising between about 0.3 mg/kg and about 3.0 mg/kg of the antibody, e.g., at about 0.3 mg/kg antibody, about 0.5 mg/kg antibody, about 1.0 mg/kg antibody, about 1.5 mg/kg antibody, about 2.0 mg/kg antibody, about 2.5 mg/kg antibody, or about 3.0 mg/kg antibody. In some embodiments, the antibody is administered to the individual (e.g., by intravenous infusion) in two or more doses (e.g., comprising between about 0.3 mg/kg and about 3.0 mg/kg of the antibody) at an interval of about 28 days. In some embodiments, the antibody is administered to the individual (e.g., by intravenous infusion) monthly in two or more doses (e.g., comprising between about 0.3 mg/kg and about 3.0 mg/kg of the antibody). In some embodiments, the antibody is administered to the individual (e.g., by intravenous infusion) in two or more doses (e.g., comprising between about 0.3 mg/kg and about 3.0 mg/kg of the antibody) at an interval of about 4 weeks. In some embodiments, the antibody is administered to the individual (e.g., by intravenous infusion) according to the following schedule: Day 1. Day 29, Day 57. Day 85, Day 113, and Day 141. In some embodiments, the antibody is administered to the individual by intravenous infusion at a first dose comprising about 0.3 mg/kg of the antibody, a second dose comprising about 1.0 mg/kg of the antibody, a third dose comprising about 1.0 mg/kg of the antibody, a fourth dose comprising about 1.0 mg/kg to about 3.0 mg/kg of the antibody, a fifth dose comprising about 1.0 mg/kg to about 3.0 mg/kg of the antibody, and a sixth dose comprising about 1.0 mg/kg to about 3.0 mg/kg of the antibody. In some embodiments, the antibody is administered to the individual by intravenous infusion at a first dose comprising about 0.3 mg/kg of the antibody, a second dose comprising about 1.0 mg/kg of the antibody, a third dose comprising about 1.0 mg/kg of the antibody, a fourth dose comprising about 1.0 mg/kg or about 3.0 mg/kg of the antibody, a fifth dose comprising about 1.0 mg/kg or about 3.0 mg/kg of the antibody, and a sixth dose comprising about 1.0 mg/kg or about 3.0 mg/kg of the antibody. In some embodiments, the antibody is administered to the individual by intravenous infusion at a first dose comprising about 0.3 mg/kg of the antibody, a second dose comprising about 1.0 mg/kg of the antibody, a third dose comprising about 1.0 mg/kg of the antibody, a fourth dose comprising about 1.0 mg/kg of the antibody, a fifth dose comprising about 1.0 mg/kg of the antibody, and a sixth dose comprising about 1.0 mg/kg of the antibody. In some embodiments, the antibody is administered to the individual by intravenous infusion according to the following schedule: about 0.3 mg/kg of the antibody on Day 1, about 1.0 mg/kg of the antibody on Day 29, about 1.0 mg/kg of the antibody on Day 57, about 1.0 mg/kg or about 3.0 mg/kg of the antibody on Day 85, about 1.0 mg/kg or about 3.0 mg/kg of the antibody on Day 113, and about 1.0 mg/kg or about 3.0 mg/kg of the antibody on Day 141.

Antibodies described herein that bind to human Siglec-8 can be used either alone or in combination with other agents in the methods described herein. For instance, an antibody that binds to a human Siglec-8 may be co-administered with one or more (e.g., one or more, two or more, three or more, four or more, etc.) additional therapeutic agents for treating and/or preventing gastritis, esophagitis, and/or gastroenteritis. Additional therapeutic agents for treating and/or preventing gastritis, esophagitis, and/or gastroenteritis include, without limitation, PPIs, systemic corticosteroids, topical corticosteroids, antihistamines, mast cell stabilizers, H-2 blockers, anti-IgE antibodies, calcineurin inhibitors, immunomodulatory agents, and immunosuppressive agents (e.g., azathioprine, 6-MP, MMF, and mTOR inhibitors).

Such combination therapies noted above encompass combined administration (where two or more therapeutic agents are included in the same or separate formulations), and separate administration, in which case, administration of the antibody of the present disclosure can occur prior to, simultaneously, and/or following, administration of the one or more additional therapeutic agents. In some embodiments, administration of an anti-Siglec-8 antibody described herein and administration of one or more additional therapeutic agents occur within about one month, about two months, about three months, about four months, about five months or about six months of each other. In some embodiments, administration of an anti-Siglec-8 antibody described herein and administration of one or more additional therapeutic agents occur within about one week, about two weeks or about three weeks of each other. In some embodiments, administration of an anti-Siglec-8 antibody described herein and administration of one or more additional therapeutic agents occur within about one day, about two days, about three days, about four days, about five days, or about six days of each other.

Anti-Siglec8 antibodies and/or one or more additional therapeutic agents may be administered via any suitable route of administration known in the art, including, without limitation, by oral administration, sublingual administration, buccal administration, topical administration, rectal administration, via inhalation, transdermal administration, subcutaneous injection, intradermal injection, intravenous (IV) injection, intra-arterial injection, intramuscular injection, intracardiac injection, intraosseous injection, intraperitoneal injection, transmucosal administration, vaginal administration, intravitreal administration, intra-articular administration, peri-articular administration, local administration, epicutaneous administration, or any combinations thereof.

D. Antibodies

Certain aspects of the present disclosure provide isolated antibodies that bind to a human Siglec-8 (e.g., an agonist antibody that binds to human Siglec-8). In some embodiments, an anti-Siglec-8 antibody described herein has one or more of the following characteristics: (1) binds a human Siglec-8; (2) binds to an extracellular domain of a human Siglec-8; (3) binds a human Siglec-8 with a higher affinity than mouse antibody 2E2 and/or mouse antibody 2C4; (4) binds a human Siglec-8 with a higher avidity than mouse antibody 2E2 and/or mouse antibody 2C4; (5) has a Tₘ of about 70° C.-72° C. or higher in a thermal shift assay; (6) has a reduced degree of fucosylation or is non-fucosylated; (7) binds a human Siglec-8 expressed on eosinophils and induces apoptosis of eosinophils; (8) binds a human Siglec-8 expressed on mast cells and depletes or reduces the number of mast cells; (9) binds a human Siglec-8 expressed on mast cells and inhibits FcεRI-dependent activities of mast cells (e.g., histamine release, PGD2 release, Ca²⁺ flux, and/or β-hexosaminidase release, etc.); (10) has been engineered to improve ADCC activity; (11) binds a human Siglec-8 expressed on mast cells and kills mast cells by ADCC activity (in vitro, and/or in vivo); (12) binds to Siglec-8 of a human and a non-human primate; (13) binds to Domain 1, Domain 2, and/or Domain 3 of human Siglec-8, or binds a Siglec-8 polypeptide comprising Domain 1, Domain 2, and/or Domain 3 of human Siglec-8 (e.g., fusion proteins described herein); and (14) depletes activated eosinophils with an EC₅₀ less than the EC₅₀ of mouse antibody 2E2 or 2C4. Any of the antibodies described in U.S. Pat. No. 9,546,215 and/or WO2015089117 may find use in the methods, compositions, and kits provided herein.

In one aspect, the present disclosure provides antibodies that bind to a human Siglec-8. In some embodiments, the human Siglec-8 comprises an amino acid sequence of SEQ ID NO:72. In some embodiments, the human Siglec-8 comprises an amino acid sequence of SEQ ID NO:73. In some embodiments, an antibody described herein binds to a human Siglec-8 expressed on mast cells and depletes or reduces the number of mast cells. In some embodiments, an antibody described herein binds to a human Siglec-8 expressed on mast cells and inhibits mast cell-mediated activity.

In one aspect, the invention provides antibodies that bind to a human Siglec-8. In some embodiments, the human Siglec-8 comprises an amino acid sequence of SEQ ID NO:72. In some embodiments, the human Siglec-8 comprises an amino acid sequence of SEQ ID NO:73. In some embodiments, the antibody described herein binds to an epitope in Domain 1 of human Siglec-8, wherein Domain 1 comprises the amino acid sequence of SEQ ID NO: 112. In some embodiments, the antibody described herein binds to an epitope in Domain 2 of human Siglec-8, wherein Domain 2 comprises the amino acid sequence of SEQ ID NO: 113. In some embodiments, the antibody described herein binds to an epitope in Domain 3 of human Siglec-8, wherein Domain 3 comprises the amino acid sequence of SEQ ID NO: 114. In some embodiments, the antibody described herein binds to a fusion protein comprising the amino acid of SEQ ID NO:116 but not to a fusion protein comprising the amino acid of SEQ ID NO:115. In some embodiments, the antibody described herein binds to a fusion protein comprising the amino acid of SEQ ID NO:117 but not to a fusion protein comprising the amino acid of SEQ ID NO:115. In some embodiments, the antibody described herein binds to a fusion protein comprising the amino acid of SEQ ID NO:117 but not to a fusion protein comprising the amino acid of SEQ ID NO: 116. In some embodiments, the antibody described herein binds to a linear epitope in the extracellular domain of human Siglec-8. In some embodiments, the antibody described herein binds to a conformational epitope in the extracellular domain of human Siglec-8. In some embodiments, an antibody described herein binds to a human Siglec-8 expressed on eosinophils and induces apoptosis of eosinophils. In some embodiments, an antibody described herein binds to a human Siglec-8 expressed on mast cells and depletes mast cells. In some embodiments, an antibody described herein binds to a human Siglec-8 expressed on mast cells and inhibits mast cell-mediated activity. In some embodiments, an antibody described herein binds to a human Siglec-8 expressed on mast cells and kills mast cells by ADCC activity. In some embodiments, an antibody described herein depletes mast cells and inhibits mast cell activation. In some embodiments, an antibody herein depletes activated eosinophils and inhibits mast cell activation. In some embodiments, an antibody herein (e.g., a non-fucosylated anti-Siglec-8 antibody) depletes blood eosinophils and inhibits mast cell activation. In some embodiments, an antibody herein (e.g., a non-fucosylated anti-Siglec-8 antibody) depletes eosinophils from the peripheral blood and inhibits mast cell activation.

Provided herein is an isolated anti-Siglec-8 antibody that binds to human Siglec-8 and non-human primate Siglec-8. Identification of antibodies with primate cross-reactivity would be useful for preclinical testing of anti-Siglec-8 antibodies in non-human primates. In one aspect, the invention provides antibodies that bind to a non-human primate Siglec-8. In one aspect, the invention provides antibodies that bind to a human Siglec-8 and a non-human primate Siglec-8. In some embodiments, the non-human primate Siglec-8 comprises an amino acid sequence of SEQ ID NO:118 or a portion thereof. In some embodiments, the non-human primate Siglec-8 comprises an amino acid sequence of SEQ ID NO:119 or a portion thereof. In some embodiments, the non-human primate is a baboon (e.g., Papio Anubis). In some embodiments, the antibody that binds to a human Siglec-8 and a non-human primate Siglec-8, binds to an epitope in Domain 1 of human Siglec-8. In a further embodiment, Domain 1 of human Siglec-8 comprises the amino acid sequence of SEQ ID NO:112. In some embodiments, the antibody that binds to a human Siglec-8 and a non-human primate Siglec-8, binds to an epitope in Domain 3 of human Siglec-8. In a further embodiment. Domain 3 of human Siglec-8 comprises the amino acid sequence of SEQ ID NO:114. In some embodiments, the antibody that binds to a human Siglec-8 and a non-human primate Siglec-8 is a humanized antibody, a chimeric antibody, or a human antibody. In some embodiments, the antibody that binds to a human Siglec-8 and a non-human primate Siglec-8 is a murine antibody. In some embodiments, the antibody that binds to a human Siglec-8 and a non-human primate Siglec-8 is a human IgG1 antibody.

In one aspect, an anti-Siglec-8 antibody described herein is a monoclonal antibody. In one aspect, an anti-Siglec-8 antibody described herein is an antibody fragment (including antigen-binding fragment), e.g., a Fab, Fab′-SH, Fv, scFv, or (Fab′) fragment. In one aspect, an anti-Siglec-8 antibody described herein comprises an antibody fragment (including antigen-binding fragment), e.g., a Fab, Fab′-SH, Fv, scFv, or (Fab′)₂ fragment. In one aspect, an anti-Siglec-8 antibody described herein is a chimeric, humanized, or human antibody. In one aspect, any of the anti-Siglec-8 antibodies described herein are purified.

In one aspect, anti-Siglec-8 antibodies that compete with murine 2E2 antibody and murine 2C4 antibody binding to Siglec-8 are provided. Anti-Siglec-8 antibodies that bind to the same epitope as murine 2E2 antibody and murine 2C4 antibody are also provided. Murine antibodies to Siglec-8, 2E2 and 2C4 antibody are described in U.S. Pat. Nos. 8,207,305; 8,197,811, 7,871,612, and 7,557,191.

In one aspect, anti-Siglec-8 antibodies that compete with any anti-Siglec-8 antibody described herein (e.g., HEKA, HEKF, 1C3, 1H10, 4F11, 2C4, 2E2) for binding to Siglec-8 are provided. Anti-Siglec-8 antibodies that bind to the same epitope as any anti-Siglec-8 antibody described herein (e.g., HEKA, HEKF, 1C3, 1H10, 4F11, 2C4, 2E2) are also provided.

In one aspect of the present disclosure, polynucleotides encoding anti-Siglec-8 antibodies are provided. In certain embodiments, vectors comprising polynucleotides encoding anti-Siglec-8 antibodies are provided. In certain embodiments, host cells comprising such vectors are provided. In another aspect of the present disclosure, compositions comprising anti-Siglec-8 antibodies or polynucleotides encoding anti-Siglec-8 antibodies are provided. In certain embodiments, a composition of the present disclosure is a pharmaceutical formulation for the treatment of mast cell gastritis, mast cell esophagitis, mast cell colitis, mast cell enteritis, mast cell duodenitis, and/or mast cell gastroenteritis. In certain embodiments, a composition of the present disclosure is a pharmaceutical formulation for the prevention of mast cell gastritis, mast cell esophagitis, mast cell colitis, mast cell enteritis, mast cell duodenitis, and/or mast cell gastroenteritis.

In one aspect, provided herein is an anti-Siglec-8 antibody comprising 1, 2, 3, 4, 5, or 6 of the HVR sequences of the murine antibody 2C4. In one aspect, provided herein is an anti-Siglec-8 antibody comprising 1, 2, 3, 4, 5, or 6 of the HVR sequences of the murine antibody 2E2. In some embodiments, the HVR is a Kabat CDR or a Chothia CDR.

In one aspect, provided herein is an anti-Siglec-8 antibody comprising 1, 2, 3, 4, 5, or 6 of the HVR sequences of the murine antibody 1C3. In one aspect, provided herein is an anti-Siglec-8 antibody comprising 1, 2, 3, 4, 5, or 6 of the HVR sequences of the murine antibody 4F11. In one aspect, provided herein is an anti-Siglec-8 antibody comprising 1, 2, 3, 4, 5, or 6 of the HVR sequences of the murine antibody 1H10. In some embodiments, the HVR is a Kabat CDR or a Chothia CDR.

In some embodiments, the antibody described herein binds to an epitope in Domain 1 of human Siglec-8, wherein Domain 1 comprises the amino acid sequence of SEQ ID NO:112. In some embodiments, the antibody described herein binds to an epitope in Domain 2 of human Siglec-8, wherein Domain 2 comprises the amino acid sequence of SEQ ID NO:113. In some embodiments, the antibody described herein binds to an epitope in Domain 3 of human Siglec-8, wherein Domain 3 comprises the amino acid sequence of SEQ ID NO:114.

In some embodiments, the antibody described herein binds to a fusion protein comprising the amino acid of SEQ ID NO:116 but not to a fusion protein comprising the amino acid of SEQ ID NO: 115. In some embodiments, the antibody described herein binds to a fusion protein comprising the amino acid of SEQ ID NO:117 but not to a fusion protein comprising the amino acid of SEQ ID NO:115. In some embodiments, the antibody described herein binds to a fusion protein comprising the amino acid of SEQ ID NO:117 but not to a fusion protein comprising the amino acid of SEQ ID NO:116.

In another aspect, provided herein is an anti-Siglec-8 antibody comprising a heavy chain variable region and a light chain variable region, wherein the heavy chain variable region comprises (i) HVR-H1 comprising the amino acid sequence of SEQ ID NO:88, (ii) HVR-H2 comprising the amino acid sequence of SEQ ID NO:91, and (iii) HVR-H3 comprising the amino acid sequence of SEQ ID NO:94; and/or a light chain variable region comprising (i) HVR-L1 comprising the amino acid sequence of SEQ ID NO:97, (ii) HVR-L2 comprising the amino acid sequence of SEQ ID NO:100, and (iii) HVR-L3 comprising the amino acid sequence of SEQ ID NO:103. In some embodiments, the antibody described herein binds to an epitope in Domain 2 of human Siglec-8, wherein Domain 2 comprises the amino acid sequence of SEQ ID NO: 113.

In another aspect, provided herein is an anti-Siglec-8 antibody comprising a heavy chain variable region and a light chain variable region, wherein the heavy chain variable region comprises (i) HVR-H1 comprising the amino acid sequence of SEQ ID NO:89, (ii) HVR-H2 comprising the amino acid sequence of SEQ ID NO:92, and (iii) HVR-H3 comprising the amino acid sequence of SEQ ID NO:95; and/or a light chain variable region comprising (i) HVR-L1 comprising the amino acid sequence of SEQ ID NO:98, (ii) HVR-L2 comprising the amino acid sequence of SEQ ID NO:101, and (iii) HVR-L3 comprising the amino acid sequence of SEQ ID NO:104. In some embodiments, the antibody described herein binds to an epitope in Domain 3 of human Siglec-8, wherein Domain 3 comprises the amino acid sequence of SEQ ID NO: 114. In some embodiments, the antibody described herein binds to human Siglec-8 and non-human primate Siglec-8.

In another aspect, provided herein is an anti-Siglec-8 antibody comprising a heavy chain variable region and a light chain variable region, wherein the heavy chain variable region comprises (i) HVR-H1 comprising the amino acid sequence of SEQ ID NO:90, (ii) HVR-H2 comprising the amino acid sequence of SEQ ID NO:93, and (iii) HVR-H3 comprising the amino acid sequence of SEQ ID NO:96; and/or a light chain variable region comprising (i) HVR-L1 comprising the amino acid sequence of SEQ ID NO:99, (ii) HVR-L2 comprising the amino acid sequence of SEQ ID NO:102, and (iii) HVR-L3 comprising the amino acid sequence of SEQ ID NO:105. In some embodiments, the antibody described herein binds to an epitope in Domain 1 of human Siglec-8, wherein Domain 1 comprises the amino acid sequence of SEQ ID NO: 112. In some embodiments, the antibody described herein binds to human Siglec-8 and non-human primate Siglec-8.

In one aspect, provided herein is an anti-Siglec-8 antibody comprising a heavy chain variable region and a light chain variable region, wherein the heavy chain variable region comprises (i) HVR-H1 comprising the amino acid sequence of SEQ ID NO:61, (ii) HVR-H2 comprising the amino acid sequence of SEQ ID NO:62, and (iii) HVR-H3 comprising the amino acid sequence of SEQ ID NO:63; and/or wherein the light chain variable region comprises (i) HVR-L1 comprising the amino acid sequence of SEQ ID NO:64, (ii) HVR-L2 comprising the amino acid sequence of SEQ ID NO:65, and (iii) HVR-L3 comprising the amino acid sequence of SEQ ID NO:66.

In one aspect, provided herein is an anti-Siglec-8 antibody comprising a heavy chain variable region and a light chain variable region, wherein the heavy chain variable region comprises (i) HVR-H1 comprising the amino acid sequence of SEQ ID NO:61, (ii) HVR-H2 comprising the amino acid sequence of SEQ ID NO:62, and (iii) HVR-H3 comprising the amino acid sequence selected from SEQ ID NOs:67-70; and/or wherein the light chain variable region comprises (i) HVR-L1 comprising the amino acid sequence of SEQ ID NO:64, (ii) HVR-L2 comprising the amino acid sequence of SEQ ID NO:65, and (iii) HVR-L3 comprising the amino acid sequence of SEQ ID NO:66.

In one aspect, provided herein is an anti-Siglec-8 antibody comprising a heavy chain variable region and a light chain variable region, wherein the heavy chain variable region comprises (i) HVR-H1 comprising the amino acid sequence of SEQ ID NO:61, (ii) HVR-H2 comprising the amino acid sequence of SEQ ID NO:62, and (iii) HVR-H3 comprising the amino acid sequence of SEQ ID NO:63; and/or wherein the light chain variable region comprises (i) HVR-L1 comprising the amino acid sequence of SEQ ID NO:64, (ii) HVR-L2 comprising the amino acid sequence of SEQ ID NO:65, and (iii) HVR-L3 comprising the amino acid sequence of SEQ ID NO:71.

In another aspect, provided herein is an anti-Siglec-8 antibody comprising a heavy chain variable region and a light chain variable region, wherein the heavy chain variable region comprises (i) HVR-H1 comprising the amino acid sequence of SEQ ID NO:61, (ii) HVR-H2 comprising the amino acid sequence of SEQ ID NO:62, and (iii) HVR-H3 comprising the amino acid sequence selected from SEQ ID NOs:67-70; and/or wherein the light chain variable region comprises (i) HVR-L1 comprising the amino acid sequence of SEQ ID NO:64, (ii) HVR-L2 comprising the amino acid sequence of SEQ ID NO:65, and (iii) HVR-L3 comprising the amino acid sequence of SEQ ID NO:71.

In another aspect, provided herein is an anti-Siglec-8 antibody comprising a heavy chain variable region and a light chain variable region, wherein the heavy chain variable region comprises (i) HVR-H1 comprising the amino acid sequence of SEQ ID NO:88, (ii) HVR-H2 comprising the amino acid sequence of SEQ ID NO:91, and (iii) HVR-H3 comprising the amino acid sequence of SEQ ID NO:94; and/or a light chain variable region comprising (i) HVR-L1 comprising the amino acid sequence of SEQ ID NO:97, (ii) HVR-L2 comprising the amino acid sequence of SEQ ID NO:100, and (iii) HVR-L3 comprising the amino acid sequence of SEQ ID NO:103.

In another aspect, provided herein is an anti-Siglec-8 antibody comprising a heavy chain variable region and a light chain variable region, wherein the heavy chain variable region comprises (i) HVR-H1 comprising the amino acid sequence of SEQ ID NO:89. (ii) HVR-H2 comprising the amino acid sequence of SEQ ID NO:92, and (iii) HVR-H3 comprising the amino acid sequence of SEQ ID NO:95; and/or a light chain variable region comprising (i) HVR-L1 comprising the amino acid sequence of SEQ ID NO:98, (ii) HVR-L2 comprising the amino acid sequence of SEQ ID NO:101, and (iii) HVR-L3 comprising the amino acid sequence of SEQ ID NO:104.

In another aspect, provided herein is an anti-Siglec-8 antibody comprising a heavy chain variable region and a light chain variable region, wherein the heavy chain variable region comprises (i) HVR-H1 comprising the amino acid sequence of SEQ ID NO:90, (ii) HVR-H2 comprising the amino acid sequence of SEQ ID NO:93, and (iii) HVR-H3 comprising the amino acid sequence of SEQ ID NO:96; and/or a light chain variable region comprising (i) HVR-L1 comprising the amino acid sequence of SEQ ID NO:99, (ii) HVR-L2 comprising the amino acid sequence of SEQ ID NO:102, and (iii) HVR-L3 comprising the amino acid sequence of SEQ ID NO:105.

An anti-Siglec-8 antibody described herein may comprise any suitable framework variable domain sequence, provided that the antibody retains the ability to bind human Siglec-8. As used herein, heavy chain framework regions are designated “HC-FR1-FR4,” and light chain framework regions are designated “LC-FR1-FR4.” In some embodiments, the anti-Siglec-8 antibody comprises a heavy chain variable domain framework sequence of SEQ ID NO:26, 34, 38, and 45 (HC-FR1. HC-FR2, HC-FR3, and HC-FR4, respectively). In some embodiments, the anti-Siglec-8 antibody comprises a light chain variable domain framework sequence of SEQ ID NO:48, 51, 55, and 60 (LC-FR1. LC-FR2, LC-FR3, and LC-FR4, respectively). In some embodiments, the anti-Siglec-8 antibody comprises a light chain variable domain framework sequence of SEQ ID NO:48, 51, 58, and 60 (LC-FR1, LC-FR2, LC-FR3, and LC-FR4, respectively).

In one embodiment, an anti-Siglec-8 antibody comprises a heavy chain variable domain comprising a framework sequence and hypervariable regions, wherein the framework sequence comprises the HC-FR1-HC-FR4 sequences SEQ ID NOs:26-29 (HC-FR1), SEQ ID NOs:31-36 (HC-FR2), SEQ ID NOs:38-43 (HC-FR3), and SEQ ID NOs:45 or 46 (HC-FR4), respectively; the HVR-H1 comprises the amino acid sequence of SEQ ID NO:61; the HVR-H2 comprises the amino acid sequence of SEQ ID NO:62, and the HVR-H3 comprises an amino acid sequence of SEQ ID NO:63. In one embodiment, an anti-Siglec-8 antibody comprises a heavy chain variable domain comprising a framework sequence and hypervariable regions, wherein the framework sequence comprises the HC-FR1-HC-FR4 sequences SEQ ID NOs:26-29 (HC-FR1), SEQ ID NOs:31-36 (HC-FR2), SEQ ID NOs:38-43 (HC-FR3), and SEQ ID NOs:45 or 46 (HC-FR4), respectively; the HVR-H1 comprises the amino acid sequence of SEQ ID NO:61, the HVR-H2 comprises the amino acid sequence of SEQ ID NO:62; and the HVR-H3 comprises an amino acid sequence selected from SEQ ID NOs:67-70. In one embodiment, an anti-Siglec-8 antibody comprises a light chain variable domain comprising a framework sequence and hypervariable regions, wherein the framework sequence comprises the LC-FR1-LC-FR4 sequences SEQ ID NOs:48 or 49 (LC-FR1), SEQ ID NOs:51-53 (LC-FR2), SEQ ID NOs:55-58 (LC-FR3), and SEQ ID NO:60 (LC-FR4), respectively, the HVR-L1 comprises the amino acid sequence of SEQ ID NO:64; the HVR-L2 comprises the amino acid sequence of SEQ ID NO:65; and the HVR-L3 comprises an amino acid sequence of SEQ ID NO:66. In one embodiment, an anti-Siglec-8 antibody comprises a light chain variable domain comprising a framework sequence and hypervariable regions, wherein the framework sequence comprises the LC-FR1-LC-FR4 sequences SEQ ID NOs:48 or 49 (LC-FR1), SEQ ID NOs:51-53 (LC-FR2), SEQ ID NOs:55-58 (LC-FR3), and SEQ ID NO:60 (LC-FR4), respectively; the HVR-L1 comprises the amino acid sequence of SEQ ID NO:64; the HVR-L2 comprises the amino acid sequence of SEQ ID NO:65; and the HVR-L3 comprises an amino acid sequence of SEQ ID NO:71. In one embodiment of these antibodies, the heavy chain variable domain comprises an amino acid sequence selected from SEQ ID NOs:2-10 and the light chain variable domain comprises and amino acid sequence selected from SEQ ID NOs:16-22. In one embodiment of these antibodies, the heavy chain variable domain comprises an amino acid sequence selected from SEQ ID NOs:2-10 and the light chain variable domain comprises and amino acid sequence selected from SEQ ID NOs:23 or 24. In one embodiment of these antibodies, the heavy chain variable domain comprises an amino acid sequence selected from SEQ ID NOs: 11-14 and the light chain variable domain comprises and amino acid sequence selected from SEQ ID NOs:16-22. In one embodiment of these antibodies, the heavy chain variable domain comprises an amino acid sequence selected from SEQ ID NOs:11-14 and the light chain variable domain comprises and amino acid sequence selected from SEQ ID NOs:23 or 24. In one embodiment of these antibodies, the heavy chain variable domain comprises an amino acid sequence of SEQ ID NO:6 and the light chain variable domain comprises and amino acid sequence of SEQ ID NO:16. In one embodiment of these antibodies, the heavy chain variable domain comprises an amino acid sequence of SEQ ID NO:6 and the light chain variable domain comprises and amino acid sequence of SEQ ID NO:21.

In some embodiments, the heavy chain HVR sequences comprise the following:

a) HVR-H1 (IYGAH (SEQ ID NO: 61));
b) HVR-H2 (VIWAGGSTNYNSALMS (SEQ ID NO: 62));
and
c) HVR-H3 (DGSSPYYYSMEY (SEQ ID NO: 63);
DGSSPYYYGMEY (SEQ ID NO: 67);
DGSSPYYYSMDY (SEQ ID NO: 68);
DGSSPYYYSMEV (SEQ ID NO: 69);
or
DGSSPYYYGMDV (SEQ ID NO: 70)).

In some embodiments, the heavy chain HVR sequences comprise the following:

a) HVR-H1 (SYAMS (SEQ ID NO: 88);
DYYMY (SEQ ID NO: 89);
or
SSWMN SEQ ID NO: 90));
b) HVR-H2 (IISSGGSYTYYSDSVKG (SEQ ID NO: 91);
RIAPEDGDTEYAPKFQG (SEQ ID NO: 92);
or
QIYPGDDYTNYNGKFKG (SEQ ID NO: 93));
and
c) HVR-H3 (HETAQAAWFAY (SEQ ID NO: 94);
EGNYYGSSILDY (SEQ ID NO: 95);
or
LGPYGPFAD (SEQ ID NO: 96)).

In some embodiments, the heavy chain FR sequences comprise the following:

a) HC-FR1
(EVQLVESGGGLVQPGGSLRLSCAASGFSLT (SEQ ID NO: 26);
EVQLVESGGGLVQPGGSLRLSCAVSGFSLT (SEQ ID NO: 27);
QVQLQESGPGLVKPSETLSLTCTVSGGSIS (SEQ ID NO: 28);
or
QVQLQESGPGLVKPSETLSLTCTVSGFSLT (SEQ ID NO: 29));
b) HC-FR2
(WVRQAPGKGLEWVS (SEQ ID NO: 31);
WVRQAPGKGLEWLG (SEQ ID NO: 32);
WVRQAPGKGLEWLS (SEQ ID NO: 33);
WVRQAPGKGLEWVG (SEQ ID NO: 34);
WIRQPPGKGLEWIG (SEQ ID NO: 35);
or
WVRQPPGKGLEWLG (SEQ ID NO: 36));
c) HC-FR3
(RFTISKDNSKNTVYLQMNSLRAEDTAVYYCAR (SEQ ID NO: 38);
RLSISKDNSKNTVYLQMNSLRAEDTAVYYCAR (SEQ ID NO: 39);
RLTISKDNSKNTVYLQMNSLRAEDTAVYYCAR (SEQ ID NO: 40);
RFSISKDNSKNTVYLQMNSLRAEDTAVYYCAR (SEQ ID NO: 41);
RVTISVDTSKNQFSLKLSSVTAADTAVYYCAR (SEQ ID NO: 42);
or
RLSISKDNSKNQVSLKLSSVTAADTAVYYCAR (SEQ ID NO: 43));
and
d) HC-FR4 (WGQGVTTVTVSS (SEQ ID NO: 45);
or
WGQGTLVTVSS (SEQ ID NO: 46)).

In some embodiments, the light chain HVR sequences comprise the following:

a) HVR-L1 (SATSSVSYMH (SEQ ID NO: 64));
b) HVR-L2 (STSNLAS (SEQ ID NO: 65));
and
c) HVR-L3 (QQRSSYPFT (SEQ ID NO: 66);
or
QQRSSYPYT (SEQ ID NO: 71)).

In some embodiments, the light chain HVR sequences comprise the following:

a) HVR-L1 (SASSSVSYMH (SEQ ID NO: 97);
RASQDITNYLN (SEQ ID NO: 98);
or
SASSSVSYMY (SEQ ID NO: 99));
b) HVR-L2 (DTSKLAY (SEQ ID NO: 100);
FTSRLHS (SEQ ID NO: 101);
or
DTSSLAS (SEQ ID NO: 102));
and
c) HVR-L3 (QQWSSNPPT (SEQ ID NO: 103);
QQGNTLPWT (SEQ ID NO: 104);
or
QQWNSDPYT (SEQ ID NO: 105)).

In some embodiments, the antibody comprises:
a heavy chain variable region comprising (i) HVR-H1 comprising the amino acid sequence of SEQ ID NO:88, (ii) HVR-H2 comprising the amino acid sequence of SEQ ID NO:91, and (iii) HVR-H3 comprising the amino acid sequence of SEQ ID NO:94; and/or a light chain variable region comprising (i) HVR-L1 comprising the amino acid sequence of SEQ ID NO:97, (ii) HVR-L2 comprising the amino acid sequence of SEQ ID NO:100, and (iii) HVR-L3 comprising the amino acid sequence of SEQ ID NO:103;
a heavy chain variable region comprising (i) HVR-H1 comprising the amino acid sequence of SEQ ID NO:89. (ii) HVR-H2 comprising the amino acid sequence of SEQ ID NO:92, and (iii) HVR-H3 comprising the amino acid sequence of SEQ ID NO:95; and/or a light chain variable region comprising (i) HVR-L1 comprising the amino acid sequence of SEQ ID NO:98, (ii) HVR-L2 comprising the amino acid sequence of SEQ ID NO:101, and (iii) HVR-L3 comprising the amino acid sequence of SEQ ID NO:104; or
a heavy chain variable region comprising (i) HVR-H1 comprising the amino acid sequence of SEQ ID NO:90, (ii) HVR-H2 comprising the amino acid sequence of SEQ ID NO:93, and (iii) HVR-H3 comprising the amino acid sequence of SEQ ID NO:96; and/or a light chain variable region comprising (i) HVR-L1 comprising the amino acid sequence of SEQ ID NO:99, (ii) HVR-L2 comprising the amino acid sequence of SEQ ID NO: 102, and (iii) HVR-L3 comprising the amino acid sequence of SEQ ID NO:105.

In some embodiments, the light chain FR sequences comprise the following:

a) LC-FR1
(EIVLTQSPATLSLSPGERATLSC (SEQ ID NO: 48);
or
EIILTQSPATLSLSPGERATLSC (SEQ ID NO: 49));
b) LC-FR2
(WFQQKPGQAPRLLIY (SEQ ID NO: 51);
WFQQKPGQAPRLWIY (SEQ ID NO: 52);
or
WYQQKPGQAPRLLIY (SEQ ID NO: 53));
c) LC-FR3
(GIPARFSGSGSGTDFTLTISSLEPEDFAVYYC (SEQ ID NO: 55);
GVPARFSGSGSGTDYFLTISSLEPEDFAVYYC (SEQ ID NO: 56);
GVPARFSGSGSGTDFTLTISSLEPEDFAVYYC (SEQ ID NO: 57);
or
GIPARFSGSGSGTDYTLTISSLEPEDFAVYYC (SEQ ID NO: 58));
and
d) LC-FR4 (FGPGTKLDIK (SEQ ID NO: 60)).

In some embodiments, provided herein is an anti-Siglec-8 antibody (e.g., a humanized anti-Siglec-8) antibody that binds to human Siglec-8, wherein the antibody comprises a heavy chain variable region and a light chain variable region, wherein the antibody comprises:
(a) heavy chain variable domain comprising:
(1) an HC-FR1 comprising the amino acid sequence selected from SEQ ID NOs:26-29;
(2) an HVR-H1 comprising the amino acid sequence of SEQ ID NO:61;
(3) an HC-FR2 comprising the amino acid sequence selected from SEQ ID NOs:31-36;
(4) an HVR-H2 comprising the amino acid sequence of SEQ ID NO:62;
(5) an HC-FR3 comprising the amino acid sequence selected from SEQ ID NOs:38-43;
(6) an HVR-H3 comprising the amino acid sequence of SEQ ID NO:63; and
(7) an HC-FR4 comprising the amino acid sequence selected from SEQ ID NOs:45-46, and/or
(b) a light chain variable domain comprising:
(1) an LC-FR1 comprising the amino acid sequence selected from SEQ ID NOs:48-49;
(2) an HVR-L1 comprising the amino acid sequence of SEQ ID NO:64;
(3) an LC-FR2 comprising the amino acid sequence selected from SEQ ID NOs:51-53;
(4) an HVR-L2 comprising the amino acid sequence of SEQ ID NO:65;
(5) an LC-FR3 comprising the amino acid sequence selected from SEQ ID NOs:55-58;
(6) an HVR-L3 comprising the amino acid sequence of SEQ ID NO:66; and
(7) an LC-FR4 comprising the amino acid sequence of SEQ ID NO:60.

In one aspect, provided herein is an anti-Siglec-8 antibody comprising a heavy chain variable domain selected from SEQ ID NOs:2-10 and/or comprising a light chain variable domain selected from SEQ ID NOs:16-22. In one aspect, provided herein is an anti-Siglec-8 antibody comprising a heavy chain variable domain selected from SEQ ID NOs:2-14 and/or comprising a light chain variable domain selected from SEQ ID NOs:16-24. In one aspect, provided herein is an anti-Siglec-8 antibody comprising a heavy chain variable domain selected from SEQ ID NOs:2-10 and/or comprising a light chain variable domain selected from SEQ ID NO:23 or 24. In one aspect, provided herein is an anti-Siglec-8 antibody comprising a heavy chain variable domain selected from SEQ ID NOs:11-14 and/or comprising a light chain variable domain selected from SEQ ID NOs:16-22. In one aspect, provided herein is an anti-Siglec-8 antibody comprising a heavy chain variable domain selected from SEQ ID NOs:11-14 and/or comprising a light chain variable domain selected from SEQ ID NO:23 or 24. In one aspect, provided herein is an anti-Siglec-8 antibody comprising a heavy chain variable domain of SEQ ID NO:6 and/or comprising a light chain variable domain selected from SEQ ID NO:16 or 21.

In one aspect, provided herein is an anti-Siglec-8 antibody comprising a heavy chain variable domain selected from SEQ ID NOs:106-108 and/or comprising a light chain variable domain selected from SEQ ID NOs:109-111. In one aspect, provided herein is an anti-Siglec-8 antibody comprising a heavy chain variable domain of SEQ ID NO:106 and/or comprising a light chain variable domain of SEQ ID NO:109. In one aspect, provided herein is an anti-Siglec-8 antibody comprising a heavy chain variable domain of SEQ ID NO:107 and/or comprising a light chain variable domain of SEQ ID NO:110. In one aspect, provided herein is an anti-Siglec-8 antibody comprising a heavy chain variable domain of SEQ ID NO:108 and/or comprising a light chain variable domain of SEQ ID NO:111.

In some embodiments, provided herein is an anti-Siglec-8 antibody comprising a heavy chain variable domain comprising an amino acid sequence having at least 90%, 91%, 92%, 93%, 94%, 95%, 96%, 97%, 98%, or 99% sequence identity to an amino acid sequence selected from SEQ ID NOs:2-14. In some embodiments, provided herein is an anti-Siglec-8 antibody comprising a heavy chain variable domain comprising an amino acid sequence having at least 90%, 91%, 92%, 93%, 94%, 95%, 96%, 97%, 98%, or 99% sequence identity to an amino acid sequence selected from SEQ ID NOs:106-108. In some embodiments, an amino acid sequence having at least 90%, 91%, 92%, 93%, 94%, 95%, 96%, 97%, 98%, or 99% sequence identity contains substitutions, insertions, or deletions relative to the reference sequence, but an antibody comprising that amino acid sequence retains the ability to bind to human Siglec-8. In some embodiments, the substitutions, insertions, or deletions (e.g., 1, 2, 3, 4, or 5 amino acids) occur in regions outside the HVRs (i.e., in the FRs). In some embodiments, an anti-Siglec-8 antibody comprises a heavy chain variable domain comprising an amino acid sequence of SEQ ID NO:6. In some embodiments, an anti-Siglec-8 antibody comprises a heavy chain variable domain comprising an amino acid sequence selected from SEQ ID NOs:106-108.

In some embodiments, provided herein is an anti-Siglec-8 antibody comprising a light chain variable domain comprising an amino acid sequence having at least 90%, 91%, 92%, 93%, 94%, 95%, 96%, 97%, 98%, or 99% sequence identity to an amino acid sequence selected from SEQ ID NOs:16-24. In some embodiments, provided herein is an anti-Siglec-8 antibody comprising a light chain variable domain comprising an amino acid sequence having at least 90%, 91%, 92%, 93%, 94%, 95%, 96%, 97%, 98%, or 99% sequence identity to an amino acid sequence selected from SEQ ID NOs:109-111. In some embodiments, an amino acid sequence having at least 90%, 91%, 92%, 93%, 94%, 95%, 96%, 97%, 98%, or 99% sequence identity contains substitutions, insertions, or deletions relative to the reference sequence, but an antibody comprising that amino acid sequence retains the ability to bind to human Siglec-8. In some embodiments, the substitutions, insertions, or deletions (e.g., 1, 2, 3, 4, or 5 amino acids) occur in regions outside the HVRs (i.e., in the FRs). In some embodiments, an anti-Siglec-8 antibody comprises a light chain variable domain comprising an amino acid sequence of SEQ ID NO:16 or 21. In some embodiments, an anti-Siglec-8 antibody comprises a heavy chain variable domain comprising an amino acid sequence selected from SEQ ID NOs:109-111.

In one aspect, the present disclosure provides an anti-Siglec-8 antibody comprising (a) one, two, or three VH HVRs selected from those shown in Table 1 and/or (b) one, two, or three VL HVRs selected from those shown in Table 1.

In one aspect, the present disclosure provides an anti-Siglec-8 antibody comprising (a) one, two, or three VH HVRs selected from those shown in Table 2 and/or (b) one, two, or three VL HVRs selected from those shown in Table 2.

In one aspect, the present disclosure provides an anti-Siglec-8 antibody comprising (a) one, two, three or four VH FRs selected from those shown in Table 3 and/or (b) one, two, three or four VL FRs selected from those shown in Table 3.

In some embodiments, provided herein is an anti-Siglec-8 antibody comprising a heavy chain variable domain and/or a light chain variable domain of an antibody shown in Table 4, for example, HAKA antibody, HAKB antibody, HAKC antibody, etc.

TABLE 1
Amino acid sequences of HVRs of antibodies

| | | | |
|---|---|---|---|
| Antibody Chain | HVR1 | HVR2 | HVR3 |

2E2 antibody

| | | | |
|---|---|---|---|
| Heavy chain | IYGAH | VIWAGGSTNYNSALMS | DGSSPYYYSMEY |
| | SEQ ID NO: 61 | SEQ ID NO: 62 | SEQ ID NO: 63 |
| Light chain | SATSSVSYMH | STSNLAS | QQRSSYHT |
| | SEQ ID NO: 64 | SEQ ID NO: 65 | SEQ ID NO: 66 |

Humanized Heavy Chain Variants 2E2 RHA, 2E2 RHB,
2E2 RHC, 2E2 RHD, 2E2 RHE, 2E2 RHF, 2E2 RHG, 2E2
RHA2, and 2E2 RHB2

| | | | |
|---|---|---|---|
| Heavy chain | IVGAH | VIWAGGSTNYNSALMS | DGSSPYYYSMEY |
| | SEQ ID NO: 61 | SEQ ID NO: 62 | SEQ ID NO: 63 |

Humanized Light Chain Variants 2E2 RKA, 2E2 RKB,
2E2 RKC, 2E2 RKD, 2E2 RKE, 2E2 RKF, and 2E2 RKG

| | | | |
|---|---|---|---|
| Light chain | SATSSVSYMH | STSNLAS | QQRSSYPFT |
| | SEQ ID NO: 64 | SEQ ID NO: 65 | SEQ ID NO: 66 |

Humanized Heavy Chain Variants 2E2 RHES-G,
2E2 RHE E-D, 2E2 RHE Y-V, and 2E2 RHE triple

| | | | |
|---|---|---|---|
| 2E2 RHE S-G | IYGAH | VIWAGGSTNYNSALMS | DGSSPYYYGMEY |
| | SEQ ID NO: 61 | SEQ ID NO: 62 | SEQ ID NO: 67 |
| 2E2 RHE E-D | IYGAH | VIWAGGSTNYNSALMS | DGSSPYYYSMDY |
| | SEQ ID NO: 61 | SEQ ID NO: 62 | SEQ ID NO: 68 |
| 2E2 RHE Y-V | IYGAH | VIWAGGSTNYNSALMS | DGSSPYYYSMEV |
| | SEQ ID NO: 61 | SEQ ID NO: 62 | SEQ ID NO: 69 |
| 2E2 RHE triple | IYGAH | VIWAGGSTNYNSALMS | DGSSPYYYGMDV |
| | SEQ ID NO: 61 | SEQ ID NO: 62 | SEQ ID NO: 70 |

Humanized Light Chain Variants 2E2 RKA F-Y and
2E2 RKF F-Y

| | | | |
|---|---|---|---|
| 2E2 RKA F-Y | SATSSVSYMH | STSNLAS | QQRSSYPYT |
| | SEQ ID NO: 64 | SEQ ID NO: 65 | SEQ ID NO: 71 |
| 2E2 RKF F-Y | SATSSVSYMH | STSNLAS | QQRSSYPYT |
| | SEQ ID NO: 64 | SEQ ID NO: 65 | SEQ ID NO: 71 |

TABLE 2
Amino acid sequences of HVRs from murine
1C3, 1H10, and 4F11 antibodies

| Antibody | Chain | HVR1 | HVR2 | HVR3 |
|---|---|---|---|---|
| 1C3 | Heavy Chain | SYAMS | IISSGGSYTYYSDSVKG | HETAQAAWFAY |
| | | SEQ ID NO: 88 | SEQ ID NO: 91 | SEQ ID NO: 94 |
| 1H10 | Heavy Chain | DYYMY | RIAPEDGDTEYAPKFQG | EGNYYGSSILDY |
| | | SEQ ID NO: 89 | SEQ ID NO: 92 | SEQ ID NO: 95 |
| 4F11 | Heavy Chain | SSWMN | QIYPGDDYTNYNGKFKG | LGPYGPFAD |
| | | SEQ ID NO: 90 | SEQ ID NO: 93 | SEQ ID NO: 96 |
| 1C3 | Light Chain | SASSSVSYMH | DTSKLAY | QQWSSNPPT |
| | | SEQ ID NO: 97 | SEQ ID NO: 100 | SEQ ID NO: 103 |
| 1H10 | Light Chain | RASQDITNYLN | FTSRLHS | QQGNTLPWT |
| | | SEQ ID NO: 98 | SEQ ID NO: 101 | SEQ ID NO: 104 |
| 4F11 | Light Chain | SASSSVSYMY | DTSSLAS | QQWNSDPYT |
| | | SEQ ID NO: 99 | SEQ ID NO: 102 | SEQ ID NO: 105 |

TABLE 3
Amino acid sequences of FRs of antibodies

| | FR1 | FR2 | FR3 | FR4 |
|---|---|---|---|---|
| Heavy Chain | | | | |
| 2E2 | QVQLKESGPGLVA | WVRQPPGKGLEW | RLSISKDNSKSQVF | WGQGTSVTVSS |
| | PSQSLSITCTVSGFS | LG | LKINSLQTDDTAL | (SEQ ID NO: 44) |
| | LT | (SEQ ID NO: 30) | YYCAR | |
| | (SEQ ID NO: 25) | | (SEQ ID NO: 37) | |
| 2E2 RHA | EVQLVESGGGLVQ | WVRQAPGKGLEW | RFTISKDNSKNTVY | WGQGTTVTVSS |
| | PGGSLRLSCAASGF | VS | LQMNSLRAEDTAV | (SEQ ID NO: 45) |
| | SLT | (SEQ ID NO: 31) | YYCAR | |
| | (SEQ ID NO: 26) | | (SEQ ID NO: 38) | |
| 2E2 RHB | EVQLVESGGGLVQ | WVRQAPGKGLEW | RLSISKDNSKNTVY | WGQGTTVTVSS |
| | PGGSLRLSCAVSGF | LG | LQMNSLRAEDTAV | (SEQ ID NO: 45) |
| | SLT | (SEQ ID NO: 32) | YYCAR | |
| | (SEQ ID NO: 27) | | (SEQ ID NO: 39) | |
| 2E2 RHC | EVQLVESGGGLVQ | WVRQAPGKGLEW | RFTISKDNSKNTVY | WGQGTTVTVSS |
| | PGGSLRLSCAVSGF | VS | LQMNSLRAEDTAV | (SEQ ID NO: 45) |
| | SLT | (SEQ ID NO: 31) | YYCAR | |
| | (SEQ ID NO: 27) | | (SEQ ID NO: 38) | |
| 2E2 RHD | EVQLVESGGGLVQ | WVRQAPGKGLEW | RFTISKDNSKNTVY | WGQGTTVTVSS |
| | PGGSLRLSCAASGF | LS | LQMNSLRAEDTAV | (SEQ ID NO: 45) |
| | SLT | (SEQ ID NO: 33) | YYCAR | |
| | (SEQ ID NO: 26) | | (SEQ ID NO: 38) | |
| 2E2 RHE | EVQLVESGGGLVQ | WVRQAPGKGLEW | RFTISKDNSKNTVY | WGQGTTVTVSS |
| | PGGSLRLSCAASGF | VG | LQMNSLRAEDTAV | (SEQ ID NO: 45) |
| | SLT | (SEQ ID NO: 34) | YYCAR | |
| | (SEQ ID NO: 26) | | (SEQ ID NO: 38) | |
| 2E2 RHF | EVQLVESGGGLVQ | WVRQAPGKGLEW | RLTISKDNSKNTV | WGQGTTVTVSS |
| | PGGSLRLSCAASGF | VS | YLQMNSLRAEDTA | (SEQ ID NO: 45) |
| | SLT | (SEQ ID NO: 31) | VVYCAR | |
| | (SEQ ID NO: 26) | | (SEQ ID NO: 40) | |
| 2E2 RHG | EVQLVESGGGLVQ | WVRQAPGKGLEW | RFSISKDNSKNTVY | WGQGTTVTVSS |
| | PGGSLRLSCAASGF | VS | LQMNSLRAEDTAV | (SEQ ID NO: 45) |
| | SLT | (SEQ ID NO: 31) | YYCAR | |
| | (SEQ ID NO: 26) | | (SEQ ID NO: 41) | |
| 2E2 RHA2 | QVQLQESGPGLVK | WIRQPPGKGLEWI | RVTISVDTSKNQFS | WGQGTLVTVSS |
| | PSETLSLTCTVSGG | G | LKLSSVTAADTAV | (SEQ ID NO: 46) |
| | SIS | (SEQ ID NO: 35) | YYCAR | |
| | (SEQ ID NO: 28) | | (SEQ ID NO: 42) | |
| 2E2 RHB2 | QVQLQESGPGLVK | WVRQPPGKGLEW | RLSISKDNSKNQVS | WGQGTLVTVSS |
| | PSETLSLTCTVSGF | LG | LKLSSVTAADTAV | (SEQ ID NO: 46) |
| | SLT | (SEQ ID NO: 36) | YYCAR | |
| | (SEQ ID NO: 29) | | (SEQ ID NO: 43) | |
| 2E2 RHE S-G | EVQLVESGGGLVQ | WVRQAPGKGLEW | RFTISKDNSKNTVY | WGQGTTVTVSS |
| | PGGSLRLSCAASGF | VG | LQMNSLRAEDTAV | (SEQ ID NO: 45) |
| | SLT | (SEQ ID NO: 34) | YYCAR | |
| | (SEQ ID NO: 26) | | (SEQ ID NO: 38) | |
| 2E2 RHE E-D | EVQLVESGGGLVQ | WVRQAPGKGLEW | RFTISKDNSKNTVY | WGQGTTVTVSS |
| | PGGSLRLSCAASGF | VG | LQMNSLRAEDTAV | (SEQ ID NO: 45) |
| | SLT | (SEQ ID NO: 34) | YYCAR | |
| | (SEQ ID NO: 26) | | (SEQ ID NO: 38) | |
| 2E2 RHE Y-V | EVQLVESGGGINQ | WVRQAPGKGLEW | RFTISKDNSKNTVY | WGQGTTVTVSS |
| | PGGSLRLSCAASGF | VG | LQMNSLRAEDTAV | (SEQ ID NO: 45) |
| | SLT | (SEQ ID NO: 34) | YYCAR | |
| | (SEQ ID NO: 26) | | (SEQ ID NO: 38) | |
| 2E2 RHE | EVQLVESOGGLVQ | WVRQAPGKGLEW | RFTISKDNSKNTVY | WGQGTTVTVSS |
| triple | PGGSLRLSCAASGF | VG | LQMNSLRAEDTAV | (SEQ ID NO: 45) |
| | SLT | (SEQ ID NO: 34) | YYCAR | |
| | (SEQ ID NO: 26) | | (SEQ ID NO: 38) | |
| Light Chain | | | | |
| 2E2 | QIILTQSPAIMSASP | WFQQKPGTSPKLW | GVPVRFSGSGSGTS | FGSGTKLEIK |
| | GEKVSITC | IY | YSLTISRMEAEDA | (SEQ ID NO: 59) |
| | (SEQ ID NO: 47) | (SEQ ID NO: 50) | ATYYC | |
| | | | (SEQ ID NO: 54) | |
| RKA | EIVLTQSPATLSLSP | WFQQKPGQAPRLL | GIPARFSGSGSGTD | FGPGTKLDIK |
| | GERATLSC | IY | FTLTISSLEPEDFAV | (SEQ ID NO: 60) |
| | (SEQ ID NO: 48) | (SEQ ID NO: 51) | YYC | |
| | | | (SEQ ID NO: 55) | |
| RKB | EIILTQSPATLSLSP | WFQQKPGQAPRL | GVPARFSGSGSGT | FGPGTKLDIK |
| | GERATLSC | WIY | DYTLTISSLEPEDF | (SEQ ID NO: 60) |
| | (SEQ ID NO: 49) | (SEQ ID NO: 52) | AVYYC | |
| | | | (SEQ ID NO: 56) | |
| RKC | EIILTQSPATLSLSP | WFQQKPGQAPRLL | GIPARFSGSGSGTD | FGPGTKLDIK |
| | GERATLSC | IY | FTLTISSLEPEDFAV | (SEQ ID NO: 60) |
| | (SEQ ID NO: 49) | (SEQ ID NO: 51) | YYC | |
| | | | (SEQ ID NO: 55) | |
| RKD | EIVLTQSPATLSLSP | WFQQKPGQAPRL | GIPARFSGSGSGTD | FGPGTKLDIK |
| | GERATLSC | WIY | FTLTISSLEPEDFAV | (SEQ ID NO: 60) |
| | (SEQ ID NO: 48) | (SEQ ID NO: 52) | YYC | |
| | | | (SEQ ID NO: 55) | |
| RKE | EWLTQSPATLSLSP | WFQQKPGQAPRLL | GVPARFSGSGSGT | FGPGTKLDIK |
| | GERATLSC | FY | DFTLTISSLEPEDFA | (SEQ ID NO: 60) |
| | (SEQ ID NO: 48) | (SEQ ID NO: 51) | VYYC | |
| | | | (SEQ ID NO: 57) | |
| RKF | EIVLTQSPATLSLSP | WFQQKPGQAPRLL | GIPARFSGSGSGTD | FGPGIRLDIK |
| | GERATLSC | IY | YFLTISSLEPEDEA | (SEQ ID NO: 60) |
| | (SEQ ID NO: 48) | (SEQ ID NO: 51) | VYYC | |
| | | | (SEQ ID NO: 58) | |
| RKG | EIVLTQSPATLSLSP | WYQQKPGQAPRL | GIPARESGSGSGTD | FGPGTKLDIK |
| | GERATLSC | LIY | FTLTISSLEPEDFAV | (SEQ ID NO: 60) |
| | (SEQ ID NO: 48) | (SEQ ID NO: 53) | YYC | |
| | | | (SEQ ID NO: 55) | |
| RKA F-Y | EIVLTQSPATLSLSP | WFQQKPGQAPRLL | GIPARFSGSGSGTD | FGPGTKLDIK |
| | GERATLSC | IY | FTLTISSLEPEDEAV | (SEQ ID NO: 60) |
| | (SEQ ID NO: 48) | (SEQ ID NO: 51) | YYC | |
| | | | (SEQ ID NO: 55) | |
| RKF F-Y | EIVLTQSPATLSLSP | WFQQKPGQAPRLL | GIPARFSGSGSGTD | FGPGTKLDIK |
| | GERATLSC | IY | YTLTISSLEPEDFA | (SEQ ID NO: 60) |
| | (SEQ ID NO: 48) | (SEQ ID NO: 51) | VYYC | |
| | | | (SEQ ID NO: 58) | |

TABLE 4
Amino acid sequences of variable regions of antibodies

| Antibody Name | Variable Heavy Chain | Variable Light Chain |
|---|---|---|
| ch2C4 | ch2C4 VH | ch2C4 VK |
| ch2E2 | ch2E2 VH (SEQ ID NO: 1) | ch2E2 VK (SEQ ID NO: 15) |
| cVHKA | ch2E2 VH (SEQ ID NO: 1) | 2E2 RKA (SEQ ID NO: 16) |
| cVHKB | ch2E2 VH (SEQ ID NO: 1) | 2E2 RKB (SEQ ID NO: 17) |
| HAcVK | 2E2 RHA (SEQ ID NO: 2) | ch2E2 VK (SEQ ID NO: 15) |
| HBcVK | 2E2 RHB (SEQ ID NO: 3) | ch2E2 VK (SEQ ID NO: 15) |
| HAKA | 2E2 RHA (SEQ ID NO: 2) | 2E2 RKA (SEQ ID NO: 16) |
| HAKB | 2E2 RHA (SEQ ID NO: 2) | 2E2 RKB (SEQ ID NO: 17) |
| HAKC | 2E2 RHA (SEQ ID NO: 2) | 2E2 RKC (SEQ ID NO: 18) |
| HAKD | 2E2 RHA (SEQ ID NO: 2) | 2E2 RKD (SEQ ID NO: 19) |
| HAKE | 2E2 RHA (SEQ ID NO: 2) | 2E2 RKE (SEQ ID NO: 20) |
| HAKF | 2E2 RHA (SEQ ID NO: 2) | 2E2 RKF (SEQ ID NO: 21) |
| HAKG | 2E2 RHA (SEQ ID NO: 2) | 2E2 RKG (SEQ ID NO: 22) |
| HBKA | 2E2 RHB (SEQ ID NO: 3) | 2E2 RKA (SEQ ID NO: 16) |
| HBKB | 2E2 RHB (SEQ ID NO: 3) | 2E2 RKB (SEQ ID NO: 17) |
| HBKC | 2E2 RHB (SEQ ID NO: 3) | 2E2 RKC (SEQ ID NO: 18) |
| HBKD | 2E2 RHB (SEQ ID NO: 3) | 2E2 RKD (SEQ ID NO: 19) |
| HBKE | 2E2 RHB (SEQ ID NO: 3) | 2E2 RKE (SEQ ID NO: 20) |
| HBKF | 2E2 RHB (SEQ ID NO: 3) | 2E2 RKF (SEQ ID NO: 21) |
| HBKG | 2E2 RHB (SEQ ID NO: 3) | 2E2 RKG (SEQ ID NO: 22) |
| HCKA | 2E2 RHC (SEQ ID NO: 4) | 2E2 RKA (SEQ ID NO: 16) |
| HCKB | 2E2 RHC (SEQ ID NO: 4) | 2E2 RKB (SEQ ID NO: 17) |
| HCKC | 2E2 RHC (SEQ ID NO: 4) | 2E2 RKC (SEQ ID NO: 18) |
| HCKD | 2E2 RHC (SEQ ID NO: 4) | 2E2 RKD (SEQ ID NO: 19) |
| HCKE | 2E2 RHC (SEQ ID NO: 4) | 2E2 RKE (SEQ ID NO: 20) |
| HCKF | 2E2 RHC (SEQ ID NO: 4) | 2E2 RKF (SEQ ID NO: 21) |
| HCKG | 2E2 RHC (SEQ ID NO: 4) | 2E2 RKG (SEQ ID NO: 22) |
| HDKA | 2E2 RHD (SEQ ID NO: 5) | 2E2 RKA (SEQ ID NO: 16) |
| HDKB | 2E2 RHD (SEQ ID NO: 5) | 2E2 RKB (SEQ ID NO: 17) |
| HDKC | 2E2 RHD (SEQ ID NO: 5) | 2E2 RKC (SEQ ID NO: 18) |
| HDKD | 2E2 RHD (SEQ ID NO: 5) | 2E2 RKD (SEQ ID NO: 19) |
| HDKE | 2E2 RHD (SEQ ID NO: 5) | 2E2 RKE (SEQ ID NO: 20) |
| HDKF | 2E2 RHD (SEQ ID NO: 5) | 2E2 RKF (SEQ ID NO: 21) |
| HDKG | 2E2 RHD (SEQ ID NO: 5) | 2E2 RKG (SEQ ID NO: 22) |
| HEKA | 2E2 RHE (SEQ ID NO: 6) | 2E2 RKA (SEQ ID NO: 16) |
| HEKB | 2E2 RHE (SEQ ID NO: 6) | 2E2 RKB (SEQ ID NO: 17) |
| HEKC | 2E2 RHE (SEQ ID NO: 6) | 2E2 RKC (SEQ ID NO: 18) |
| HEKD | 2E2 RHE (SEQ ID NO: 6) | 2E2 RKD (SEQ ID NO: 19) |
| HEKE | 2E2 RHE (SEQ ID NO: 6) | 2E2 RKE (SEQ ID NO: 20) |
| HEKF | 2E2 RHE (SEQ ID NO: 6) | 2E2 RKF (SEQ ID NO: 21) |
| HEKG | 2E2 RHE (SEQ ID NO: 6) | 2E2 RKG (SEQ ID NO: 22) |
| HFKA | 2E2 RHF (SEQ ID NO: 7) | 2E2 RKA (SEQ ID NO: 16) |
| HFKB | 2E2 RHF (SEQ ID NO: 7) | 2E2 RKB (SEQ ID NO: 17) |
| HFKC | 2E2 RHF (SEQ ID NO: 7) | 2E2 RKC (SEQ ID NO: 18) |
| HFKD | 2E2 RHF (SEQ ID NO: 7) | 2E2 RKD (SEQ ID NO: 19) |
| HFKE | 2E2 RHF (SEQ ID NO: 7) | 2E2 RKE (SEQ ID NO: 20) |
| HFKF | 2E2 RHF (SEQ ID NO: 7) | 2E2 RKF (SEQ ID NO: 21) |
| HFKG | 2E2 RHF (SEQ ID NO: 7) | 2E2 RKG (SEQ ID NO: 22) |
| HGKA | 2E2 RHG (SEQ ID NO: 8) | 2E2 RKA (SEQ ID NO: 16) |
| HGKB | 2E2 RHG (SEQ ID NO: 8) | 2E2 RKB (SEQ ID NO: 17) |
| HGKC | 2E2 RHG (SEQ ID NO: 8) | 2E2 RKC (SEQ ID NO: 18) |
| HGKD | 2E2 RHG (SEQ ID NO: 8) | 2E2 RKD (SEQ ID NO: 19) |
| HGKE | 2E2 RHG (SEQ ID NO: 8) | 2E2 RKE (SEQ ID NO: 20) |
| HGKF | 2E2 RHG (SEQ ID NO: 8) | 2E2 RKF (SEQ ID NO: 21) |
| HGKG | 2E2 RHG (SEQ ID NO: 8) | 2E2 RKG (SEQ ID NO: 22) |
| HA2KA | 2E2 RHA2 (SEQ ID NO: 9) | 2E2 RKA (SEQ ID NO: 16) |
| HA2KB | 2E2 RHA2 (SEQ ID NO: 9) | 2E2 RKB (SEQ ID NO: 17) |
| HB2KA | 2E2 RHB2 (SEQ ID NO: 10) | 2E2 RKA (SEQ ID NO: 16) |
| HB2KB | 2E2 RHB2 (SEQ ID NO: 10) | 2E2 RKB (SEQ ID NO: 17) |
| HA2KF | 2E2 RHA2 (SEQ ID NO: 9) | 2E2 RKF (SEQ ID NO: 21) |
| HB2KF | 2E2 RHB2 (SEQ ID NO: 10) | 2E2 RKF (SEQ ID NO: 21) |
| HA2KC | 2E2 RHA2 (SEQ ID NO: 9) | 2E2 RKC (SEQ ID NO: 18) |
| HA2KD | 2E2 RHA2 (SEQ ID NO: 9) | 2E2 RKD (SEQ ID NO: 19) |
| HA2KE | 2E2 RHA2 (SEQ ID NO: 9) | 2E2 RKE (SEQ ID NO: 20) |
| HA2KF | 2E2 RHA2 (SEQ ID NO: 9) | 2E2 RKF (SEQ ID NO: 21) |
| HA2KG | 2E2 RHA2 (SEQ ID NO: 9) | 2E2 RKG (SEQ ID NO: 22) |
| HB2KC | 2E2 RHB2 (SEQ ID NO: 10) | 2E2 RKC (SEQ ID NO: 18) |
| HB2KD | 2E2 RHB2 (SEQ ID NO: 10) | 2E2 RKD (SEQ ID NO: 19) |
| HB2KE | 2E2 RHB2 (SEQ ID NO: 10) | 2E2 RKE (SEQ ID NO: 20) |
| HA2KFmut | 2E2 RHA2 (SEQ ID NO: 9) | 2E2 RKF F-Y mut (SEQ ID NO: 24) |
| HB2KFmut | 2E2 RHB2 (SEQ ID NO: 10) | 2E2 RKF F-Y mut (SEQ ID NO: 24) |
| HEKAmut | 2E2 RHE (SEQ ID NO: 6) | 2E2 RKA F-Y mut (SEQ ID NO: 23) |
| HEKFmut | 2E2 RHE (SEQ ID NO: 6) | 2E2 RKF F-Y mut (SEQ ID NO: 24) |
| HAKFmut | 2E2 RHA (SEQ ID NO: 2) | 2E2 RKF F-Y mut (SEQ ID NO: 24) |
| HBKFmut | 2E2 RHB (SEQ ID NO: 3) | 2E2 RKF F-Y mut (SEQ ID NO: 24) |
| HCKFmut | 2E2 RHC (SEQ ID NO: 4) | 2E2 RKF F-Y mut (SEQ ID NO: 24) |
| HDKFmut | 2E2 RHD (SEQ ID NO: 5) | 2E2 RKF F-Y mut (SEQ ID NO: 24) |
| HFKFmut | 2E2 RHF (SEQ ID NO: 7) | 2E2 RKF F-Y mut (SEQ ID NO: 24) |
| HGKFmut | 2E2 RHG (SEQ ID NO: 8) | 2E2 RKF F-Y mut (SEQ ID NO: 24) |
| RHE Y-VKA | 2E2 RHE Y-V (SEQ ID NO: 13) | 2E2 RKA (SEQ ID NO: 16) |
| RHE Y-VKB | 2E2 RHE Y-V (SEQ ID NO: 13) | 2E2 RKB (SEQ ID NO: 17) |
| RHE Y-VKC | 2E2 RHE Y-V (SEQ ID NO: 13) | 2E2 RKC (SEQ ID NO: 18) |
| RHE Y-VKD | 2E2 RHE Y-V (SEQ ID NO: 13) | 2E2 RKD (SEQ ID NO: 19) |
| RHE Y-VKE | 2E2 RHE Y-V (SEQ ID NO: 13) | 2E2 RKE (SEQ ID NO: 20) |
| RHE Y-VKF | 2E2 RHE Y-V (SEQ ID NO: 13) | 2E2 RKF (SEQ ID NO: 21) |
| RHE Y-VKG | 2E2 RHE Y-V (SEQ ID NO: 13) | 2E2 RKG (SEQ ID NO: 22) |
| RHE E-DKA | 2E2 RHE E-D (SEQ ID NO: 12) | 2E2 RKA (SEQ ID NO: 16) |
| RHE E-DKB | 2E2 RHE E-D (SEQ ID NO: 12) | 2E2 RKB (SEQ ID NO: 17) |
| RHE E-DKC | 2E2 RHE E-D (SEQ ID NO: 12) | 2E2 RKC (SEQ ID NO: 18) |
| RHE E-DKD | 2E2 RHE E-D (SEQ ID NO: 12) | 2E2 RKD (SEQ ID NO: 19) |
| RHE E-DKE | 2E2 RHE E-D (SEQ ID NO: 12) | 2E2 RKE (SEQ ID NO: 20) |
| RHE E-DKF | 2E2 RHE E-D (SEQ ID NO: 12) | 2E2 RKF (SEQ ID NO: 21) |
| RHE E-DKG | 2E2 RHE E-D (SEQ ID NO: 12) | 2E2 RKG (SEQ ID NO: 22) |
| RHE E-DKFmut | 2E2 RHE E-D (SEQ ID NO: 12) | 2E2 RKF F-Y mut (SEQ ID NO: 24) |
| RHE S-GKA | 2E2 RHE S-G (SEQ ID NO: 11) | 2E2 RKA (SEQ ID NO: 16) |
| RHE S-GKB | 2E2 RHE S-G (SEQ ID NO: 11) | 2E2 RKB (SEQ ID NO: 17) |
| RHE S-GKC | 2E2 RHE S-G (SEQ ID NO: 11) | 2E2 RKC (SEQ ID NO: 18) |
| RHE S-GKD | 2E2 RHE S-G (SEQ ID NO: 11) | 2E2 RKD (SEQ ID NO: 19) |
| RHE S-GKE | 2E2 RHE S-G (SEQ ID NO: 11) | 2E2 RKE (SEQ ID NO: 20) |
| RHE S-GKF | 2E2 RHE S-G (SEQ ID NO: 11) | 2E2 RKF (SEQ ID NO: 21) |
| RHE S-GKG | 2E2 RHE S-G (SEQ ID NO: 11) | 2E2 RKG (SEQ ID NO: 22) |
| RHE Triple-KA | 2E2 RHE triple (SEQ ID NO: 14) | 2E2 RKA (SEQ ID NO: 16) |
| RHE Triple-KB | 2E2 RHE triple (SEQ ID NO: 14) | 2E2 RKB (SEQ ID NO: 17) |
| RHE Triple-KC | 2E2 RHE triple (SEQ ID NO: 14) | 2E2 RKC (SEQ ID NO: 18) |
| RHE Triple-KD | 2E2 RHE triple (SEQ ID NO: 14) | 2E2 RKD (SEQ ID NO: 19) |
| RHE Triple-KE | 2E2 RHE triple (SEQ ID NO: 14) | 2E2 RKE (SEQ ID NO: 20) |
| RHE Triple-KF | 2E2 RHE triple (SEQ ID NO: 14) | 2E2 RKF (SEQ ID NO: 21) |
| RHE Triple-KG | 2E2 RHE triple (SEQ ID NO: 14) | 2E2 RKG (SEQ ID NO: 22) |
| RHE Triple-KFmut | 2E2 RHE triple (SEQ ID NO: 14) | 2E2 RKF F-Y mut (SEQ ID NO: 24) |
| RHE Y-VKFmut | 2E2 RHE Y-V (SEQ ID NO: 13) | 2E2 RKF F-Y mut (SEQ ID NO: 24) |
| RHE E-DKFmut | 2E2 RHE E-D (SEQ ID NO: 12) | 2E2 RKF F-Y mut (SEQ ID NO: 24) |

There are five classes of immunoglobulins: IgA, IgD, IgE, IgG and IgM, having heavy chains designated α, δ, ε, γ and μ, respectively. The γ and α classes are further divided into subclasses e.g., humans express the following subclasses: IgG1, IgG2, IgG3, IgG4, IgA1 and IgA2. IgG1 antibodies can exist in multiple polymorphic variants termed allotypes (reviewed in Jefferis and Lefranc 2009. mAbs Vol 1 Issue 4 1-7) any of which are suitable for use in some of the embodiments herein. Common allotypic variants in human populations are those designated by the letters a,f,n,z or combinations thereof. In any of the embodiments herein, the antibody may comprise a heavy chain Fc region comprising a human IgG Fc region. In further embodiments, the human IgG Fc region comprises a human IgG1 or IgG4. In some embodiments, the antibody is an IgG1 antibody. In some embodiments, the antibody is an IgG4 antibody. In some embodiments, the human IgG4 comprises the amino acid substitution S228P, wherein the amino acid residues are numbered according to the EU index as in Kabat. In some embodiments, the human IgG1 comprises the amino acid sequence of SEQ ID NO:78. In some embodiments, the human IgG4 comprises the amino acid sequence of SEQ ID NO:79.

In some embodiments, provided herein is an anti-Siglec-8 antibody comprising a heavy chain comprising the amino acid sequence of SEQ ID NO:75; and/or a light chain comprising the amino acid sequence selected from SEQ ID NOs:76 or 77. In some embodiments, the antibody may comprise a heavy chain comprising the amino acid sequence of SEQ ID NO:87; and/or a light chain comprising the amino acid sequence of SEQ ID NO:76. In some embodiments, the anti-Siglec-8 antibody induces apoptosis of activated eosinophils. In some embodiments, the anti-Siglec-8 antibody induces apoptosis of resting eosinophils. In some embodiments, the anti-Siglec-8 antibody depletes activated eosinophils and inhibits mast cell activation. In some embodiments, the anti-Siglec-8 antibody depletes or reduces mast cells and inhibits mast cell activation. In some embodiments, the anti-Siglec-8 antibody depleted or reduces the number of mast cells. In some embodiments, the anti-Siglec-8 antibody kills mast cells by ADCC activity. In some embodiments, the antibody depletes or reduces mast cells expressing Siglec-8 in a tissue. In some embodiments, the antibody depletes or reduces mast cells expressing Siglec-8 in a biological fluid.

1. Antibody Affinity

In some aspects, an anti-Siglec-8 antibody described herein binds to human Siglec-8 with about the same or higher affinity and/or higher avidity as compared to mouse antibody 2E2 and/or mouse antibody 2C4. In certain embodiments, an anti-Siglec-8 antibody provided herein has a dissociation constant (Kd) of ≤1 μM, ≤150 nM, ≤100 nM, ≤50 nM, ≤10 nM, ≤1 nM, ≤0.1 nM, ≤0.01 nM, or: 0.001 nM (e.g. 10-8 M or less, e.g. from 10-8 M to 10-13 M, e.g., from 10-9 M to 10-13 M). In some embodiments, an anti-Siglec-8 antibody described herein binds to human Siglec-8 at about 1.5-fold, about 2-fold, about 3-fold, about 4-fold, about 5-fold, about 6-fold, about 7-fold, about 8-fold, about 9-fold or about 10-fold higher affinity than mouse antibody 2E2 and/or mouse antibody 2C4. In some embodiments, the anti-Siglec-8 antibody comprises a heavy chain variable region comprising the amino acid sequence of SEQ ID NO:6; and/or a light chain variable region comprising the amino acid sequence selected from SEQ ID NOs:16 or 21.

In one embodiment, the binding affinity of the anti-Siglec-8 antibody can be determined by a surface plasmon resonance assay. For example, the Kd or Kd value can be measured by using a BIAcore™-2000 or a BIAcore™-3000 (BIAcore, Inc., Piscataway. N.J.) at 25° C. with immobilized antigen CM5 chips at ˜10 response units (RU). Briefly, carboxymethylated dextran biosensor chips (CM5, BIAcore®, Inc.) are activated with N-ethyl-N′-(3-dimethylaminopropyl)-carbodiimide hydrochloride (EDC) and N-hydroxysuccinimide (NHS) according to the supplier's instructions. Capture antibodies (e.g., anti-human-Fc) are diluted with 10 mM sodium acetate, pH 4.8, before injection at a flow rate of 30 μl/minute and further immobilized with an anti-Siglec-8 antibody. For kinetics measurements, two-fold serial dilutions of dimeric Siglec-8 are injected in PBS with 0.05% Tween 20 (PBST) at 25° C. at a flow rate of approximately 25 μl/min. Association rates (kₒₙ) and dissociation rates (k_{off}) are calculated using a simple one-to-one Langmuir binding model (BIAcore® Evaluation Software version 3.2) by simultaneously fitting the association and dissociation sensorgrams. The equilibrium dissociation constant (Kd) is calculated as the ratio koff/kon. See, e.g., Chen, Y., et al., (1999) J. Mol. Biol. 293:865-881.

In another embodiment, biolayer interferometry may be used to determine the affinity of anti-Siglec-8 antibodies against Siglec-8. In an exemplary assay, Siglec-8-Fc tagged protein is immobilized onto anti-human capture sensors, and incubated with increasing concentrations of mouse, chimeric, or humanized anti-Siglec-8 Fab fragments to obtain affinity measurements using an instrument such as, for example, the Octet Red 384 System (ForteBio).

The binding affinity of the anti-Siglec-8 antibody can, for example, also be determined by the Scatchard analysis described in Munson et al., Anal. Biochem., 107:220 (1980) using standard techniques well known in the relevant art. See also Scatchard, G., Ann. N.Y. Acad. Sci. 51:660 (1947).

2. Antibody Avidity

In some embodiments, the binding avidity of the anti-Siglec-8 antibody can be determined by a surface plasmon resonance assay. For example, the Kd or Kd value can be measured by using a BIAcore T100. Capture antibodies (e.g., goat-anti-human-Fc and goat-anti-mouse-Fc) are immobilized on a CM5 chip. Flow-cells can be immobilized with anti-human or with anti-mouse antibodies. The assay is conducted at a certain temperature and flow rate, for example, at 25° C. at a flow rate of 30 μl/min. Dimeric Siglec-8 is diluted in assay buffer at various concentrations, for example, at a concentration ranging from 15 nM to 1.88 pM. Antibodies are captured and high performance injections are conducted, followed by dissociations. Flow cells are regenerated with a buffer, for example, 50 mM glycine pH 1.5. Results are blanked with an empty reference cell and multiple assay buffer injections, and analyzed with 1:1 global fit parameters.

3. Competition Assays

Competition-assays can be used to determine whether two antibodies bind the same epitope by recognizing identical or sterically overlapping epitopes or one antibody competitively inhibits binding of another antibody to the antigen. These assays are known in the art. Typically, antigen or antigen expressing cells is immobilized on a multi-well plate and the ability of unlabeled antibodies to block the binding of labeled antibodies is measured. Common labels for such competition assays are radioactive labels or enzyme labels. In some embodiments, an anti-Siglec-8 antibody described herein competes with a 2E2 antibody described herein, for binding to the epitope present on the cell surface of a cell (e.g., a mast cell). In some embodiments, an anti-Siglec-8 antibody described herein competes with an antibody comprising a heavy chain variable domain comprising the amino acid sequence of SEQ ID NO:1, and a light chain variable region comprising the amino acid sequence of SEQ ID NO:15, for binding to the epitope present on the cell surface of a cell (e.g., a mast cell). In some embodiments, an anti-Siglec-8 antibody described herein competes with a 2C4 antibody described herein, for binding to the epitope present on the cell surface of a cell (e.g., a mast cell). In some embodiments, an anti-Siglec-8 antibody described herein competes with an antibody comprising a heavy chain variable domain comprising the amino acid sequence of SEQ ID NO:2 (as found in U.S. Pat. No. 8,207,305), and a light chain variable region comprising the amino acid sequence of SEQ ID NO:4 (as found in U.S. Pat. No. 8,207,305), for binding to the epitope present on the cell surface of a cell (e.g., a mast cell).

4. Thermal Stability

In some aspects, an anti-Siglec-8 described herein has a melting temperature (Tm) of at least about 70° C., at least about 71° C., or at least about 72° C. in a thermal shift assay. In an exemplary thermal shift assay, samples comprising a humanized anti-Siglec-8 antibody are incubated with a fluorescent dye (Sypro Orange) for 71 cycles with 1° C. increase per cycle in a qPCR thermal cycler to determine the Tm. In some embodiments, the anti-Siglec-8 antibody has a similar or higher Tm as compared to mouse 2E2 antibody and/or mouse 2C4 antibody. In some embodiments, the anti-Siglec-8 antibody comprises a heavy chain variable region comprising the amino acid sequence of SEQ ID NO:6; and/or a light chain variable region comprising the amino acid sequence selected from SEQ ID NOs:16 or 21. In some embodiments, the anti-Siglec-8 antibody has the same or higher Tm as compared to a chimeric 2C4 antibody. In some embodiments, the anti-Siglec-8 antibody has the same or higher Tm as compared to an antibody having a heavy chain comprising the amino acid sequence of SEQ ID NO:84 and a light chain comprising the amino acid sequence of SEQ ID NO:85.

5. Biological Activity Assays

In some embodiments, an anti-Siglec-8 antibody described herein depletes eosinophils and inhibits mast cells. Assays for assessing apoptosis of cells are well known in the art, for example staining with Annexin V and the TUNNEL assay.

In some embodiments, an anti-Siglec-8 antibody described herein induces ADCC activity. In some embodiments, an anti-Siglec-8 antibody described herein kills eosinophils expressing Siglec-8 by ADCC activity. In some embodiments, a composition comprises non-fucosylated (i.e., afucosylated) anti-Siglec-8 antibodies. In some embodiments, a composition comprising non-fucosylated anti-Siglec-8 antibodies described herein enhances ADCC activity against Siglec-8 expressing eosinophils as compared to a composition comprising partially fucosylated anti-Siglec-8 antibodies. Assays for assessing ADCC activity are well known in the art and described herein. In an exemplary assay, to measure ADCC activity, effector cells and target cells are used. Examples of effector cells include natural killer (NK) cells, large granular lymphocytes (LGL), lymphokine-activated killer (LAK) cells and PBMC comprising NK and LGL, or leukocytes having Fc receptors on the cell surfaces, such as neutrophils, eosinophils and macrophages. Effector cells can be isolated from any source including individuals with a disease of interest (e.g., mast cell gastritis, mast cell esophagitis, mast cell colitis, mast cell enteritis, mast cell duodenitis, and/or mast cell gastroenteritis). The target cell is any cell which expresses on the cell surface antigens that antibodies to be evaluated can recognize. An example of such a target cell is an eosinophil which expresses Siglec-8 on the cell surface. Another example of such a target cell is a cell line (e.g., Ramos cell line) which expresses Siglec-8 on the cell surface (e.g., Ramos 2C10)). Target cells can be labeled with a reagent that enables detection of cytolysis. Examples of reagents for labeling include a radio-active substance such as sodium chromate (Na₂⁵¹CrO₄). See, e.g., Immunology, 14, 181 (1968); J. Immunol. Methods., 172, 227 (1994); and J. Immunol. Methods., 184, 29 (1995).

In an exemplary assay to assess ADCC and apoptotic activity of anti-Siglec-8 antibodies on mast cells, human mast cells are isolated from human tissues or biological fluids according to published protocols (Guhl et al., Biosci. Biotechnol. Biochem., 2011, 75:382-384; Kulka et al., In Current Protocols in Immunology, 2001, (John Wiley & Sons, Inc.)) or differentiated from human hematopoietic stem cells, for example as described by Yokoi et al., J Allergy Clin Immunol., 2008, 121:499-505. Purified mast cells are resuspended in Complete RPMI medium in a sterile 96-well U-bottom plate and incubated in the presence or absence of anti-Siglec-8 antibodies for 30 minutes at concentrations ranging between 0.0001 ng/ml and 10 μg/ml. Samples are incubated for a further 4 to 48 hours with and without purified natural killer (NK) cells or fresh PBL to induce ADCC. Cell-killing by apoptosis or ADCC is analyzed by flow cytometry using fluorescent conjugated antibodies to detect mast cells (CD117 and FcεR1) and Annexin-V and 7AAD to discriminate live and dead or dying cells. Annexin-V and 7AAD staining are performed according to manufacturer's instructions.

In some aspects, an anti-Siglec-8 antibody described herein inhibits mast cell-mediated activities. Mast cell tryptase has been used as a biomarker for total mast cell number and activation. For example, total and active tryptase as well as histamine, N-methyl histamine, and 11-beta-prostaglandin F2 can be measured in blood or urine to assess the reduction in mast cells. See, e.g., U.S. Patent Application Publication No. US 20110293631 for an exemplary mast cell activity assay.

E. Antibody Preparation

The antibody described herein (e.g., an antibody that binds to human Siglec-8) is prepared using techniques available in the art for generating antibodies, exemplary methods of which are described in more detail in the following sections.

1. Antibody Fragments

The present disclosure encompasses antibody fragments. Antibody fragments may be generated by traditional means, such as enzymatic digestion, or by recombinant techniques. In certain circumstances there are advantages of using antibody fragments, rather than whole antibodies. For a review of certain antibody fragments, see Hudson et al. (2003) Nat. Med. 9:129-134.

Various techniques have been developed for the production of antibody fragments. Traditionally, these fragments were derived via proteolytic digestion of intact antibodies (see, e.g., Morimoto et al., Journal of Biochemical and Biophysical Methods 24:107-117 (1992); and Brennan et al., Science, 229:81 (1985)). However, these fragments can now be produced directly by recombinant host cells. Fab, Fv and ScFv antibody fragments can all be expressed in and secreted from E. coli, thus allowing the facile production of large amounts of these fragments. Antibody fragments can be isolated from the antibody phage libraries discussed above. Alternatively, Fab′-SH fragments can be directly recovered from E. coli and chemically coupled to form F(ab′)2 fragments (Carter et al., Bio/Technology 10: 163-167 (1992)). According to another approach, F(ab′)2 fragments can be isolated directly from recombinant host cell culture. Fab and F(ab′)2 fragment with increased in vivo half-life comprising salvage receptor binding epitope residues are described in U.S. Pat. No. 5,869,046. Other techniques for the production of antibody fragments will be apparent to the skilled practitioner. In certain embodiments, an antibody is a single chain Fv fragment (scFv). See WO 93/16185; U.S. Pat. Nos. 5,571,894; and 5,587,458. Fv and scFv are the only species with intact combining sites that are devoid of constant regions; thus, they may be suitable for reduced nonspecific binding during in vivo use. scFv fusion proteins may be constructed to yield fusion of an effector protein at either the amino or the carboxy terminus of an scFv. See Antibody Engineering, ed. Borrebaeck, supra. The antibody fragment may also be a “linear antibody”, e.g., as described in U.S. Pat. No. 5,641,870, for example. Such linear antibodies may be monospecific or bispecific.

2. Humanized Antibodies

The present disclosure encompasses humanized antibodies. Various methods for humanizing non-human antibodies are known in the art. For example, a humanized antibody can have one or more amino acid residues introduced into it from a source which is non-human. These non-human amino acid residues are often referred to as “import” residues, which are typically taken from an “import” variable domain. Humanization can be essentially performed following the method of Winter (Jones et al. (1986) Nature 321:522-525; Riechmann et al. (1988) Nature 332:323-327; Verhoeycn et al. (1988) Science 239:1534-1536), by substituting hypervariable region sequences for the corresponding sequences of a human antibody. Accordingly, such “humanized” antibodies are chimeric antibodies (U.S. Pat. No. 4,816,567) wherein substantially less than an intact human variable domain has been substituted by the corresponding sequence from a non-human species. In practice, humanized antibodies are typically human antibodies in which some hypervariable region residues and possibly some FR residues are substituted by residues from analogous sites in rodent antibodies.

The choice of human variable domains, both light and heavy, to be used in making the humanized antibodies can be important to reduce antigenicity. According to the so-called “best-fit” method, the sequence of the variable domain of a rodent (e.g., mouse) antibody is screened against the entire library of known human variable-domain sequences. The human sequence which is closest to that of the rodent is then accepted as the human framework for the humanized antibody (Sims et al. (1993) J Immunol. 151:2296; Chothia et al. (1987) J. Mol. Biol. 196:901. Another method uses a particular framework derived from the consensus sequence of all human antibodies of a particular subgroup of light or heavy chains. The same framework may be used for several different humanized antibodies (Carter et al. (1992) Proc. Natl. Acad. Sci. USA, 89:4285; Presta et al. (1993) J. Immunol., 151:2623.

It is further generally desirable that antibodies be humanized with retention of high affinity for the antigen and other favorable biological properties. To achieve this goal, according to one method, humanized antibodies are prepared by a process of analysis of the parental sequences and various conceptual humanized products using three-dimensional models of the parental and humanized sequences. Three-dimensional immunoglobulin models are commonly available and are familiar to those, skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the hypervariable region residues are directly and most substantially involved in influencing antigen binding.

3. Human Antibodies

Human anti-Siglec-8 antibodies of the present disclosure can be constructed by combining Fv clone variable domain sequence(s) selected from human-derived phage display libraries with known human constant domain sequences(s). Alternatively, human monoclonal anti-Siglec-8 antibodies of the present disclosure can be made by the hybridoma method. Human myeloma and mouse-human heteromyeloma cell lines for the production of human monoclonal antibodies have been described, for example, by Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991).

It is possible to produce transgenic animals (e.g., mice) that are capable, upon immunization, of producing a full repertoire of human antibodies in the absence of endogenous immunoglobulin production. For example, it has been described that the homozygous deletion of the antibody heavy-chain joining region (JH) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g., Jakobovits et al., Proc. Natl. Acad. Sc. USA, 90: 2551 (1993); Jakobovits et al., Nature, 362: 255 (1993); Bruggermann et al., Year in Immunol., 7: 33 (1993).

Gene shuffling can also be used to derive human antibodies from non-human (e.g., rodent) antibodies, where the human antibody has similar affinities and specificities to the starting non-human antibody. According to this method, which is also called “epitope imprinting”, either the heavy or light chain variable region of a non-human antibody fragment obtained by phage display techniques as described herein is replaced with a repertoire of human V domain genes, creating a population of non-human chain/human chain scFv or Fab chimeras. Selection with antigen results in isolation of a non-human chain/human chain chimeric scFv or Fab wherein the human chain restores the antigen binding site destroyed upon removal of the corresponding non-human chain in the primary phage display clone, i.e., the epitope governs the choice of the human chain partner. When the process is repeated in order to replace the remaining non-human chain, a human antibody is obtained (see PCT WO 93/06213 published Apr. 1, 1993). Unlike traditional humanization of non-human antibodies by CDR grafting, this technique provides completely human antibodies, which have no FR or CDR residues of non-human origin.

4. Bispecific Antibodies

Bispecific antibodies are monoclonal antibodies that have binding specificities for at least two different antigens. In certain embodiments, bispecific antibodies are human or humanized antibodies. In certain embodiments, one of the binding specificities is for Siglec-8 and the other is for any other antigen. In certain embodiments, bispecific antibodies may bind to two different epitopes of Siglec-8. Bispecific antibodies may also be used to localize cytotoxic agents to cells which express Siglec-8. Bispecific antibodies can be prepared as full length antibodies or antibody fragments (e.g. F(ab′)2 bispecific antibodies).

Methods for making bispecific antibodies are known in the art. See Milstein and Cuello, Nature, 305: 537 (1983), WO 93/08829 published May 13, 1993, and Traunecker et al., EMBO J., 10: 3655 (1991). For further details of generating bispecific antibodies see, for example. Suresh et al., Methods in Enzymology, 121:210 (1986). Bispecific antibodies include cross-linked or “heteroconjugate” antibodies. For example, one of the antibodies in the heteroconjugate can be coupled to avidin, the other to biotin. Heteroconjugate antibodies may be made using any convenient cross-linking method. Suitable cross-linking agents are well known in the art, and are disclosed in U.S. Pat. No. 4,676,980, along with a number of cross-linking techniques.

5. Single-Domain Antibodies

In some embodiments, an antibody of the present disclosure is a single-domain antibody. A single-domain antibody is a single polypeptide chain comprising all or a portion of the heavy chain variable domain or all or a portion of the light chain variable domain of an antibody. In certain embodiments, a single-domain antibody is a human single-domain antibody (Domantis, Inc., Waltham, Mass., see, e.g., U.S. Pat. No. 6,248,516 B1). In one embodiment, a single-domain antibody consists of all or a portion of the heavy chain variable domain of an antibody.

6. Antibody Variants

In some embodiments, amino acid sequence modification(s) of the antibodies described herein are contemplated. For example, it may be desirable to improve the binding affinity and/or other biological properties of the antibody. Amino acid sequence variants of the antibody may be prepared by introducing appropriate changes into the nucleotide sequence encoding the antibody, or by peptide synthesis. Such modifications include, for example, deletions from, and/or insertions into and/or substitutions of, residues within the amino acid sequences of the antibody. Any combination of deletion, insertion, and substitution can be made to arrive at the final construct, provided that the final construct possesses the desired characteristics. The amino acid alterations may be introduced in the subject antibody amino acid sequence at the time that sequence is made.

A useful method for identification of certain residues or regions of the antibody that are preferred locations for mutagenesis is called “alanine scanning mutagenesis” as described by Cunningham and Wells (1989) Science, 244:1081-1085. Here, a residue or group of target residues are identified (e.g., charged residues such as arg, asp, his, lys, and glu) and replaced by a neutral or negatively charged amino acid (e.g., alanine or polyalanine) to affect the interaction of the amino acids with antigen. Those amino acid locations demonstrating functional sensitivity to the substitutions then are refined by introducing further or other variants at, or for, the sites of substitution. Thus, while the site for introducing an amino acid sequence variation is predetermined, the nature of the mutation per se need not be predetermined. For example, to analyze the performance of a mutation at a given site, ala scanning or random mutagenesis is conducted at the target codon or region and the expressed immunoglobulins are screened for the desired activity.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme or a polypeptide which increases the serum half-life of the antibody.

In some embodiments, monoclonal antibodies have a C-terminal cleavage at the heavy chain and/or light chain. For example, 1, 2, 3, 4, or 5 amino acid residues are cleaved at the C-terminus of heavy chain and/or light chain. In some embodiments, the C-terminal cleavage removes a C-terminal lysine from the heavy chain. In some embodiments, monoclonal antibodies have an N-terminal cleavage at the heavy chain and/or light chain. For example, 1, 2, 3, 4, or 5 amino acid residues are cleaved at the N-terminus of heavy chain and/or light chain. In some embodiments, truncated forms of monoclonal antibodies can be made by recombinant techniques.

In certain embodiments, an antibody of the present disclosure is altered to increase or decrease the extent to which the antibody is glycosylated. Glycosylation of polypeptides is typically either N-linked or O-linked. N-linked refers to the attachment of a carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used.

Addition or deletion of glycosylation sites to the antibody is conveniently accomplished by altering the amino acid sequence such that one or more of the above-described tripeptide sequences (for N-linked glycosylation sites) is created or removed. The alteration may also be made by the addition, deletion, or substitution of one or more serine or threonine residues to the sequence of the original antibody (for O-linked glycosylation sites).

Where the antibody comprises an Fc region, the carbohydrate attached thereto may be altered. For example, antibodies with a mature carbohydrate structure that lacks fucose attached to an Fc region of the antibody are described in US Pat Appl No US 2003/0157108 (Presta, L.). See also US 2004/0093621 (Kyowa Hakko Kogyo Co., Ltd). Antibodies with a bisecting N-acetylglucosamine (GlcNAc) in the carbohydrate attached to an Fc region of the antibody are referenced in WO 2003/011878, Jean-Mairet et al. and U.S. Pat. No. 6,602,684, Umana et al. Antibodies with at least one galactose residue in the oligosaccharide attached to an Fc region of the antibody are reported in WO 1997/30087, Patel et al. See, also, WO 1998/58964 (Raju, S.) and WO 1999/22764 (Raju, S.) concerning antibodies with altered carbohydrate attached to the Fc region thereof. See also US 2005/0123546 (Umana et al.) on antigen-binding molecules with modified glycosylation.

In certain embodiments, a glycosylation variant comprises an Fc region, wherein a carbohydrate structure attached to the Fc region lacks fucose. Such variants have improved ADCC function. Optionally, the Fc region further comprises one or more amino acid substitutions therein which further improve ADCC, for example, substitutions at positions 298, 333, and/or 334 of the Fc region (Eu numbering of residues). Examples of publications related to “defucosylated” or “fucose-deficient” antibodies include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; Okazaki et al. J. Mol. Biol. 336:1239-1249 (2004); Yamane-Ohnuki et al. Biotech. Bioeng. 87: 614 (2004). Examples of cell lines producing defucosylated antibodies include Lee13 CHO cells deficient in protein fucosylation (Ripka et al. Arch. Biochem. Biophys. 249:533-545 (1986); US Pat Appl No US 2003/0157108 A1, Presta, L; and WO 2004/056312 A1, Adams et al., especially at Example 11), and knockout cell lines, such as alpha-1,6-fucosyltransferase gene, FUT8, knockout CHO cells (Yamane-Ohnuki et al. Biotech. Bioeng. 87: 614 (2004)), and cells overexpressing β1,4-N-acetylglycosminyltransferase III (GnT-III) and Golgi μ-mannosidase II (ManII).

Antibodies are contemplated herein that have reduced fucose relative to the amount of fucose on the same antibody produced in a wild-type CHO cell. For example, the antibody has a lower amount of fucose than it would otherwise have if produced by native CHO cells (e.g., a CHO cell that produce a native glycosylation pattern, such as, a CHO cell containing a native FUT8 gene). In certain embodiments, an anti-Siglec-8 antibody provided herein is one wherein less than about 50%, 40%, 30%, 20%, 10%, 5% or 1% of the N-linked glycans thereon comprise fucose. In certain embodiments, an anti-Siglec-8 antibody provided herein is one wherein none of the N-linked glycans thereon comprise fucose, i.e., wherein the antibody is completely without fucose, or has no fucose or is non-fucosylated or is afucosylated. The amount of fucose can be determined by calculating the average amount of fucose within the sugar chain at Asn297, relative to the sum of all glycostructures attached to Asn297 (e.g., complex, hybrid and high mannose structures) as measured by MALDI-TOF mass spectrometry, as described in WO 2008/077546, for example. Asn297 refers to the asparagine residue located at about position 297 in the Fc region (Eu numbering of Fc region residues); however. Asn297 may also be located about ±3 amino acids upstream or downstream of position 297, i.e., between positions 294 and 300, due to minor sequence variations in antibodies. In some embodiments, at least one or two of the heavy chains of the antibody is non-fucosylated.

In one embodiment, the antibody is altered to improve its serum half-life. To increase the serum half-life of the antibody, one may incorporate a salvage receptor binding epitope into the antibody (especially an antibody fragment) as described in U.S. Pat. No. 5,739,277, for example. As used herein, the term “salvage receptor binding epitope” refers to an epitope of the Fc region of an IgG molecule (e.g., IgG1, IgG2, IgG3, or IgG4) that is responsible for increasing the in vivo scrum half-life of the IgG molecule (US 2003/0190311, U.S. Pat. Nos. 6,821,505; 6,165,745; 5,624,821; 5,648,260; 6,165,745; 5,834,597).

Another type of variant is an amino acid substitution variant. These variants have at least one amino acid residue in the antibody molecule replaced by a different residue. Sites of interest for substitutional mutagenesis include the hypervariable regions, but FR alterations are also contemplated. Conservative substitutions are shown in Table 5 under the heading of “preferred substitutions.” If such substitutions result in a desirable change in biological activity, then more substantial changes, denominated “exemplary substitutions” in Table 5, or as further described below in reference to amino acid classes, may be introduced and the products screened.

TABLE 5

| | | | Preferred |
|---|---|---|---|
| Original Residue | | Exemplary Substitutions | Substitutions |
| Ala | (A) | Val; Leu; Ile | Val |
| Arg | (R) | Lys; Gln; Asn | Lys |
| Asn | (N) | Gln; His; Asp, Lys; Arg | Gln |
| Asp | (D) | Glu; Asn | Glu |
| Cys | (C) | Ser; Ala | Ser |
| Gln | (Q) | Asn; Glu | Asn |
| Glu | (E) | Asp; Gln | Asp |
| Gly | (G) | Ala | Ala |
| His | (H) | Asn; Gln; Lys; Arg | Arg |
| Ile | (I) | Leu; Val; Met; Ala; Phe; Norleucine | Leu |
| Leu | (L) | Norleucine; Ile; Val; Met; Ala; Phe | Ile |
| Lys | (K) | Arg; Gln; Asn | Arg |
| Met | (M) | Leu; Phe; Ile | Leu |
| Phe | (F) | Trp; Leu; Val; Ile; Ala; Tyr | Tyr |
| Pro | (P) | Ala | Ala |
| Ser | (S) | Thr | Thr |
| Thr | (T) | Val; Ser | Ser |
| Trp | (W) | Tyr; Phe | Tyr |
| Tyr | (Y) | Trp; Phe; Thr; Ser | Phe |
| Val | (V) | Ile; Leu; Met; Phe; Ala; Norleucine | Leu |

Substantial modifications in the biological properties of the antibody are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or c) the bulk of the side chain. Amino acids may be grouped according to similarities in the properties of their side chains (in A. L. Lehninger, in Biochemistry, second ed., pp. 73-75, Worth Publishers, New York (1975)):
(1) non-polar: Ala (A), Val (V), Leu (L), Ile (I), Pro (P), Phe (F), Trp (W), Met (M)
(2) uncharged polar: Gly (G), Ser (S), Thr (T), Cys (C), Tyr (Y), Asn (N), Gln (Q)
(3) acidic: Asp (D), Glu (E)
(4) basic: Lys (K), Arg (R), His (H)

Alternatively, naturally occurring residues may be divided into groups based on common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class. Such substituted residues also may be introduced into the conservative substitution sites or, into the remaining (non-conserved) sites.

One type of substitutional variant involves substituting one or more hypervariable region residues of a parent antibody (e.g., a humanized or human antibody). Generally, the resulting variant(s) selected for further development will have modified (e.g., improved) biological properties relative to the parent antibody from which they are generated. A convenient way for generating such substitutional variants involves affinity maturation using phage display. Briefly, several hypervariable region sites (e.g., 6-7 sites) are mutated to generate all possible amino acid substitutions at each site. The antibodies thus generated are displayed from filamentous phage particles as fusions to at least part of a phage coat protein (e.g., the gene III product of M13) packaged within each particle. The phage-displayed variants are then screened for their biological activity (e.g., binding affinity). In order to identify candidate hypervariable region sites for modification, scanning mutagenesis (e.g., alanine scanning) can be performed to identify hypervariable region residues contributing significantly to antigen binding. Alternatively, or additionally, it may be beneficial to analyze a crystal structure of the antigen-antibody complex to identify contact points between the antibody and antigen. Such contact residues and neighboring residues are candidates for substitution according to techniques known in the art, including those elaborated herein. Once such variants are generated, the panel of variants is subjected to screening using techniques known in the art, including those described herein, and antibodies with superior properties in one or more relevant assays may be selected for further development.

Nucleic acid molecules encoding amino acid sequence variants of the antibody are prepared by a variety of methods known in the art. These methods include, but are not limited to, isolation from a natural source (in the case of naturally occurring amino acid sequence variants) or preparation by oligonucleotide-mediated (or site-directed) mutagenesis, PCR mutagenesis, and cassette mutagenesis of an earlier prepared variant or a non-variant version of the antibody.

It may be desirable to introduce one or more amino acid modifications in an Fc region of antibodies of the present disclosure, thereby generating an Fc region variant. The Fc region variant may comprise a human Fc region sequence (e.g., a human IgG1, IgG2, IgG3 or IgG4 Fc region) comprising an amino acid modification (e.g., a substitution) at one or more amino acid positions including that of a hinge cysteine. In some embodiments, the Fc region variant comprises a human IgG4 Fc region. In a further embodiment, the human IgG4 Fc region comprises the amino acid substitution S228P, wherein the amino acid residues are numbered according to the EU index as in Kabat.

In accordance with this description and the teachings of the art, it is contemplated that in some embodiments, an antibody of the present disclosure may comprise one or more alterations as compared to the wild type counterpart antibody, e.g. in the Fc region. These antibodies would nonetheless retain substantially the same characteristics required for therapeutic utility as compared to their wild type counterpart. For example, it is thought that certain alterations can be made in the Fc region that would result in altered (i.e., either improved or diminished) C1q binding and/or Complement Dependent Cytotoxicity (CDC), e.g., as described in WO99/51642. See also Duncan & Winter Nature 322:738-40 (1988); U.S. Pat. Nos. 5,648,260; 5,624,821; and WO94/29351 concerning other examples of Fc region variants. WO00/42072 (Presta) and WO 2004/056312 (Lowman) describe antibody variants with improved or diminished binding to FcRs. The content of these patent publications are specifically incorporated herein by reference. See, also, Shields et al. J. Biol. Chem. 9(2): 6591-6604 (2001). Antibodies with increased half-lives and improved binding to the neonatal Fc receptor (FcRn), which is responsible for the transfer of matemal IgGs to the fetus (Guyer et al., J. Immunol. 117:587 (1976) and Kim et al., J. Immunol. 24:249 (1994)), are described in US2005/0014934A1 (Hinton et al.). These antibodies comprise an Fc region with one or more substitutions therein which improve binding of the Fc region to FcRn. Polypeptide variants with altered Fc region amino acid sequences and increased or decreased C1q binding capability are described in U.S. Pat. No. 6,194,551B1, WO99/51642. The contents of those patent publications are specifically incorporated herein by reference. See, also, Idusogie et al. J. Immunol. 164: 4178-4184 (2000).

7. Vectors, Host Cells, and Recombinant Methods

For recombinant production of an antibody of the present disclosure, the nucleic acid encoding it is isolated and inserted into a replicable vector for further cloning (amplification of the DNA) or for expression. DNA encoding the antibody is readily isolated and sequenced using conventional procedures (e.g., by using oligonucleotide probes that are capable of binding specifically to genes encoding the heavy and light chains of the antibody). Many vectors are available. The choice of vector depends in part on the host cell to be used. Generally, host cells are of either prokaryotic or eukaryotic (generally mammalian) origin. It will be appreciated that constant regions of any isotype can be used for this purpose, including IgG, IgM. IgA, IgD, and IgE constant regions, and that such constant regions can be obtained from any human or animal species.

Generating Antibodies Using Prokaryotic Host Cells:

a) Vector Construction

Polynucleotide sequences encoding polypeptide components of the antibody of the present disclosure can be obtained using standard recombinant techniques. Desired polynucleotide sequences may be isolated and sequenced from antibody producing cells such as hybridoma cells. Alternatively, polynucleotides can be synthesized using nucleotide synthesizer or PCR techniques. Once obtained, sequences encoding the polypeptides are inserted into a recombinant vector capable of replicating and expressing heterologous polynucleotides in prokaryotic hosts. Many vectors that are available and known in the art can be used for the purpose of the present disclosure. Selection of an appropriate vector will depend mainly on the size of the nucleic acids to be inserted into the vector and the particular host cell to be transformed with the vector. Each vector contains various components, depending on its function (amplification or expression of heterologous polynucleotide, or both) and its compatibility with the particular host cell in which it resides. The vector components generally include, but are not limited to: an origin of replication, a selection marker gene, a promoter, a ribosome binding site (RBS), a signal sequence, the heterologous nucleic acid insert and a transcription termination sequence.

In general, plasmid vectors containing replicon and control sequences which are derived from species compatible with the host cell are used in connection with these hosts. The vector ordinarily carries a replication site, as well as marking sequences which are capable of providing phenotypic selection in transformed cells. For example, E. coli is typically transformed using pBR322, a plasmid derived from an E. coli species. pBR322 contains genes-encoding ampicillin (Amp) and tetracycline (Tet) resistance and thus provides easy means for identifying transformed cells. pBR322, its derivatives, or other microbial plasmids or bacteriophage may also contain, or be modified to contain, promoters which can be used by the microbial organism for expression of endogenous proteins. Examples of pBR322 derivatives used for expression of particular antibodies are described in detail in Carter et al., U.S. Pat. No. 5,648,237.

In addition, phage vectors containing replicon and control sequences that are compatible with the host microorganism can be used as transforming vectors in connection with these hosts. For example, bacteriophage such as λGEM.TM.-11 may be utilized in making a recombinant vector which can be used to transform susceptible host cells such as E. coli LE392.

The expression vector of the present disclosure may comprise two or more promoter-cistron pairs, encoding each of the polypeptide components. A promoter is an untranslated regulatory sequence located upstream (5′) to a cistron that modulates its expression. Prokaryotic promoters typically fall into two classes, inducible and constitutive. Inducible promoter is a promoter that initiates increased levels of transcription of the cistron under its control in response to changes in the culture condition, e.g. the presence or absence of a nutrient or a change in temperature.

A large number of promoters recognized by a variety of potential host cells are well known. The selected promoter can be operably linked to cistron DNA encoding the light or heavy chain by removing the promoter from the source DNA via restriction enzyme digestion and inserting the isolated promoter sequence into the vector of the present disclosure. Both the native promoter sequence and many heterologous promoters may be used to direct amplification and/or expression of the target genes. In some embodiments, heterologous promoters are utilized, as they generally permit greater transcription and higher yields of expressed target gene as compared to the native target polypeptide promoter.

Promoters suitable for use with prokaryotic hosts include the PhoA promoter, the β-galactamase and lactose promoter systems, a tryptophan (trp) promoter system and hybrid promoters such as the tac or the trc promoter. However, other promoters that are functional in bacteria (such as other known bacterial or phage promoters) are suitable as well. Their nucleotide sequences have been published, thereby enabling a skilled worker operably to ligate them to cistrons encoding the target light and heavy chains (Siebenlist et al. (1980) Cell 20: 269) using linkers or adaptors to supply any required restriction sites.

In one aspect of the present disclosure, each cistron within the recombinant vector comprises a secretion signal sequence component that directs translocation of the expressed polypeptides across a membrane. In general, the signal sequence may be a component of the vector, or it may be a part of the target polypeptide DNA that is inserted into the vector. The signal sequence selected for the purpose of the present disclosure should be one that is recognized and processed (i.e. cleaved by a signal peptidase) by the host cell. For prokaryotic host cells that do not recognize and process the signal sequences native to the heterologous polypeptides, the signal sequence is substituted by a prokaryotic signal sequence selected, for example, from the group consisting of the alkaline phosphatase, penicillinase, Ipp, or heat-stable enterotoxin II (STII) leaders, LamB, PhoE, PelB, OmpA and MBP. In one embodiment of the present disclosure, the signal sequences used in both cistrons of the expression system are STII signal sequences or variants thereof.

In another aspect, the production of the immunoglobulins according to the present disclosure can occur in the cytoplasm of the host cell, and therefore does not require the presence of secretion signal sequences within each cistron. In that regard, immunoglobulin light and heavy chains are expressed, folded and assembled to form functional immunoglobulins within the cytoplasm. Certain host strains (e.g., the E. coli trxB-strains) provide cytoplasm conditions that are favorable for disulfide bond formation, thereby permitting proper folding and assembly of expressed protein subunits. Proba and Pluckthun Gene, 159:203 (1995).

Antibodies of the present disclosure can also be produced by using an expression system in which the quantitative ratio of expressed polypeptide components can be modulated in order to maximize the yield of secreted and properly assembled antibodies of the present disclosure. Such modulation is accomplished at least in part by simultaneously modulating translational strengths for the polypeptide components.

One technique for modulating translational strength is disclosed in Simmons et al., U.S. Pat. No. 5,840,523. It utilizes variants of the translational initiation region (TIR) within a cistron. For a given TIR, a series of amino acid or nucleic acid sequence variants can be created with a range of translational strengths, thereby providing a convenient means by which to adjust this factor for the desired expression level of the specific chain. TIR variants can be generated by conventional mutagenesis techniques that result in codon changes which can alter the amino acid sequence. In certain embodiments, changes in the nucleotide sequence are silent. Alterations in the TIR can include, for example, alterations in the number or spacing of Shine-Dalgarno sequences, along with alterations in the signal sequence. One method for generating mutant signal sequences is the generation of a “codon bank” at the beginning of a coding sequence that does not change the amino acid sequence of the signal sequence (i.e., the changes are silent). This can be accomplished by changing the third nucleotide position of each codon; additionally, some amino acids, such as leucine, serine, and arginine, have multiple first and second positions that can add complexity in making the bank. This method of mutagenesis is described in detail in Yansura et al. (1992) METHODS: A Companion to Methods in Enzymol. 4:151-158.

In one embodiment, a set of vectors is generated with a range of TIR strengths for each cistron therein. This limited set provides a comparison of expression levels of each chain as well as the yield of the desired antibody products under various TIR strength combinations. TIR strengths can be determined by quantifying the expression level of a reporter gene as described in detail in Simmons et al. U.S. Pat. No. 5,840,523. Based on the translational strength comparison, the desired individual TIRs are selected to be combined in the expression vector constructs of the present disclosure.

Prokaryotic host cells suitable for expressing antibodies of the present disclosure include Archaebacteria and Eubacteria, such as Gram-negative or Gram-positive organisms. Examples of useful bacteria include Escherichia (e.g., E. coli), Bacilli (e.g., B. subtilis), Enterobacteria, Pseudomonas species (e.g., P. aeruginosa), Salmonella typhimurium, Serratia marcescans, Klebsiella, Proteus, Shigella, Rhizobia, Vitreoscilla, or Paracoccus. In one embodiment, gram-negative cells are used. In one embodiment, E. coli cells are used as hosts for the present disclosure. Examples of E. coli strains include strain W3110 (Bachmann, Cellular and Molecular Biology, vol. 2 (Washington, D.C.: American Society for Microbiology, 1987), pp. 1190-1219; ATCC Deposit No. 27,325) and derivatives thereof, including strain 33D3 having genotype W3110 ΔfhuA (ΔtonA) ptr3 lac Iq lacL8 ΔompTΔ(nmpc-fepE) degP41 kanR (U.S. Pat. No. 5,639,635). Other strains and derivatives thereof, such as E. coli 294 (ATCC 31,446), E. coli B, E. coli λ 1776 (ATCC 31,537) and E. coli RV308(ATCC 31,608) are also suitable. These examples are illustrative rather than limiting. Methods for constructing derivatives of any of the above-mentioned bacteria having defined genotypes are known in the art and described in, for example, Bass et al., Proteins, 8:309-314 (1990). It is generally necessary to select the appropriate bacteria taking into consideration replicability of the replicon in the cells of a bacterium. For example, E. coli, Serratia, or Salmonella species can be suitably used as the host when well known plasmids such as pBR322, pBR325, pACYC177, or pKN410 are used to supply the replicon. Typically the host cell should secrete minimal amounts of proteolytic enzymes, and additional protease inhibitors may desirably be incorporated in the cell culture.

b) Antibody Production

Host cells are transformed with the above-described expression vectors and cultured in conventional nutrient media modified as appropriate for inducing promoters, selecting transformants, or amplifying the genes encoding the desired sequences.

Transformation means introducing DNA into the prokaryotic host so that the DNA is replicable, either as an extrachromosomal element or by chromosomal integrant. Depending on the host cell used, transformation is done using standard techniques appropriate to such cells. The calcium treatment employing calcium chloride is generally used for bacterial cells that contain substantial cell-wall barriers. Another method for transformation employs polyethylene glycol/DMSO. Yet another technique used is electroporation.

Prokaryotic cells used to produce the polypeptides of the present disclosure are grown in media known in the art and suitable for culture of the selected host cells. Examples of suitable media include luria broth (LB) plus necessary nutrient supplements. In some embodiments, the media also contains a selection agent, chosen based on the construction of the expression vector, to selectively permit growth of prokaryotic cells containing the expression vector. For example, ampicillin is added to media for growth of cells expressing ampicillin resistant gene.

Any necessary supplements besides carbon, nitrogen, and inorganic phosphate sources may also be included at appropriate concentrations introduced alone or as a mixture with another supplement or medium such as a complex nitrogen source. Optionally the culture medium may contain one or more reducing agents selected from the group consisting of glutathione, cysteine, cystamine, thioglycollate, dithioerythritol and dithiothreitol.

The prokaryotic host cells are cultured at suitable temperatures. In certain embodiments, for E. coli growth, growth temperatures range from about 20° C. to about 39° C.; from about 25° C. to about 37° C.; or about 30° C. The pH of the medium may be any pH ranging from about 5 to about 9, depending mainly on the host organism. In certain embodiments, for E. coli, the pH is from about 6.8 to about 7.4, or about 7.0.

If an inducible promoter is used in the expression vector of the present disclosure, protein expression is induced under conditions suitable for the activation of the promoter. In one aspect of the present disclosure, PhoA promoters are used for controlling transcription of the polypeptides. Accordingly, the transformed host cells are cultured in a phosphate-limiting medium for induction. In certain embodiments, the phosphate-limiting medium is the C.R.A.P. medium (see, e.g., Simmons et al., J. Immunol. Methods (2002), 263:133-147). A variety of other inducers may be used, according to the vector construct employed, as is known in the art.

In one embodiment, the expressed polypeptides of the present disclosure are secreted into and recovered from the periplasm of the host cells. Protein recovery typically involves disrupting the microorganism, generally by such means as osmotic shock, sonication or lysis. Once cells are disrupted, cell debris or whole cells may be removed by centrifugation or filtration. The proteins may be further purified, for example, by affinity resin chromatography. Alternatively, proteins can be transported into the culture media and isolated therein. Cells may be removed from the culture and the culture supernatant being filtered and concentrated for further purification of the proteins produced. The expressed polypeptides can be further isolated and identified using commonly known methods such as polyacrylamide gel electrophoresis (PAGE) and Western blot assay.

In one aspect of the present disclosure, antibody production is conducted in large quantity by a fermentation process. Various large-scale fed-batch fermentation procedures are available for production of recombinant proteins. Large-scale fermentations have at least 1000 liters of capacity, and in certain embodiments, about 1,000 to 100,000 liters of capacity. These fermentors use agitator impellers to distribute oxygen and nutrients, especially glucose. Small scale fermentation refers generally to fermentation in a fermentor that is no more than approximately 100 liters in volumetric capacity, and can range from about 1 liter to about 100 liters.

In a fermentation process, induction of protein expression is typically initiated after the cells have been grown under suitable conditions to a desired density. e.g., an OD550 of about 180-220, at which stage the cells are in the early stationary phase. A variety of inducers may be used, according to the vector construct employed, as is known in the art and described above. Cells may be grown for shorter periods prior to induction. Cells are usually induced for about 12-50 hours, although longer or shorter induction time may be used.

To improve the production yield and quality of the polypeptides of the present disclosure, various fermentation conditions can be modified. For example, to improve the proper assembly and folding of the secreted antibody polypeptides, additional vectors overexpressing chaperone proteins, such as Dsb proteins (DsbA, DsbB, DsbC, DsbD and or DsbG) or FkpA (a peptidylprolyl cis,trans-isomerase with chaperone activity) can be used to co-transform the host prokaryotic cells. The chaperone proteins have been demonstrated to facilitate the proper folding and solubility of heterologous proteins produced in bacterial host cells. Chen et al. (1999) J. Biol. Chem. 274:19601-19605; Georgiou et al., U.S. Pat. No. 6,083,715; Georgiou et al., U.S. Pat. No. 6,027,888; Bothmann and Pluckthun (2000) J. Biol. Chem. 275:17100-17105; Ramm and Pluckthun (2000) J. Biol. Chem. 275:17106-17113; Arie et al. (2001) Mol. Microbiol. 39:199-210.

To minimize proteolysis of expressed heterologous proteins (especially those that are proteolytically sensitive), certain host strains deficient for proteolytic enzymes can be used for the present disclosure. For example, host cell strains may be modified to effect genetic mutation(s) in the genes encoding known bacterial proteases such as Protease III, OmpT, DegP, Tsp, Protease I, Protease Mi, Protease V, Protease VI and combinations thereof. Some E. coli protease-deficient strains are available and described in, for example, Joly et al. (1998), supra; Georgiou et al., U.S. Pat. No. 5,264,365; Georgiou et al., U.S. Pat. No. 5,508,192: Hara et al., Microbial Drug Resistance, 2:63-72 (1996).

In one embodiment, E. coli strains deficient for proteolytic enzymes and transformed with plasmids overexpressing one or more chaperone proteins are used as host cells in the expression system of the present disclosure.

c) Antibody Purification

In one embodiment, the antibody protein produced herein is further purified to obtain preparations that are substantially homogeneous for further assays and uses. Standard protein purification methods known in the art can be employed. The following procedures are exemplary of suitable purification procedures: fractionation on immunoaffinity or ion-exchange columns, ethanol precipitation, reverse phase HPLC, chromatography on silica or on a cation-exchange resin such as DEAE, chromatofocusing, SDS-PAGE, ammonium sulfate precipitation, and gel filtration using, for example, Sephadex G-75.

In one aspect, Protein A immobilized on a solid phase is used for immunoaffinity purification of the antibody products of the present disclosure. Protein A is a 41 kD cell wall protein from Staphylococcus aureus which binds with a high affinity to the Fc region of antibodies. Lindmark et al (1983) J. Immunol. Meth. 62:1-13. The solid phase to which Protein A is immobilized can be a column comprising a glass or silica surface, or a controlled pore glass column or a silicic acid column. In some applications, the column is coated with a reagent, such as glycerol, to possibly prevent nonspecific adherence of contaminants.

As the first step of purification, a preparation derived from the cell culture as described above can be applied onto a Protein A immobilized solid phase to allow specific binding of the antibody of interest to Protein A. The solid phase would then be washed to remove contaminants non-specifically bound to the solid phase. Finally the antibody of interest is recovered from the solid phase by elution.

Generating Antibodies Using Eukaryotic Host Cells:

A vector for use in a eukaryotic host cell generally includes one or more of the following non-limiting components: a signal sequence, an origin of replication, one or more marker genes, an enhancer element, a promoter, and a transcription termination sequence.

a) Signal Sequence Component

A vector for use in a eukaryotic host cell may also contain a signal sequence or other polypeptide having a specific cleavage site at the N-terminus of the mature protein or polypeptide of interest. The heterologous signal sequence selected may be one that is recognized and processed (i.e., cleaved by a signal peptidase) by the host cell. In mammalian cell expression, mammalian signal sequences as well as viral secretory leaders, for example, the herpes simplex gD signal, are available. The DNA for such a precursor region is ligated in reading frame to DNA encoding the antibody.

b) Origin of Replication

Generally, an origin of replication component is not needed for mammalian expression vectors. For example, the SV40 origin may typically be used only because it contains the early promoter.

c) Selection Gene Component

Expression and cloning vectors may contain a selection gene, also termed a selectable marker. Typical selection genes encode proteins that (a) confer resistance to antibiotics or other toxins, e.g., ampicillin, neomycin, methotrexate, or tetracycline. (b) complement auxotrophic deficiencies, where relevant, or (c) supply critical nutrients not available from complex media.

One example of a selection scheme utilizes a drug to arrest growth of a host cell. Those cells that are successfully transformed with a heterologous gene produce a protein conferring drug resistance and thus survive the selection regimen. Examples of such dominant selection use the drugs neomycin, mycophenolic acid and hygromycin.

Another example of suitable selectable markers for mammalian cells are those that enable the identification of cells competent to take up the antibody nucleic acid, such as DHFR, thymidine kinase, metallothionein-I and -II, primate metallothionein genes, adenosine deaminase, omithine decarboxylase, etc.

For example, in some embodiments, cells transformed with the DHFR selection gene are first identified by culturing all of the transformants in a culture medium that contains methotrexate (Mtx), a competitive antagonist of DHFR. In some embodiments, an appropriate host cell when wild-type DHFR is employed is the Chinese hamster ovary (CHO) cell line deficient in DHFR activity (e.g., ATCC CRL-9096).

Alternatively, host cells (particularly wild-type hosts that contain endogenous DHFR) transformed or co-transformed with DNA sequences encoding an antibody, wild-type DHFR protein, and another selectable marker such as aminoglycoside 3′-phosphotransferase (APH) can be selected by cell growth in medium containing a selection agent for the selectable marker such as an aminoglycosidic antibiotic, e.g., kanamycin, neomycin, or G418. See U.S. Pat. No. 4,965,199. Host cells may include NS0, CHOK1, CHOK1SV or derivatives, including cell lines deficient in glutamine synthetase (GS). Methods for the use of GS as a selectable marker for mammalian cells are described in U.S. Pat. Nos. 5,122,464 and 5,891,693.

d) Promoter Component

Expression and cloning vectors usually contain a promoter that is recognized by the host organism and is operably linked to nucleic acid encoding a polypeptide of interest (e.g., an antibody). Promoter sequences are known for eukaryotes. For example, virtually all eukaryotic genes have an AT-rich region located approximately 25 to 30 bases upstream from the site where transcription is initiated. Another sequence found 70 to 80 bases upstream from the start of transcription of many genes is a CNCAAT region where N may be any nucleotide. At the 3′ end of most eukaryotic genes is an AATAAA sequence that may be the signal for addition of the poly A tail to the 3′ end of the coding sequence. In certain embodiments, any or all of these sequences may be suitably inserted into eukaryotic expression vectors.

Transcription from vectors in mammalian host cells is controlled, for example, by promoters obtained from the genomes of viruses such as polyoma virus, fowlpox virus, adenovirus (such as Adenovirus 2), bovine papilloma virus, avian sarcoma virus, cytomegalovirus, a retrovirus, hepatitis-B virus and Simian Virus 40 (SV40), from heterologous mammalian promoters. e.g., the actin promoter or an immunoglobulin promoter, from heat-shock promoters, provided such promoters are compatible with the host cell systems.

The early and late promoters of the SV40 virus are conveniently obtained as an SV40 restriction fragment that also contains the SV40 viral origin of replication. The immediate early promoter of the human cytomegalovirus is conveniently obtained as a HindIII E restriction fragment. A system for expressing DNA in mammalian hosts using the bovine papilloma virus as a vector is disclosed in U.S. Pat. No. 4,419,446. A modification of this system is described in U.S. Pat. No. 4,601,978. See also Reyes et al., Nature 297:598-601 (1982), describing expression of human β-interferon cDNA in mouse cells under the control of a thymidine kinase promoter from herpes simplex virus. Alternatively, the Rous Sarcoma Virus long terminal repeat can be used as the promoter.

e) Enhancer Element Component

Transcription of DNA encoding an antibody of the present disclosure by higher eukaryotes is often increased by inserting an enhancer sequence into the vector. Many enhancer sequences are now known from mammalian genes (globin, elastase, albumin, α-fetoprotein, and insulin). Typically, however, one will use an enhancer from a eukaryotic cell virus. Examples include the SV40 enhancer on the late side of the replication origin (bp 100-270), the human cytomegalovirus early promoter enhancer, the mouse cytomegalovirus early promoter enhancer, the polyoma enhancer on the late side of the replication origin, and adenovirus enhancers. See also Yaniv, Nature 297:17-18 (1982) describing enhancer elements for activation of eukaryotic promoters. The enhancer may be spliced into the vector at a position 5′ or 3′ to the antibody polypeptide-encoding sequence, but is generally located at a site 5′ from the promoter.

f) Transcription Termination Component

Expression vectors used in eukaryotic host cells may also contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of the mRNA encoding an antibody. One useful transcription termination component is the bovine growth hormone polyadenylation region. See WO94/11026 and the expression vector disclosed therein.

g) Selection and Transformation of Host Cells

Suitable host cells for cloning or expressing the DNA in the vectors herein include higher eukaryote cells described herein, including vertebrate host cells. Propagation of vertebrate cells in culture (tissue culture) has become a routine procedure. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/-DHFR (CHO, Urlaub et al., Proc. Natl. Acad. Sci. USA 77:4216 (1980)); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2), canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51): TRI cells (Mather et al., Annals N.Y. Acad. Sci. 383:44-68 (1982)); MRC 5 cells; FS4 cells; CHOK1 cells, CHOKISV cells or derivatives and a human hepatoma line (Hep G2).

Host cells are transformed with the above-described-expression or cloning vectors for antibody production and cultured in conventional nutrient media modified as appropriate for inducing promoters, selecting transformants, or amplifying the genes encoding the desired sequences.

h) Culturing the Host Cells

The host cells used to produce an antibody of the present disclosure may be cultured in a variety of media. Commercially available media such as Ham's F10 (Sigma), Minimal Essential Medium ((MEM), Sigma), RPMI-1640 (Sigma), and Dulbecco's Modified Eagle's Medium ((DMEM), Sigma) are suitable for culturing the host cells. In addition, any of the media described in Ham et al., Meth. Enz. 58:44 (1979), Barnes et al., Anal. Biochem. 102:255 (1980), U.S. Pat. No. 4,767,704; 4,657,866; 4,927,762; 4,560,655; or 5,122,469; WO 90/03430; WO 87/00195; or U.S. Pat. Re. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™ drug), trace elements (defined as inorganic compounds usually present at final concentrations in the micromolar range), and glucose or an equivalent energy source. Any other supplements may also be included at appropriate concentrations that would be known to those skilled in the art. The culture conditions, such as temperature, pH, and the like, are those previously used with the host cell selected for expression, and will be apparent to the ordinarily skilled artisan.

i) Purification of Antibody

When using recombinant techniques, the antibody can be produced intracellularly, or directly secreted into the medium. If the antibody is produced intracellularly, as a first step, the particulate debris, either host cells or lysed fragments, may be removed, for example, by centrifugation or ultrafiltration. Where the antibody is secreted into the medium, supernatants from such expression systems may be first concentrated using a commercially available protein concentration filter, for example, an Amicon or Millipore Pellicon ultrafiltration unit. A protease inhibitor such as PMSF may be included in any of the foregoing steps to inhibit proteolysis, and antibiotics may be included to prevent the growth of adventitious contaminants.

The antibody composition prepared from the cells can be purified using, for example, hydroxylapatite chromatography, gel electrophoresis, dialysis, and affinity chromatography, with affinity chromatography being a convenient technique. The suitability of protein A as an affinity ligand depends on the species and isotype of any immunoglobulin Fc domain that is present in the antibody. Protein A can be used to purify antibodies that are based on human γ1, γ2, or γ4 heavy chains (Lindmark et al., J. Immunol. Methods 62:1-13 (1983)). Protein G is recommended for all mouse isotypes and for human γ3 (Guss et al., EMBO J. 5:15671575 (1986)). The matrix to which the affinity ligand is attached may be agarose, but other matrices are available. Mechanically stable matrices such as controlled pore glass or poly(styrenedivinyl)benzene allow for faster flow rates and shorter processing times than can be achieved with agarose. Where the antibody comprises a CH3 domain, the Bakerbond ABX™ resin (J. T. Baker. Phillipsburg, N.J.) is useful for purification. Other techniques for protein purification such as fractionation on an ion-exchange column, ethanol precipitation, Reverse Phase HPLC, chromatography on silica, chromatography on heparin SEPHAROSE™ chromatography on an anion or cation exchange resin (such as a polyaspartic acid column), chromatofocusing, SDS-PAGE, and ammonium sulfate precipitation are also available depending on the antibody to be recovered.

Following any preliminary purification step(s), the mixture comprising the antibody of interest and contaminants may be subjected to further purification, for example, by low pH hydrophobic interaction chromatography using an elution buffer at a pH between about 2.5-4.5, performed at low salt concentrations (e.g., from about 0-0.25M salt).

In general, various methodologies for preparing antibodies for use in research, testing, and clinical use are well-established in the art, consistent with the above-described methodologies and/or as deemed appropriate by one skilled in the art for a particular antibody of interest.

Production of Non-Fucosylated Antibodies

Provided herein are methods for preparing antibodies with a reduced degree of fucosylation. For example, methods contemplated herein include, but are not limited to, use of cell lines deficient in protein fucosylation (e.g., Lee13 CHO cells, alpha-1,6-fucosyltransferase gene knockout CHO cells, cells overexpressing β1,4-N-acetylglycosminyltransferase III and further overexpressing Golgi μ-mannosidase II, etc.), and addition of a fucose analog(s) in a cell culture medium used for the production of the antibodies. See Ripka et al. Arch. Biochem. Biophys. 249:533-545 (1986); US Pat Appl No US 2003/0157108 A1, Presta, L; WO 2004/056312 A1; Yamane-Ohnuki et al. Biotech. Bioeng. 87: 614 (2004); and U.S. Pat. No. 8,574,907. Additional techniques for reducing the fucose content of antibodies include Glymaxx technology described in U.S. Patent Application Publication No. 2012/0214975. Additional techniques for reducing the fucose content of antibodies also include the addition of one or more glycosidase inhibitors in a cell culture medium used for the production of the antibodies. Glycosidase inhibitors include α-glucosidase I, α-glucosidase II, and α-mannosidase I. In some embodiments, the glycosidase inhibitor is an inhibitor of α-mannosidase I (e.g., kifunensine).

As used herein, “core fucosylation” refers to addition of fucose (“fucosylation”) to N-acetylglucosamine (“GlcNAc”) at the reducing terminal of an N-linked glycan. Also provided are antibodies produced by such methods and compositions thereof.

In some embodiments, fucosylation of complex N-glycoside-linked sugar chains bound to the Fc region (or domain) is reduced. As used herein, a “complex N-glycoside-linked sugar chain” is typically bound to asparagine 297 (according to the number of Kabat), although a complex N-glycoside linked sugar chain can also be linked to other asparagine residues. A “complex N-glycoside-linked sugar chain” excludes a high mannose type of sugar chain, in which only mannose is incorporated at the non-reducing terminal of the core structure, but includes 1) a complex type, in which the non-reducing terminal side of the core structure has one or more branches of galactose-N-acetylglucosamine (also referred to as “gal-GlcNAc”) and the non-reducing terminal side of Gal-GlcNAc optionally has a sialic acid, bisecting N-acetylglucosamine or the like; or 2) a hybrid type, in which the non-reducing terminal side of the core structure has both branches of the high mannose N-glycoside-linked sugar chain and complex N-glycoside-linked sugar chain.

In some embodiments, the “complex N-glycoside-linked sugar chain” includes a complex type in which the non-reducing terminal side of the core structure has zero, one or more branches of galactose-N-acetylglucosamine (also referred to as “gal-GlcNAc”) and the non-reducing terminal side of Gal-GlcNAc optionally further has a structure such as a sialic acid, bisecting N-acetylglucosamine or the like.

According to the present methods, typically only a minor amount of fucose is incorporated into the complex N-glycoside-linked sugar chain(s). For example, in various embodiments, less than about 60%, less than about 50%, less than about 40%, less than about 30%, less than about 20%, less than about 15%, less than about 10%, less than about 5%, or less than about 1% of the antibody has core fucosylation by fucose in a composition. In some embodiments, substantially none (i.e., less than about 0.5%) of the antibody has core fucosylation by fucose in a composition. In some embodiments, more than about 40%, more than about 50%, more than about 60%, more than about 70%, more than about 80%, more than about 90%, more than about 91%, more than about 92%, more than about 93%, more than about 94%, more than about 95%, more than about 96%, more than about 97%, more than about 98%, or more than about 99% of the antibody is nonfucosylated in a composition.

In some embodiments, provided herein is an antibody wherein substantially none (i.e., less than about 0.5%) of the N-glycoside-linked carbohydrate chains contain a fucose residue. In some embodiments, provided herein is an antibody wherein at least one or two of the heavy chains of the antibody is non-fucosylated.

As described above, a variety of mammalian host-expression vector systems can be utilized to express an antibody. In some embodiments, the culture media is not supplemented with fucose. In some embodiments, an effective amount of a fucose analog is added to the culture media. In this context, an “effective amount” refers to an amount of the analog that is sufficient to decrease fucose incorporation into a complex N-glycoside-linked sugar chain of an antibody by at least about 10%, at least about 20%, at least about 30%, at least about 40% or at least about 50%. In some embodiments, antibodies produced by the instant methods comprise at least about 10%, at least about 20%, at least about 30%, at least about 40% or at least about 50% non-core fucosylated protein (e.g., lacking core fucosylation), as compared with antibodies produced from the host cells cultured in the absence of a fucose analog.

The content (e.g., the ratio) of sugar chains in which fucose is not bound to N-acetylglucosamine in the reducing end of the sugar chain versus sugar chains in which fucose is bound to N-acetylglucosamine in the reducing end of the sugar chain can be determined, for example, as described in the Examples. Other methods include hydrazinolysis or enzyme digestion (see, e.g., Biochemical Experimentation Methods 23: Method for Studying Glycoprotein Sugar Chain (Japan Scientific Societies Press), edited by Reiko Takahashi (1989)), fluorescence labeling or radioisotope labeling of the released sugar chain and then separating the labeled sugar chain by chromatography. Also, the compositions of the released sugar chains can be determined by analyzing the chains by the HPAEC-PAD method (see, e.g., J. Liq Chromatogr. 6:1557 (1983)). (See generally U.S. Patent Application Publication No. 2004/0110282.).

### III. Compositions

In some aspects, also provided herein are compositions (e.g., pharmaceutical compositions) comprising any of the anti-Siglec-8 antibodies described herein (e.g., an antibody that binds to Siglec-8). In some aspects, provided herein is a composition comprising an anti-Siglec-8 antibody described herein, wherein the antibody comprises a Fc region and N-glycoside-linked carbohydrate chains linked to the Fc region, wherein less than about 50% of the N-glycoside-linked carbohydrate chains contain a fucose residue. In some embodiments, the antibody comprises a Fc region and N-glycoside-linked carbohydrate chains linked to the Fc region, wherein less than about 45%, about 40%, about 35%, about 30%, about 25%, about 20%, or about 15% of the N-glycoside-linked carbohydrate chains contain a fucose residue. In some aspects, provided herein is a composition comprising an anti-Siglec-8 antibody described herein, wherein the antibody comprises a Fc region and N-glycoside-linked carbohydrate chains linked to the Fc region, wherein substantially none of the N-glycoside-linked carbohydrate chains contain a fucose residue.

Therapeutic formulations are prepared for storage by mixing the active ingredient having the desired degree of purity with optional pharmaceutically acceptable carriers, excipients or stabilizers (Remington: The Science and Practice of Pharmacy, 20th Ed., Lippincott Williams & Wiklins, Pub., Gennaro Ed., Philadelphia, Pa. 2000). Acceptable carriers, excipients, or stabilizers are nontoxic to recipients at the dosages and concentrations employed, and include buffers, antioxidants including ascorbic acid, methionine, Vitamin E, sodium metabisulfite; preservatives, isotonicifiers, stabilizers, metal complexes (e.g., Zn-protein complexes); chelating agents such as EDTA and/or non-ionic surfactants.

Buffers can be used to control the pH in a range which optimizes the therapeutic effectiveness, especially if stability is pH dependent. Buffers can be present at concentrations ranging from about 50 mM to about 250 mM. Suitable buffering agents for use with the present disclosure include both organic and inorganic acids and salts thereof. For example, citrate, phosphate, succinate, tartrate, fumarate, gluconate, oxalate, lactate, acetate. Additionally, buffers may be comprised of histidine and trimethylamine salts such as Tris.

Preservatives can be added to prevent microbial growth, and are typically present in a range from about 0.2%-1.0% (w/v). Suitable preservatives for use with the present disclosure include octadecyldimethylbenzyl ammonium chloride; hexamethonium chloride; benzalkonium halides (e.g., chloride, bromide, iodide), benzethonium chloride; thimerosal, phenol, butyl or benzyl alcohol; alkyl parabens such as methyl or propyl paraben; catechol; resorcinol; cyclohexanol, 3-pentanol, and m-cresol.

Tonicity agents, sometimes known as “stabilizers” can be present to adjust or maintain the tonicity of liquid in a composition. When used with large, charged biomolecules such as proteins and antibodies, they are often termed “stabilizers” because they can interact with the charged groups of the amino acid side chains, thereby lessening the potential for inter and intra-molecular interactions. Tonicity agents can be present in any amount between about 0.1% to about 25% by weight or between about 1 to about 5% by weight, taking into account the relative amounts of the other ingredients. In some embodiments, tonicity agents include polyhydric sugar alcohols, trihydric or higher sugar alcohols, such as glycerin, erythritol, arabitol, xylitol, sorbitol and mannitol.

Additional excipients include agents which can serve as one or more of the following: (1) bulking agents, (2) solubility enhancers, (3) stabilizers and (4) and agents preventing denaturation or adherence to the container wall. Such excipients include: polyhydric sugar alcohols (enumerated above); amino acids such as alanine, glycine, glutamine, asparagine, histidine, arginine, lysine, ornithine, leucine, 2-phenylalanine, glutamic acid, threonine, etc.; organic sugars or sugar alcohols such as sucrose, lactose, lactitol, trehalose, stachyose, mannose, sorbose, xylose, ribose, ribitol, myoinisitose, myoinisitol, galactose, galactitol, glycerol, cyclitols (e.g., inositol), polyethylene glycol; sulfur containing reducing agents, such as urea, glutathione, thioctic acid, sodium thioglycolate, thioglycerol, α-monothioglycerol and sodium thio sulfate; low molecular weight proteins such as human serum albumin, bovine serum albumin, gelatin or other immunoglobulins; hydrophilic polymers such as polyvinylpyrrolidone; monosaccharides (e.g., xylose, mannose, fructose, glucose; disaccharides (e.g., lactose, maltose, sucrose); trisaccharides such as raffinose; and polysaccharides such as dextrin or dextran.

Non-ionic surfactants or detergents (also known as “wetting agents”) can be present to help solubilize the therapeutic agent as well as to protect the therapeutic protein against agitation-induced aggregation, which also permits the formulation to be exposed to shear surface stress without causing denaturation of the active therapeutic protein or antibody. Non-ionic surfactants are present in a range of about 0.05 mg/ml to about 1.0 mg/ml or about 0.07 mg/ml to about 0.2 mg/ml. In some embodiments, non-ionic surfactants are present in a range of about 0.001% to about 0.1% w/v or about 0.01% to about 0.1% w/v or about 0.01% to about 0.025% w/v.

Suitable non-ionic surfactants include polysorbates (20, 40, 60, 65, 80, etc.), poloxamers (184, 188, etc.), PLURONIC® polyols, TRITONV, polyoxyethylene sorbitan monoethers (TWEEN®-20, TWEEN®-80, etc.), lauromacrogol 400, polyoxyl 40 stearate, polyoxyethylene hydrogenated castor oil 10, 50 and 60, glycerol monostearate, sucrose fatty acid ester, methyl celluose and carboxymethyl cellulose. Anionic detergents that can be used include sodium lauryl sulfate, dioctyle sodium sulfosuccinate and dioctyl sodium sulfonate. Cationic detergents include benzalkonium chloride or benzethonium chloride.

In order for the formulations to be used for in vivo administration, they must be sterile. The formulation may be rendered sterile by filtration through sterile filtration membranes. The therapeutic compositions herein generally are placed into a container having a sterile access port, for example, an intravenous solution bag or vial having a stopper pierceable by a hypodermic injection needle.

The route of administration is in accordance with known and accepted methods, such as by single or multiple bolus or infusion over a long period of time in a suitable manner, e.g., injection or infusion by subcutaneous, intravenous, intraperitoneal, intramuscular, intraarterial, intralesional or intraarticular routes, topical administration, inhalation or by sustained release or extended-release means. In some embodiments, a composition or anti-Siglec-8 antibody of the present disclosure is administered by intravenous infusion once a month for 3 or more months. In some embodiments, a composition or anti-Siglec-8 antibody of the present disclosure is administered by intravenous infusion once per cycle (e.g., on Day 1) for 1, 2, 3, 4, 5, or 6 cycles, wherein each cycle is 1 month, 4 weeks, or 28 days.

The formulation herein may also contain more than one active compound as necessary for the particular indication being treated, preferably those with complementary activities that do not adversely affect each other. Such active compounds are suitably present in combination in amounts that are effective for the purpose intended.

### IV. Articles of Manufacture or Kits

In another aspect, an article of manufacture or kit is provided which comprises an anti-Siglec-8 antibody described herein (e.g., an antibody that binds human Siglec-8). The article of manufacture or kit may further comprise instructions for use of the antibody in the methods of the present disclosure. Thus, in certain embodiments, the article of manufacture or kit comprises instructions for the use of an anti-Siglec-8 antibody that binds to human Siglec-8 in methods for treating and/or preventing mast cell gastritis, mast cell esophagitis, mast cell colitis, mast cell enteritis, mast cell duodenitis, and/or mast cell gastroenteritis in an individual comprising administering to the individual an effective amount of an anti-Siglec-8 antibody that binds to human Siglec-8. In certain embodiments, the article of manufacture comprises a medicament comprising an antibody that binds to human Siglec-8 and a package insert comprising instructions for administration of the medicament in an individual in need thereof to treat and/or prevent mast cell gastritis, mast cell esophagitis, mast cell colitis, mast cell enteritis, mast cell duodenitis, and/or mast cell gastroenteritis. In some embodiments, the package insert further indicates that the treatment is effective in reducing one or more symptoms in the individual with mast cell gastritis, mast cell esophagitis, mast cell colitis, mast cell enteritis, mast cell duodenitis, and/or mast cell gastroenteritis as compared to a baseline level before administration of the medicament. In some embodiments, the individual is diagnosed with mast cell gastritis, mast cell colitis, mast cell duodenitis, mast cell enteritis, or mast cell gastroenteritis before administration of the medicament comprising the antibody. In certain embodiments, the individual is a human.

The article of manufacture or kit may further comprise a container. Suitable containers include, for example, bottles, vials (e.g., dual chamber vials), syringes (such as single or dual chamber syringes) and test tubes. The container may be formed from a variety of materials such as glass or plastic. The container holds the formulation.

The article of manufacture or kit may further comprise a label or a package insert, which is on or associated with the container, may indicate directions for reconstitution and/or use of the formulation. The label or package insert may further indicate that the formulation is useful or intended for subcutaneous, intravenous, or other modes of administration for treating and/or preventing mast cell gastritis, mast cell esophagitis, mast cell colitis, mast cell enteritis, mast cell duodenitis, and/or mast cell gastroenteritis in an individual. The container holding the formulation may be a single-use vial or a multi-use vial, which allows for repeat administrations of the reconstituted formulation. The article of manufacture or kit may further comprise a second container comprising a suitable diluent. The article of manufacture or kit may further include other materials desirable from a commercial, therapeutic, and user standpoint, including other buffers, diluents, filters, needles, syringes, and package inserts with instructions for use.

In a specific embodiment, the present disclosure provides kits for a single dose-administration unit. Such kits comprise a container of an aqueous formulation of therapeutic antibody, including both single or multi-chambered pre-filled syringes. Exemplary pre-filled syringes are available from Vetter GmbH, Ravensburg, Germany.

In another embodiment, provided herein is an article of manufacture or kit comprising the formulations described herein for administration in an auto-injector device. An auto-injector can be described as an injection device that upon activation, will deliver its contents without additional necessary action from the patient or administrator. They are particularly suited for self-medication of therapeutic formulations when the delivery rate must be constant and the time of delivery is greater than a few moments.

In another aspect, an article of manufacture or kit is provided which comprises an anti-Siglec-8 antibody described herein (e.g., an antibody that binds human Siglec-8). The article of manufacture or kit may further comprise instructions for use of the antibody in the methods of the present disclosure. Thus, in certain embodiments, the article of manufacture or kit comprises instructions for the use of an anti-Siglec-8 antibody that binds to human Siglec-8 in methods for treating or preventing mast cell gastritis, mast cell esophagitis, mast cell colitis, mast cell enteritis, mast cell duodenitis, and/or mast cell gastroenteritis in an individual comprising administering to the individual an effective amount of an anti-Siglec-8 antibody that binds to human Siglec-8. In certain embodiments, the article of manufacture or kit comprises a medicament comprising an antibody that binds to human Siglec-8 and a package insert comprising instructions for administration of the medicament in an individual in need thereof to treat and/or prevent mast cell gastritis, mast cell esophagitis, mast cell colitis, mast cell enteritis, mast cell duodenitis, and/or mast cell gastroenteritis.

The present disclosure also provides an article of manufacture or kit which comprises an anti-Siglec-8 antibody described herein (e.g., an antibody that binds human Siglec-8) in combination with one or more additional medicament (e.g., a second medicament) for treating or preventing mast cell gastritis, mast cell esophagitis, mast cell colitis, mast cell enteritis, mast cell duodenitis, and/or mast cell gastroenteritis in an individual. The article of manufacture or kit may further comprise instructions for use of the antibody in combination with one or more additional medicament in the methods of the present disclosure. For example, the article of manufacture or kit herein optionally further comprises a container comprising a second medicament, wherein the anti-Siglec-8 antibody is a first medicament, and which article or kit further comprises instructions on the label or package insert for treating the individual with the second medicament, in an effective amount. Thus in certain embodiments, the article of manufacture or kit comprises instructions for the use of an anti-Siglec-8 antibody that binds to human Siglec-8 in combination with one or more additional medicament in methods for treating or preventing mast cell gastritis, mast cell esophagitis, mast cell colitis, mast cell enteritis, mast cell duodenitis, and/or mast cell gastroenteritis in an individual. In certain embodiments, the article of manufacture or kit comprises a medicament comprising an antibody that binds to human Siglec-8 (e.g., a first medicament), one or more additional medicament and a package insert comprising instructions for administration of the first medicament in combination with the one or more additional medicament (e.g., a second medicament). In some embodiments, the one or more additional therapeutic agents may include, but are not limited to, PPIs, systemic corticosteroids, topical corticosteroids, antihistamines, mast cell stabilizers, H-2 blockers, anti-IgE antibodies, calcineurin inhibitors, immunomodulatory agents, and immunosuppressive agents (e.g., azathioprine, 6-MP, MMF, and mTOR inhibitors).

It is understood that the aspects and embodiments described herein are for illustrative purposes only and that various modifications or changes in light thereof will be suggested to persons skilled in the art and are to be included within the spirit and purview of this application and scope of the appended claims.

### EXAMPLES

The present disclosure will be more fully understood by reference to the following examples. The examples should not, however, be construed as limiting the scope of the present disclosure. It is understood that the examples and embodiments described herein are for illustrative purposes only and that various modifications or changes in light thereof will be suggested to persons skilled in the art and are to be included within the spirit and purview of this application and scope of the appended claims.

### Example 1: Structure of a Phase 1b, Open-Label, Dose-Escalating, Proof-of-Concept Study to Assess the Safety, Tolerability, and Clinical Benefit of Anti-Siglec-8 Antibody Treatment in Patients with Mast Cell Gastritis and/or Gastroenteritis

An ongoing study evaluating the efficacy and safety of anti-Siglec-8 antibody for treatment of patients with eosinophilic gastritis and/or gastroenteritis found a subpopulation of patients that, despite meeting the symptom criteria of abdominal pain, nausea and/or diarrhea, did not have the pre-requisite number of eosinophils in the gastric and/or duodenal mucosa. Instead, it was found that these patients had a substantial number of mast cells (in most cases greater than 30 mast cells/high power field (HPF)) in the stomach and/or duodenal mucosa. Normal levels have been measured to be approximately less than 20 mast cells/HPF (Doyle et al., Am. J. Surg. Pathol. (2014) 38:832-843; Jakate et al., Arch. Pathol. Lab. Med. (2006) 130:362-367; Tison et al., Allergy Clin. Immunol. (2010) Abstract 714), implying that the elevated mast cells in these patients may be responsible for the gastrointestinal symptomatology. As the patients met the same symptom criteria as patients for the anti-Siglec-8 antibody study, and they failed or were not adequately controlled on system organ class (SOC) treatments, they have a substantial need for better treatments.

A reduction in the number or activation of tissue mast cells may be useful in the treatment of patients with moderate to severe gastrointestinal symptoms and increased number of mast cells in the stomach and/or the duodenum, a condition referred to as mast cell gastritis and/or gastroenteritis in this study.

As mast cell gastritis and gastroenteritis are relatively poorly described disease entities, there are no Food and Drug Administration (FDA)-approved treatments. Current therapies and disease management for these patients include a plethora of various approaches, including proton pump inhibitors (PPIs), restricted/elemental diets, systemic or oral corticosteroids, and occasional off-label use of immunomodulatory biologics.

The study described in this example is designed to test the safety and efficacy of anti-Siglec-8 antibody treatment in patients with mast cell gastritis and/or gastroenteritis.

### Dose Selection

In this study, patients with mast cell gastritis and/or gastroenteritis are treated with up to six doses of anti-Siglec-8 HEKA (non-fucosylated IgG1) antibody (also referred to as “study drug”) administered every 4 weeks. The doses are 0.3 mg/kg for the first infusion, 1 mg/kg for the second infusion, and 3 mg/kg for 4 subsequent infusions.

### Patient Selection

The population for this study is adult male and female patients with mast cell gastritis and/or gastroenteritis aged between 18 and 80 years.

Patient inclusion criteria include:

1) Male or female patients aged between 18 and 80 years at the time of signing of the informed consent form (ICF).
2) Failed the screening process for the previous anti-Siglec-8 antibody study due to not meeting the eligibility criterion of eosinophilia of the gastric mucosa (defined as greater than or equal to 30 eosinophils/HPF in 5 HPFs) or duodenal mucosa (defined as greater than or equal to 30 eosinophils/HPF in 3 HPFs) from the esophago-gastro-duodenoscopy (EGD) performed during the anti-Siglec-8 antibody study screening period.
3) Average weekly score of greater than or equal to 3 (on a scale from 0-10) recorded for abdominal pain, diarrhea, or nausea on a patient-reported outcome (PRO) questionnaire during at least 2 weeks out of 3 weeks of PRO collection. A minimum of 4 questionnaires were completed each qualifying week.
4) Had greater than or equal to 30 mast cells/HPF in at least 3 HPFs in the duodenal and/or gastric mucosa from the EGD performed during the previous anti-Siglec-8 antibody study screening period.
5) Subjects fail or are not adequately controlled on standard of care treatments for EG or EGE symptoms (which could include PPIs, systemic or topical corticosteroids, and/or dict, among others).
6) If on other treatments for EG, EGE, or EoE at enrollment, patients had stable dose for at least 5 half-lives prior to screening and were willing to continue on that dose for the duration of the study.
7) If patient were on pre-existing dietary restrictions, they are willing to maintain those dietary restrictions throughout the study, as much as possible.
8) Able and willing to comply with all study procedures.

Patient exclusion criteria include:

1) Known hypersensitivity to any constituent of the study drug.
2) Presence of abnormal laboratory values considered by the Investigator to be clinically significant.
3) Any disease, condition (medical or surgical), or cardiac abnormality, which, in the opinion of the Investigator, would place the subject at increased risk.
4) Known history of alcohol, drug, or other substance abuse or dependence.
5) Participation in a concurrent interventional study with the last intervention occurring within 30 days prior to administration of study drug (or 90 days or 5 half-lives, whichever was longer, for biologic products).
6) Women who are pregnant, breastfeeding, or planning to become pregnant while participating in the study.

### Study Design

This study is a Phase 1b, multicenter, open-label study to evaluate the safety, tolerability, and clinical benefit of anti-Siglec-8 antibody, given as monthly infusions for up to 6 doses in patients with mast cell gastritis and/or gastroenteritis. This study includes patients who were screened and met all patient selection criteria for the previous anti-Siglec-8 antibody study, except for the criterion of having greater than or equal to 30 eosinophils/HPF in 5 HPFs in the gastric mucosa or greater than or equal to 30 eosinophils/HPF in 3 HPFs in the duodenal mucosa. To be eligible for this study, patients have greater than or equal to 30 mast cells/HPF in at least 3 HPFs in the gastric and/or duodenal mucosa. Patients are consented for this study after they failed screening for the previous anti-Siglec-8 antibody study.

The study is designed as follows:

1) A 45-day screening period with baseline evaluations for eligibility. Baseline evaluations from the previous anti-Siglec-8 antibody study can be used as baseline assessments for this study if collected within 45 days of the start of enrollment. Screening biopsy results from the Esophago-Gastro-Duodenoscopy (EGD) from the previous anti-Siglec-8 antibody study can be used if symptoms persisted (as indicated in Inclusion criterion number 3).
2) If patients fail screening for the previous anti-Siglec-8 antibody study due to not having greater than equal to 30 eosinophils/HPF in the gastric or duodenal mucosa, but had greater than or equal to 30 mast cells/HPF in at least 3 HPFs in the duodenal and/or gastric mucosa, they receive 6 doses of anti-Siglec-8 antibody by intravenous infusion on Days 1, 29 (±3 days), 57 (±3 days), 85 (±3 days), 113 (±3 days), and 141 (±3 days) in this study.
3) A repeat EGD with biopsy is performed on Day 155 (+3 days) or approximately 2 weeks after last dose of study drug if patient was terminated early.
4) Pre-study medications and pre-existing dietary restrictions remain unchanged throughout the study. Patients undergo a standardized baseline evaluation of eating habits, food habits/restrictions, and food avoidance behaviors and are asked to maintain similar habits and restrictions throughout the study.

### Primary Objective

The primary objective of this study is to evaluate the safety and tolerability of anti-Siglec-8 antibody in patients with mast cell gastritis and/or gastroenteritis.

### Secondary Objectives

The secondary objectives of this study are to evaluate the effects of anti-Siglec-8 antibody in patients with mast cell gastritis and/or gastroenteritis for the following parameters:

1) Change in number of mast cells/HPF in gastric and duodenal biopsies.
2) Change in gastrointestinal symptom score as estimated by a daily patient-reported outcome (PRO) questionnaire.
3) Change in absolute peripheral blood eosinophil counts.

### Pharmacodynamics Outcome Measures

Blood (serum) is collected for assessment of anti-Siglec-8 antibody concentrations using a validated enzyme-linked immunosorbent assay (ELISA).

### Efficacy Outcome Measures

The number of mast cells in gastric and duodenal mucosa are evaluated. In addition, the number of eosinophils and mast cells in esophageal mucosa are evaluated in patients with concomitant eosinophilic esophagitis.

Other efficacy outcome measures in this study include:

1) Percent change from baseline in the number of mast cells/HPF in gastric and/or duodenal mucosa in patients with mast cell gastritis and/or gastroenteritis.
2) Change from baseline in the weekly averages of gastrointestinal symptomatology as measured by the PRO questionnaire (total and item daily scores, including for the following symptoms: abdominal pain intensity, nausea intensity, vomiting intensity, diarrhea frequency, abdominal cramping intensity, bloating intensity, early satiety intensity, and loss of appetite intensity).
3) Proportion of patients with histologic response, defined as less than 30 mast cells/HPF in gastric and/or duodenal mucosa in patients with mast cell gastritis and/or gastroenteritis.
4) Change from baseline in the number of absolute peripheral blood eosinophil counts.
5) Change from baseline in the number of eosinophils and mast cells/HPF in esophageal biopsies in patients with concomitant eosinophilic esophagitis.
6) Morphological assessment of gastric and duodenal biopsies before and after treatment.
7) Percent change from baseline in body weight.
8) Change from baseline in Patient-Reported Functional Health and Well-being Survey SF-36.

### Study Drug, Dose and Administration

All patients receive 6 intravenous infusions of anti-Siglec-8 antibody during the study, administered as a single peripheral intravenous infusion using an infusion pump as indicated in the study Pharmacy Manual. The exact dose is calculated prior to each infusion and based on patient weight at the time. Anti-Siglec-8 antibody at a dose of 0.3 mg/kg is prepared according to patient's body weight and administered on Day 1. Anti-Siglec-8 antibody at a dose of 1 mg/kg is prepared according to patient's body weight and administered on Day 29 (±3 days). Subsequent infusions of at a dose of 3 mg/kg are prepared according to patient's body weight and administered on Day 57 (±3 days), Day 85 (±3 days), Day 113 (±3 days), and Day 141 (±3 days).

Safety and tolerability are assessed throughout the study by monitoring and evaluating adverse events (AEs), the severity of which is assessed by the NCI Common Terminology Criteria for Adverse Events (CTCAE). All AEs are assigned a severity grade and are assessed to determine whether they are clinically significant and related to the study drug.

### Example 2: Symptomatic Patients Suspected of Eosinophilic Gastritis and/or Enteritis have Elevated Mucosal Mast Cell Counts without Eosinophilia

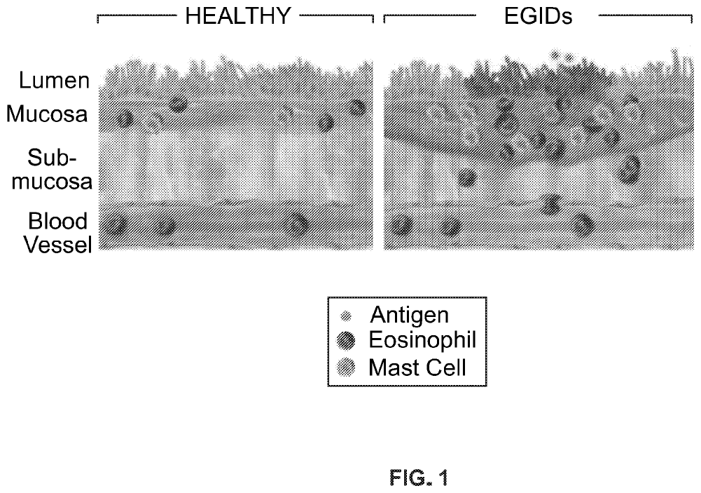

Pathologic accumulation and over-activation of eosinophils are implicated in multiple chronic inflammatory diseases in the GI tract (FIG. 1), including eosinophilic esophagitis (EoE), gastritis (EG), enteritis (EEn), and colitis (collectively termed eosinophilic gastrointestinal diseases, EGIDs). Patients with EGIDs have decreased quality of life due to debilitating symptoms such as dysphagia/difficulty swallowing, abdominal pain, nausea, vomiting, and diarrhea.

While the pathogenesis of EGIDs has historically been thought to be driven by eosinophils, mast cells have also been shown to be elevated in EoE (Caldwell et al. (2014) J. Allergy Clin. Immunol. 134:1114-1124; Youngblood et al. (2019) JCI Insight 4(19)). However, the role of mast cells in EGIDs, particularly other than EoE, has yet to be established. EG and EEn affect 45,000-50,000 patients in the U.S., though this number may be a significant underestimate. Current treatment options such as dietary restriction and corticosteroids have limited efficacy and/or are inappropriate for chronic use. As such, there remains a significant unmet need for novel targeted therapies.

As noted in Example 1, during enrollment for an ongoing study evaluating the efficacy and safety of anti-Siglec-8 antibody for treatment of patients with eosinophilic gastritis and/or gastroenteritis, a sub-population of patients were identified that did not have the pre-requisite number of eosinophils in the gastric and/or duodenal mucosa, but instead had a substantial number of mast cells (in most cases greater than 30 mast cells/high power field (HPF)) in the stomach and/or duodenal mucosa). This Example characterizes these symptomatic patients with suspected EG/EEn who did not meet histopathologic entry criteria for mucosal eosinophilia for the Phase 2 study of anti-Siglec-8 antibody in patients with EG/EEn.

### Screening Protocol

Patients with prior diagnosis or suspected EG/EEn entered screening. Subjects with an average weekly score of ≥3 intensity (0-10 scale) for abdominal pain, diarrhea, and/or nausea for ≥2 weeks on a patient-reported outcome (PRO) questionnaire qualified for an upper endoscopy (EGD) with biopsy.

Multiple biopsies were taken from each symptomatic subject according to a standardized protocol: 8-10 gastric biopsies, 4-6 duodenal biopsies, and 4-6 esophageal biopsies (only if subject had a history of EoE or if EoE features were observed during EGD). Entry criteria were: ≥30 eosinophils (eos)/high-powered field (hpf; area of 0.237 mm²) in 5 hpfs (stomach) and/or ≥30 eos/hpf in 3 hpfs (duodenum); and no other other known cause for GI symptoms or tissue eosinophilia.

Daily PRO questionnaire captured 8 symptoms: abdominal pain, nausea, diarrhea, vomiting, early satiety, loss of appetite, abdominal cramping, and bloating.

### Results

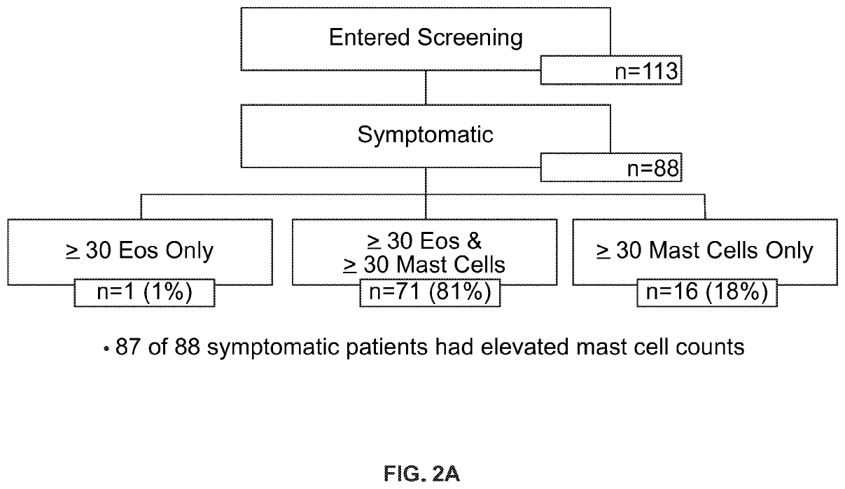

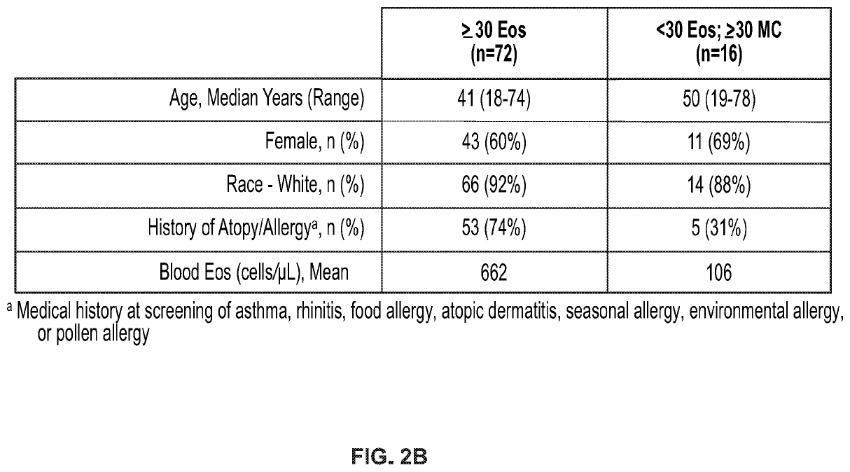

The patient distribution is shown in FIG. 2A. 113 patients entered screening, and 88 were found to be symptomatic. Of these 88, 71 (81%) were found to have ≥30 eos and ≥30 mast cells when screened as described above, 16 (18%) were found to have ≥30 mast cells only, and only 1 (1%) was found to have ≥30 eos only. Thus, 87 out of 88 symptomatic patients had elevated mast cell counts. FIG. 2B compares the baseline characteristics of patients having ≥30 cos (72 in total) with patients having ≥30 mast cells but <30 cos (16 in total).

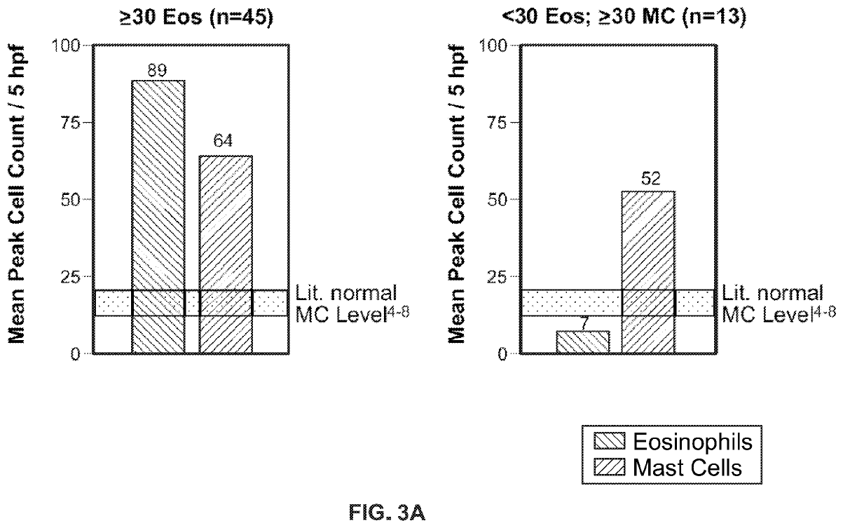

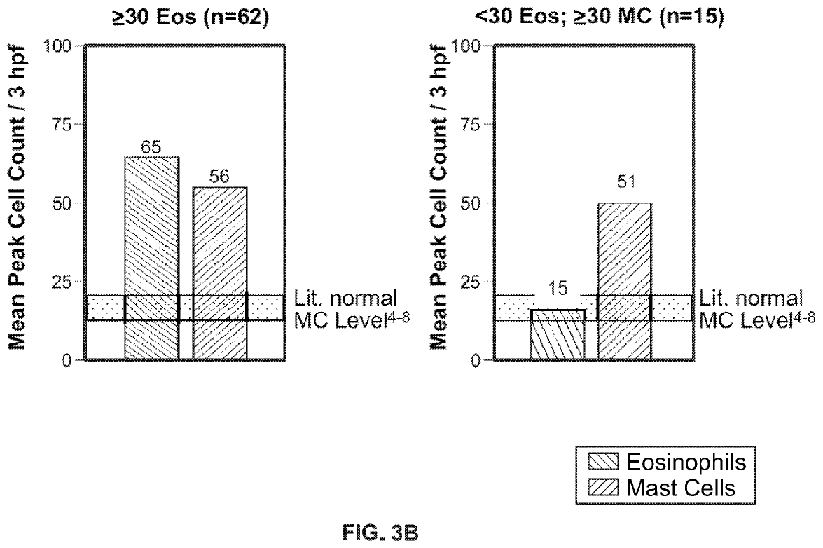

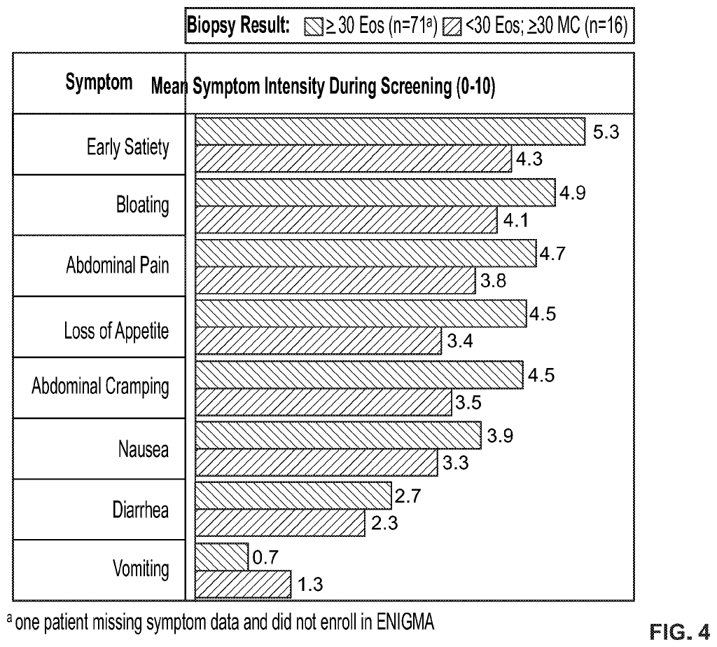

Numbers of eosinophils and mast cells in either stomach (FIG. 3A) or duodenal (FIG. 3B) biopsies are shown in FIGS. 3A and 3B. In stomach biopsies (FIG. 3A), patients with ≥30 eos/hpf in 5 hpfs had a mean peak cell count (per 5 hpf) of 89 eosinophils and 64 mast cells, whereas patients with ≥30 mast cells/hpf in 5 hpfs but <30 cos/hpf in 5 hpfs had a mean peak cell count (per 5 hpf) of 7 eosinophils and 52 mast cells. In duodenal biopsies (FIG. 3B), patients with ≥30 eos/hpf in 3 hpfs had a mean peak cell count (per 3 hpf) of 65 eosinophils and 56 mast cells, whereas patients with ≥30 mast cells/hpf in 3 hpfs but <30 eos/hpf in 3 hpfs had a mean peak cell count (per 3 hpf) of 15 eosinophils and 51 mast cells. FIG. 4 shows the mean symptom intensity score for both patient populations with respect to early satiety, bloating, abdominal pain, loss of appetite, abdominal cramping, nausea, diarrhea, and vomiting (top to bottom). Similar symptom profiles were observed between the two groups (i.e., patients whose biopsies showed ≥30 eos/hpf vs. patients whose biopsies showed <30 eos/hpf but ≥30 mast cells/hpf).

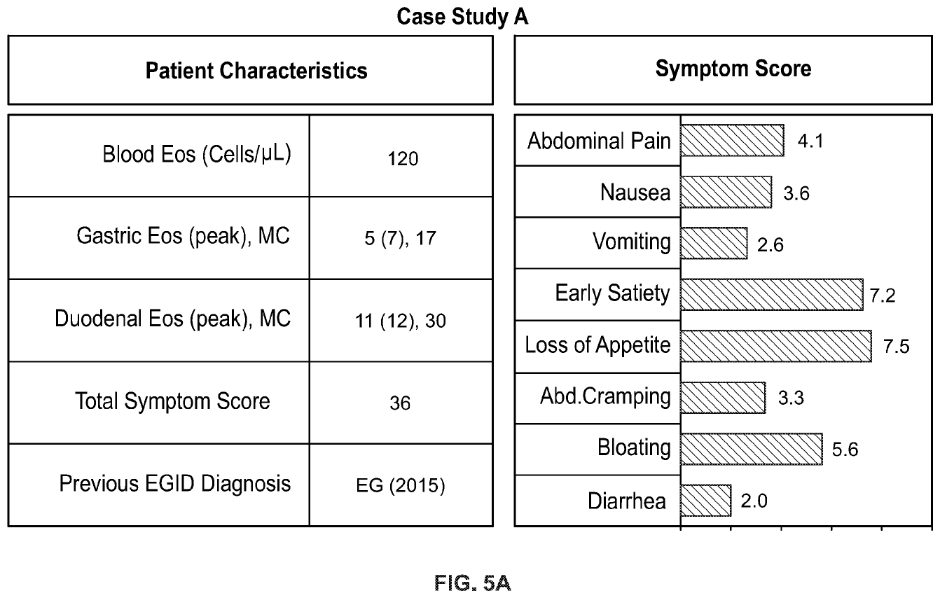

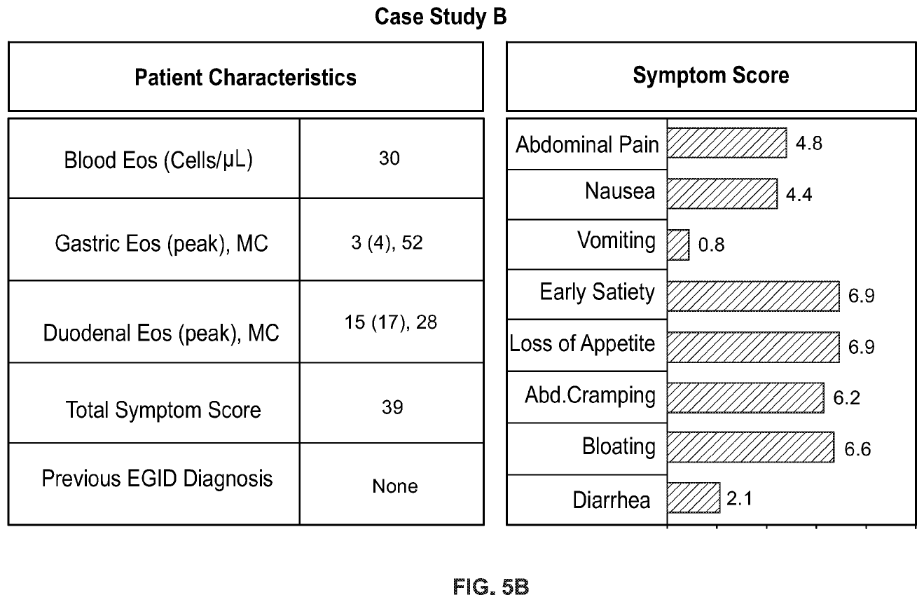

Two individual case studies are shown in FIGS. 5A & 5B. The patient in case study A (FIG. 5A) had been diagnosed with biopsy-confirmed EG in 2015. This patient was not on topical or systemic steroids prior to the screening biopsy. The patient in case study B (FIG. 5B) had no prior EGID diagnosis. These symptoms are consistent with EG and/or EEn despite low eosinophils at screening.

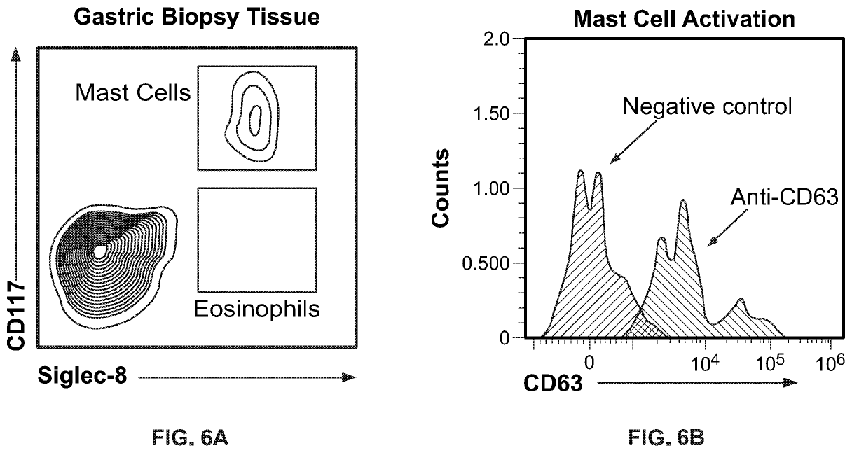

In addition, analysis of human gastric biopsy tissue indicated that increased activation of mast cells was seen in tissues where only mast cells (and not eosinophils) were found to be elevated. Human gastric biopsy tissue was processed into single cells, and mast cells (CD117+ Siglec-8+ cells) and eosinophils (CD117− Siglec-8+ cells) were isolated by flow cytometry (FIG. 6A). From these, mast cells were further analyzed for the mast cell activation and degranulation marker CD63 using flow cytometry (FIG. 6B). To determine if mast cells were activated, cells were stained with anti-CD63 or negative control antibody. These results demonstrate activation of mast cells in these tissues.

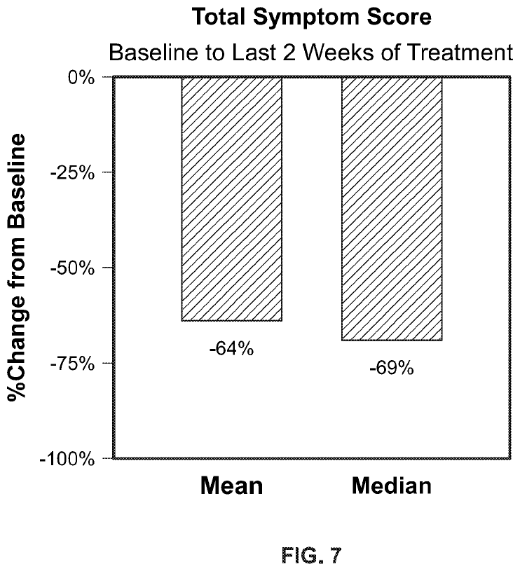

Outcome data from the PRO questionnaire described above were also analyzed. FIG. 7 shows the mean and median change in total symptom score from baseline (daily average of the screening period) to the average daily score for the two weeks after the last dose of anti-Siglec-8 antibody. The results demonstrate a mean 64% and median 69% reduction in total symptom score after the last dose of anti-Siglec-8 antibody, as compared to baseline.

### CONCLUSION

88 patients with suspected EG and/or EEn and active symptoms underwent endoscopy and biopsy. 72/88 met histological eosinophil criteria for the study. 87/88 (99%) patients screened had elevated mast cell counts in gastric and/or duodenal tissue biopsies. Symptom profiles were similar between patients with and without tissue eosinophilia.

These data suggest that mast cells play an important pathogenic role in patients with suspected EG/EEn and raise the possibility of a non-eosinophilic condition driven by mast cells. Due to anti-Siglec-8 antibody's ability to inhibit mast cells, patients with elevated mast cells without tissue eosinophilia were offered to participate in an open-label anti-Siglec-8 antibody clinical trial.

SEQUENCES
All polypeptide sequences are presented N-terminal to
C-terminal unless otherwise noted.
All nucleic acid sequences are presented 5′ to 3′ unless otherwise noted.
Amino acid sequence of mouse 2E2 heavy chain variable domain
(SEQ ID NO: 1)
QVQLKESGPGLVAPSQSLSITCTVSGFSLTIYGAHWVRQPPGRGLEWLGVIWAGGSTNY
NSALMSRLSISKDNSKSQVFLKINSLQTDDTALYYCARDGSSPYYYSMEYWGQGTSVT
VSS
Amino acid sequence of 2E2 RHA heavy chain variable domain
(SEQ ID NO: 2)
EVQLVESGGGLVQPGGSLRLSCAASGFSLTIYGAHWVRQAPGKGLEWVSVIWAGGSTN
YNSALMSRFTISKDNSKNTVYLQMNSLRAEDTAVYYCARDGSSPYYYSMEYWGQGTT
VTVSS
Amino acid sequence of 2E2 RHB heavy chain variable domain
(SEQ ID NO: 3)
EVQLVESGGGLVQPGGSLRLSCAVSGFSLTIYGAHWVRQAPGKGLEWLGVIWAGGSTN
YNSALMSRLSISKDNSKNTVYLQMNSLRAEDTAVYYCARDGSSPYYYSMEYWGQGTT
VTVSS
Amino acid sequence of 2E2 RHC heavy chain variable domain
(SEQ ID NO: 4)
EVQLVESGGGLVQPGGSLRLSCAVSGFSLTIYGAHWVRQAPGRGLEWVSVIWAGGSTN
YNSALMSRFTISKDNSKNTVYLQMNSLRAEDTAVYYCARDGSSPYYYSMEYWGQGTT
VTVSS
Amino acid sequence of 2E2 RHD heavy chain variable domain
(SEQ ID NO: 5)
EVQLVESGGGLVQPGGSLRLSCAASGFSLTIYGAHWVRQAPGKGLEWLSVIWAGGSTN
YNSALMSRFTISKDNSKNTVYLQMNSLRAEDTAVYYCARDGSSPYYYSMEYWGQGTT
VTVSS
Amino acid sequence of 2E2 RHE heavy chain variable domain
(SEQ ID NO: 6)
EVQLVESGGGLVQPGGSLRLSCAASGFSLTIYGAHWVRQAPGKGLEWVGVIWAGGST
NYNSALMSRFTISKDNSKNTVYLQMNSLRAEDTAVYYCARDGSSPYYYSMEYWGQGT
TVTVSS
Amino acid sequence of 2E2 RHF heavy chain variable domain
(SEQ ID NO: 7)
EVQLVESGGGLVQPGGSLRLSCAASGFSLTIYGAHWVRQAPGKGLEWVSVIWAGGSTN
YNSALMSRLTISKDNSKNTVYLQMNSLRAEDTAVYYCARDGSSPYYYSMEYWGQGTT
VTVSS
Amino acid sequence of 2E2 RHG heavy chain variable domain
(SEQ ID NO: 8)
EVQLVESGGGLVQPGGSLRLSCAASGFSLTIYGAHWVRQAPGKGLEWVSVIWAGGSTN
YNSALMSRFSISKDNSKNTVYLQMNSLRAEDTAVYYCARDGSSPYYYSMEYWGQGTT
VTVSS
Amino acid sequence of 2E2 RHA2 heavy chain variable domain
(SEQ ID NO: 9)
QVQLQESGPGLVKPSETLSLTCTVSGGSISIYGAHWIRQPPGKGLEWIGVIWAGGSTNYN
SALMSRVTISVDTSKNQFSLKLSSVTAADTAVYYCARDGSSPYYYSMEYWGQGTLVTV
SS
Amino acid sequence of 2E2 RHB2 heavy chain variable domain
(SEQ ID NO: 10)
QVQLQESGPGLVKPSETLSLTCTVSGFSLTIYGAHWVRQPPGKGLEWLGVIWAGGSTN
YNSALMSRLSISKDNSKNQVSLKLSSVTAADTAVYYCARDGSSPYYYSMEYWGQGTL
VTVSS
Amino acid sequence of 2E2 RHE S-G mutant heavy chain variable domain
(SEQ ID NO: 11)
EVQLVESGGGLVQPGGSLRLSCAASGFSLTIYGAHWVRQAPGKGLEWVGVIWAGGST
NYNSALMSRFTISKDNSKNTVYLQMNSLRAEDTAVYYCARDGSSPYYYGMEYWGQGT
TVTVSS
Amino acid sequence of 2E2 RHE E-D heavy chain variable domain
(SEQ ID NO: 12)
EVQLVESGGGLVQPGGSLRLSCAASGFSLTIYGAHWVRQAPGKGLEWVGVIWAGGST
NYNSALMSRFTISKDNSKNTVYLQMNSLRAEDTAVYYCARDGSSPYYYSMDYWGQGT
TVTVSS
Amino acid sequence of 2E2 RHE Y-V heavy chain variable domain
(SEQ ID NO: 13)
EVQLVESGGGLVQPGGSLRLSCAASGFSLTIYGAHWVRQAPGKGLEWVGVIWAGGST
NYNSALMSRFTISKDNSKNTVYLQMNSLRAEDTAVYYCARDGSSPYYYSMEVWGQGT
TVTVSS
Amino acid sequence of 2E2 RHE triple mutant heavy chain variable domain
(SEQ ID NO: 14)
EVQLVESGGGLVQPGGSLRLSCAASGFSLTIYGAHWVRQAPGKGLEWVGVIWAGGST
NYNSALMSRFTISKDNSKNTVYLQMNSLRAEDTAVYYCARDGSSPYYYGMDVWGQG
TTVTVSS
Amino acid sequence of mouse 2E2 light chain variable domain
(SEQ ID NO: 15)
QIILTQSPAIMSASPGEKVSITCSATSSVSYMHWFQQKPGTSPKLWIYSTSNLASGVPVRF
SGSGSGTSYSLTISRMEAEDAATYYCQQRSSYPFTFGSGTKLEIK
Amino acid sequence of 2E2 RKA light chain variable domain
(SEQ ID NO: 16)
EIVLTQSPATLSLSPGERATLSCSATSSVSYMHWFQQKPGQAPRLLIYSTSNLASGIPARF
SGSGSGTDFTLTISSLEPEDFAVYYCQQRSSYPFTFGPGTKLDIK
Amino acid sequence of 2E2 RKB light chain variable domain
(SEQ ID NO: 17)
EIILTQSPATLSLSPGERATLSCSATSSVSYMHWFQQKPGQAPRLWIYSTSNLASGVPARF
SGSGSGTDYTLTISSLEPEDFAVYYCQQRSSYPFTFGPGTKLDIK
Amino acid sequence of 2E2 RKC light chain variable domain
(SEQ ID NO: 18)
EIILTQSPATLSLSPGERATLSCSATSSVSYMHWFQQKPGQAPRLLIYSTSNLASGIPARFS
GSGSGTDFTLTISSLEPEDFAVYYCQQRSSYPFTFGPGTKLDIK
Amino acid sequence of 2E2 RKD light chain variable domain
(SEQ ID NO: 19)
EIVLTQSPATLSLSPGERATLSCSATSSVSYMHWFQQKPGQAPRLWIYSTSNLASGIPARF
SGSGSGTDFTLTISSLEPEDFAVYYCQQRSSYPFTFGPGTKLDIK
Amino acid sequence of 2E2 RKE light chain variable domain
(SEQ ID NO: 20)
EIVLTQSPATLSLSPGERATLSCSATSSVSYMHWFQQKPGQAPRLLIYSTSNLASGVPAR
FSGSGSGTDFTLTISSLEPEDFAVYYCQQRSSYPFTFGPGTKLDIK
Amino acid sequence of 2E2 RKF light chain variable domain
(SEQ ID NO: 21)
EIVLTQSPATLSLSPGERATLSCSATSSVSYMHWFQQKPGQAPRLLIYSTSNLASGIPARF
SGSGSGTDYTLTISSLEPEDFAVYYCQQRSSYPFTFGPGTKLDIK
Amino acid sequence of 2E2 RKG light chain variable domain
(SEQ ID NO: 22)
EIVLTQSPATLSLSPGERATLSCSATSSVSYMHWYQQKPGQAPRLLIYSTSNLASGIPARF
SGSGSGTDFTLTISSLEPEDFAVYYCQQRSSYPFTFGPGTKLDIK
Amino acid sequence of 2E2 RKA F-Y mutant light chain variable domain
(SEQ ID NO: 23)
EIVLTQSPATLSLSPGERATLSCSATSSVSYMHWFQQKPGQAPRLLIYSTSNLASGIPARF
SGSGSGTDFTLTISSLEPEDFAVYYCQQRSSYPYTFGPGTKLDIK
Amino acid sequence of 2E2 RKF F-Y mutant light chain variable domain
(SEQ ID NO: 24)
EIVLTQSPATLSLSPGERATLSCSATSSVSYMHWFQQKPGQAPRLLIYSTSNLASGIPARF
SGSGSGTDYTLTISSLEPEDFAVYYCQQRSSYPYTFGPGTKLDIK
Amino acid sequence of HEKA heavy chain and HEKF heavy chain
(SEQ ID NO: 75)
EVQLVESGGGLVQPGGSLRLSCAASGFSLTIYGAHWVRQAPGKGLEWVGVIWAGGST
NYNSALMSRFTISKDNSKNTVYLQMNSLRAEDTAVYYCARDGSSPYYYSMEYWGQGT
TVTVSSASTKGPSVFPLAPSSKSTSGGTAALGCLVKDYFPEPVTVSWNSGALTSGVHTFP
AVLQSSGLYSLSSVVTVPSSSLGTQTYICNVNHKPSNTKVDKRVEPKSCDKTHTCPPCPA
PELLGGPSVFLFPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPVLDSDGSFFL
YSKLTVDKSRWQQGNVFSCSVMHEALHNHYTQKSLSLSPG
Amino acid sequence of HEKA light chain
(SEQ ID NO: 76)
EIVLTQSPATLSLSPGERATLSCSATSSVSYMHWFQQKPGQAPRLLIYSTSNLASGIPARF
SGSGSGTDFTLTISSLEPEDFAVYYCQQRSSYPFTFGPGTKLDIKRTVAAPSVFIFPPSDEQ
LKSGTASVVCLLNNFYPREAKVQWKVDNALQSGNSQESVTEQDSKDSTYSLSSTLTLS
KADYEKHKVYACEVTHQGLSSPVTKSFNRGEC
Amino acid sequence of HEKF light chain
(SEQ ID NO: 77)
EIVLTQSPATLSLSPGERATLSCSATSSVSYMHWFQQKPGQAPRLLIYSTSNLASGIPARF
SGSGSGTDYTLTISSLEPEDFAVYYCQQRSSYPFTFGPGTKLDIKRTVAAPSVFIFPPSDEQ
LKSGTASVVCLLNNFYPREAKVQWKYDNALQSGNSQESVTEQDSKDSTYSLSSTLTLS
KADYEKHKVYACEVTHQGLSSPVTKSFNRGEC
Amino acid sequence of IgG1 heavy chain constant region
(SEQ ID NO: 78)
ASTKGPSVFPLAPSSKSTSGGTAALGCLVKDYFPEPVTVSWNSCALTSGVHTFPAVLQSS
GLYSLSSVVTVPSSSLGTQTYICNVNHKPSNTKVDKRVEPKSCDKTHTCPPCPAPELLGG
PSVFLFPPKPKDTLMISRTPEVTCVVVDVSHEDPEVKFNWYVDGVEVHNAKTKPREEQ
YNSTYRVVSVLTVLHQDWLNGKEYKCKVSNKALPAPIEKTISKAKGQPREPQVYTLPPS
REEMTKNQVSLTCLVKGFYPSDIAVEWESNGQPENNYKTTPPVLDSDGSFFLYSKLTYD
KSRWQQGNVFSCSVMHEALHNHYTQKSLSLSPG
Amino acid sequence of IgG4 heavy chain constant region
(SEQ ID NO: 79)
ASTKGPSVFPLAPCSRSTSESTAALGCLVKDYFPEPVTVSWNSGALTSGVHTFPAVLQSS
GLYSLSSVVTVPSSSLGTKTYTCNVDHKPSNTKVDKRVESKYGPPCPPCPAPEFLGGPSV
FLFPPKPKDTLMISRTPEVTCVVVDVSQEDPEVQFNWYVDGVEVHNAKTKPREEQFNST
YRVVSVLTVLHQDWLNGKEYKCKVSNKGLPSSIEKTISKAKGQPREPQVYTLPPSQEEM
TKNQVSLTCLVKGFYPSDIAVEWESNGQPENNYKTTPPVLDSDGSFFLYSRLTVDKSRW
QEGNVFSCSVMHEALHNHYTQKSLSLSLG
Amino acid sequence of Ig kappa light chain constant region
(SEQ ID NO: 80)
RTVAAPSVFIFPPSDEQLKSGTASVVCLLNNFYPREAKVQWKVDNALQSGNSQESVTEQ
DSKDSTYSLSSTLTLSKADYEKHKVYACEVTHQGLSSPVTKSFNRGEC
Amino acid sequence of murine 2C4 and 2E2 IgG1 heavy chain
(SEQ ID NO: 81)
QVQLKRASGPGLVAPSQSLSITCTVSGFSLTIYGAHWVRQPPGKGLEWLGVIWAGGSTN
YNSALMSRLSISKDNSKSQVFLKINSLQTDDTALYYCARDGSSPYYYSMEYWGQGTSV
TVSSAKTTPPSYYPLAPGSAAQTNSMVTLGCLVKGYFPEPVTVTWNSGSLSSGVHTFPA
VLESDLYTLSSSVTVPSSPRPSETVTCNVAHPASSTKVDKKIVPRDCGCKPCICTVPEVSS
VFIFPPKPKDVLTITLTPKVTCVVVDISKDDPEVQFSWFVDDVEVHTAQTQPREEQFNST
FRSVSELPIMHQDWLNGKEFKCRVNSAAFPAPIEKTISKTKGRPKAPQVYTIPPPKEQMA
KDKVSLTCMITDFFPEDITVEWQWNGQPAENYKNTQPIMNTNGSYFVYSKLNVQKSN
WEAGNTFTCSVLHEGLHNHHTEKSLSHSPG
Amino acid sequence of murine 2C4 kappa light chain
(SEQ ID NO: 82)
EIILTQSPAIMSASPGEKVSITCSATSSVSYMHWFQQKPGTSPKLWIYSTSNLASGVPVRF
SGSGSGTSYSLTISRMEAEDAATYYCQQRSSYPFTFGSGTKLEIKADAAPTVSIFPPSSEQ
LTSGGASVVCFLNNFYPKDINVKWKIDGSERQNGVLNSWTDQDSKDSTYSMSSTLTLT
KDEYERHNSYTCEATHKTSTSPIVKSFNRNEC
Amino acid sequence of murine 2E2 kappa light chain
(SEQ ID NO: 83)
QIILTQSPAIMSASPGEKVSITCSATSSVSYMHWFQQKPGTSPKLWIYSTSNLASGVPVRF
SGSGSGTSYSLTISRMEAEDAATYYCQQRSSYPFTFGSGTKLEIKADAAPTVSIFPPSSEQ
LTSGGASVVCFLNNFYPKDINVKWKIDGSERQNGVLNSWTDQDSKDSTYSMSSTLTLT
KDEYERHNSYTCEATHKTSTSPIVKSFNRNEC
Amino acid sequence of chimeric 2C4 and 2E2 IgG1 heavy chain
(SEQ ID NO: 84)
QVQLKRASGPGLVAPSQSLSITCTVSGFSLTIYGAHWVRQPPGKGLEWLGVIWAGGSTN
YNSALMSRLSISKDNSKSQVFLKINSLQTDDTALYYCARDGSSPYYYSMEYWGQGTSV
TVSSASTKGPSVFPLAPSSKSTSGGTAALGCLVKDYFPEPVTVSWNSGALTSGVHTFPAV
LQSSGLYSLSSVVTVPSSSLGTQTYICNVNHKPSNTKVDKRVEPKSCDKTHTCPPCPAPE
LLGGPSVFLFPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAKGQPREPQVY
TLPPSREEMTKNQVSLTCLVKGFYPSDIAVEWESNGQPENNYKTTPPVLDSDGSFFLYS
KLTVDKSRWQQGNVFSCSVMHEALHNHYTQKSLSLSPG
Amino acid sequence of chimeric 2C4 kappa light chain
(SEQ ID NO: 85)
EIILTQSPAIMSASPGEKVSITCSATSSVSYMHWFQQKPGTSPKLWIYSTSNLASGVPVRF
SGSGSGTSYSLTISRMEAEDAATYYCQQRSSYPFTFGSGTKLEIKRTVAAPSVFIFPPSDE
QLKSGTASVVCLLNNFYPREAKVQWKVDNALQSGNSQESVTEQDSKDSTYSLSSTLTL
SKADYEKHKVYACEVTHQGLSSPVTKSFNRGEC
Amino acid sequence of chimeric 2E2 kappa light chain
(SEQ ID NO: 86)
QIILTQSPAIMSASPGEKVSITCSATSSVSYMHWFQQKPGTSPKLWIYSTSNLASGVPVRF
SGSGSGTSYSLTISRMEAEDAATYYCQQRSSYPFTFGSGTKLEIKRTVAAPSVFIFPPSDE
QLKSGTASVVCLLNNFYPREAKVQWKVDNALQSGNSQESVTEQDSKDSTYSLSSTLTL
SKADYEKHKVYACEVTHQGLSSPVTKSFNRGEC
Amino acid sequence of HEKA IgG4 heavy chain (IgG4 contains a S228P
mutation)
(SEQ ID NO: 87)
EVQLVESGGGLVQPGGSLRLSCAASGFSLTIYGAHWVRQAPGKGLEWVGVIWAGGST
NYNSALMSRFTISKDNSKNTVYLQMNSLRAEDTAVYYCARDGSSPYYYSMEYWGQGT
TVTVSSASTKGPSVFPLAPCSRSTSESTAALGCLVKDYFPEPVTVSWNSGALTSGVHTFP
AVLQSSGLYSLSSVVTVPSSSLGTKTYTCNVDHKPSNTKVDKRVESKYGPPCPPCPAPEF
LGGPSVFLFPPKPKDTLMISRTPEVTCVVVDVSQEDPEVQFNWYVDGVEVHNAKTKPR
EEQFNSTYRVVSVLTVLHQDWLNGKEYKCKVSNKGLPSSIEKTISKAKGQPREPQVYTL
PPSQEEMTKNQVSLTCLVKGFYPSDIAVEWESNGQPENNYKTTPPVLDSDGSFFLYSRL
TVDKSRWQEGNVFSCSVMHEALHNHYTQKSLSLSLG
Amino acid sequence of mouse 1C3 heavy chain variable domain (underlined
residues comprise CDRs H1 and H2 according to Chothia numbering)
(SEQ ID NO: 106)
EVQVVESGGDLVKSGGSLKLSCAASGFPFSSYAMSWVRQTPDKRLEWVAIISSGGSYTY
YSDSVKGRFTISRDNAKNTLYLQMSSLKSEDTAMYYCARHETAQAAWFAYWGQGTLV
TVSA
Amino acid sequence of mouse 1H10 heavy chain variable domain (underlined
residues comprise CDRs H1 and H2 according to Chothia numbering)
(SEQ ID NO: 107)
EVQLQQSGAELVRPGASVKLSCTASGFNIKDYYMYWVKQRPEQGLEWIGRIAPEDGDT
EYAPKFQGKATVTADTSSNTAYLHLSSLTSEDTAVYYCTTEGNYYGSSILDYWGQGTT
LTVSS
Amino acid sequence of mouse 4F11 heavy chain variable domain (underlined
residues comprise CDRs H1 and H2 according to Chothia numbering)
(SEQ ID NO: 108)
QVQLQQSGAELVKPGASVKISCKASGYAFRSSWMNWVKQRPGKGLEWIGQIYPGDDY
TNYNGKFKGKVTLTADRSSSTAYMQLSSLTSEDSAVYFCARLGPYGPFADWGQGTLVT
VSA
Amino acid sequence of mouse 1C3 light chain variable domain
(SEQ ID NO: 109)
QIVLTQSPAIMSASPGEKVTMTCSASSSVSYMHWYQQKSGTSPKRWIYDTSKLAYGVP
ARFSGSGSGTSYSLTISSMEAEDAATYYCQQWSSNPPTFGGGTKLEIK
Amino acid sequence of mouse 1H10 light chain variable domain
(SEQ ID NO: 110)
DIQMTQTTSSLSASLGDRVTISCRASQDITNYLNWYQQKPDGTVKLLIYFTSRLHSGVPS
RFSGSGSGTDYSLTISNLEQEDIATYFCQQGNTLPWTFGGGTKLEIK
Amino acid sequence of mouse 4F11 light chain variable domain
(SEQ ID NO: 111)
QIVLTQSPAIVSASPGEKVTMTCSASSSVSYMYWYQQRPGSSPRLLIYDTSSLASGVPVR
FSGSGSGTSYSLTISRIESEDAANYYCQQWNSDPYTFGGGTKLEIK
Amino acid sequence of human Siglec-8 Domain 1
(SEQ ID NO: 112)
MEGDRQYGDGYLLQVQELVTVQEGLCVHVPCSFSYPQDGWTDSDPVHGYWFRAGDR
PYQDAPVATNNPDREVQAETQGRFQLLGDIWSNDCSLSIRDARKRDKGSYFFRLERGS
MKWSYKSQLNYKTKQLSVFVTALTHRP
Amino acid sequence of human Siglec-8 Domain 2
(SEQ ID NO: 113)
DILILGTLESGHSRNLTCSVPWACKQGTPPMISWIGASVSSPGPTTARSSVLTLTPKPQPH
GTSLTCQVTLPGTGVTTTSTVRLDVS
Amino acid sequence of human Siglec-8 Domain 3
(SEQ ID NO: 114)
YPPWNLTMTVFQGDATASTALGNGSSLSVLEGQSLRLVCAVNSNPPARLSWTRGSLTL
CPSRSSNPGLLELPRVHVRDEGEFTCRAQNAQGSQHISLSLSLQNEGTGTSRPVSQVTLA
AVGG
Amino acid sequence of human Siglec-8 Domain 1 Fusion Protein
(SEQ ID NO: 115)
MEGDRQYGDGYLLQVQELVTVQEGLCVHVPCSFSYPQDGWTDSDPVHGYWFRAGDR
PYQDAPVATNNPDREVQAETQGRFQLLGDIWSNDCSLSIRDARKRDKGSYFFRLERGS
MKWSYKSQLNYKTKQLSVFVTALTHRPIEGRSDKTHTCPPCPAPELLGGPSVFLFPPKPK
DTLMISRTPEVTCVVVDVSHEDPEVKFNWYVDGVEVHNAKTKPREEQYNSTYRVVSV
LTVLHQDWLNGKEYKCKVSNKALPAPIEKTISKAKGQPREPQVYTLPPSREEMTKNQVS
LTCLVKGFYPSDIAVEWESNGQPENNYKTTPPVLDSDGSFFLYSKLTVDKSRWQQGNV
FSCSVMHEALHNHYTQKSLSLSPGK
Amino acid sequence of human Siglec-8 Domains 1 and 2 Fusion Protein
(SEQ ID NO: 116)
MEGDRQYGDGYLLQVQELVTVQEGLCVHVPCSFSYPQDGWTDSDPVHGYWFRAGDR
PYQDAPVATNNPDREVQAETQGRFQLLGDIWSNDCSLSIRDARKRDKGSYFFRLERGS
MKWSYKSQLNYKTKQLSVFVTALTHRPDILILGTLESGHSRNLTCSVPWACKQGTPPMI
SWIGASVSSPGPTTARSSVLTLTPKPQDHGTSLTCQVTLPGTGVTTTSTVRLDVSIEGRSD
KTHTCPPCPAPELLGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALPAPIEKTISK
AKGQPREPQVYTLPPSREEMTKNQVSLTCLVKGFYPSDIAVEWESNGQPENNYKTTPPV
LDSDGSFFLYSKLTVDKSRWQQGNVFSCSVMHEALHNHYTQKSLSLSPGK
Amino acid sequence of human Siglec-8 Domains 1, 2, and 3 Fusion Protein
(SEQ ID NO: 117)
MEGDRQYGDGYLLQVQELVTVQEGLCVHVPCSFSYPQDGWTDSDPVHGYWFRAGDR
PYQDAPVATNNPDREVQAETQGRFQLLGDIWSNDCSLSIRDARKRDKGSYFFRLERGS
MKWSYKSQLNYKTKQLSVFVTALTHRPDILILGTLESGHSRNLTCSVPWACKQGTPPMI
SWIGASVSSPGPTTARSSVLTLTPKPQDHGTSLTCQVTLPGTGVTTTSTVRLDVSYPPWN
LTMTVFQGDATASTALGNGSSLSVLEGQSLRLVCAVNSNPPARLSWTRGSLTLCPSRSS
NPGLLELPRVHVRDEGEFTCRAQNAQGSQHISLSLSLQNEGTGTSRPVSQVTLAAVGGIE
GRSDKTHTCPPCPAPELLGGPSVFLFPPKPKDTLMISRTPEVTCVVVDYSHEDPEVKFNW
YVDGVEVHNAKTKPREEQYNSTYRVVSVLTVLHQDWLNGKEYKCKVSNKALPAPIEK
TISKAKGQPREPQVYTLPPSRDELTKNQVSLTCLVKGFYPSDIAVEWESNGQPENNYKT
TPPVLDSDGSFFLYSKLTVDKSRWQQGNVFSCSVMHEALHNHYTQKSLSLSPGK

What is claimed is:

1. A method for treating or preventing one or more symptoms of gastritis, enteritis, duodenitis, or gastroenteritis in an individual, comprising:
(a) detecting number of mast cells from a first sample obtained from gastric, duodenal, jejunal, ileal, or colonic mucosa of the individual;
(b) detecting number of eosinophils from a second sample obtained from gastric, duodenal, jejunal, ileal, or colonic mucosa of the individual; and
(c) if the first sample has an increased number of mast cells as compared to a mast cell reference and the second sample does not have increased number of eosinophils as compared to an eosinophil reference, administering to the individual an effective amount of a composition comprising an antibody that binds to human Siglec-8.

2. A method for treating or preventing one or more symptoms of gastritis, enteritis, duodenitis, or gastroenteritis in an individual comprising administering to the individual an effective amount of a composition comprising an antibody that binds to human Siglec-8, wherein the individual has an increased number of mast cells in at least a portion of the gastric, duodenal, jejunal, ileal, or colonic mucosa as compared to a mast cell reference, and wherein the individual does not have increased number of eosinophils in at least a portion of the gastric, duodenal, jejunal, ileal, or colonic mucosa as compared to an eosinophil reference.

3. The method of claim 2, wherein a first sample obtained from the gastric, duodenal, jejunal, ileal, or colonic mucosa of the individual has an increased number of mast cells as compared to the mast cell reference, and wherein a second sample obtained from the gastric, duodenal, jejunal, ileal, or colonic mucosa of the individual does not have increased number of eosinophils as compared to the eosinophil reference.

4. The method of claim 1 or claim 3, wherein the first and the second samples are the same.

5. The method of any one of claims 1, 3, and 4, wherein one or both of the first and second samples is/are from a gastric or duodenal biopsy.

6. The method of any one of claims 1, 3, and 4, wherein one or both of the first and second samples is/are from an esophago-gastro-duodenoscopy (EGD) with biopsy.

7. The method of any one of claims 1, 3, and 4-6, wherein the first sample has at least one high-power field (HPF) that has a mast cell count of 20 or more mast cells per HPF.

8. The method of claim 7, wherein mast cells are detected by immunohistochemical (IHC) staining for tryptase, CD117, or IgE receptor.

9. The method of any one of claims 1, 3, and 4-8, wherein the second sample has one or more HPFs that each have an eosinophil count of less than 30 eosinophils per HPF.

10. The method of any one of claims 1, 3, and 4-8, wherein the second sample is obtained from the gastric mucosa of the individual, and wherein the second sample does not have at least five HPFs that each have an eosinophil count of 30 or more eosinophils per HPF.

11. The method of any one of claims 1, 3, and 4-8, wherein the second sample is obtained from the duodenal mucosa of the individual, and wherein the second sample does not have at least three HPFs that each have an eosinophil count of 30 or more eosinophils per HPF.

12. The method of any one of claims 1, 3, and 4-11, wherein the number of mast cells in the first sample is detected 45 days or less prior to administration of the composition.

13. The method of any one of claims 1-12, wherein the individual has, or has been diagnosed with, gastroesophageal reflux disease (GERD).

14. The method of claim 13, wherein the individual is refractory to treatment with an antacid, H2 blocker, and/or proton pump inhibitor.

15. The method of any one of claims 1-12, wherein the individual has, or has been diagnosed with, irritable bowel syndrome (IBS).

16. The method of any one of claims 1-12, wherein the individual has, or has been diagnosed with, functional dyspepsia.

17. The method of any one of claims 1-12, wherein the individual has had, or has previously been diagnosed with, eosinophilic gastritis, and wherein the individual has one or more symptoms of eosinophilic gastritis without elevated eosinophils.

18. The method of any one of claims 1-12, wherein the individual has had, or has previously been diagnosed with, eosinophilic gastroenteritis, and wherein the individual has one or more symptoms of eosinophilic gastroenteritis without elevated eosinophils.

19. The method of any one of claims 1-18, wherein one or both of a number or activity of mast cells in a sample obtained from the gastric, duodenal, jejunal, ileal, or colonic mucosa of the individual are reduced after administration of the composition as compared to a baseline level before administration of the composition.

20. The method of any one of claims 1-19, wherein, prior to administration of the composition, the individual has failed or is not adequately controlled by one or more standard-of-care treatments for gastritis or gastroenteritis.

21. The method of claim 20, wherein the one or more standard-of-care treatments for gastritis or gastroenteritis are selected from the group consisting of proton pump inhibitor (PPI) treatment, corticosteroid treatment, and dietary treatment.

22. The method of any one of claims 1-21, wherein the one or more symptom(s) of gastritis, duodenitis, enteritis, or gastroenteritis in the individual are reduced after administration of the composition as compared to a baseline level before administration of the composition.

23. The method of claim 22, wherein the one or more symptom(s) of gastritis, duodenitis, enteritis, or gastroenteritis in the individual are reduced by at least 60% after administration of the composition as compared to a baseline level before administration of the composition.

24. The method of any one of claims 1-21, wherein one or more of abdominal pain, nausea, vomiting, loss of appetite, abdominal cramping, fullness before finishing a meal, bloating, diarrhea, and liquid or watery stools in the individual are reduced after administration of the composition as compared to a baseline level before administration of the composition.

25. A method for treating or preventing one or more symptoms of esophagitis in an individual, comprising:
(a) detecting number of mast cells from a first sample obtained from esophageal mucosa of the individual,
(b) detecting number of eosinophils from a second sample obtained from the esophageal mucosa of the individual, and
(c) if the first sample has an increased number of mast cells as compared to a mast cell reference and the second sample does not have increased number of eosinophils as compared to an eosinophil reference, administering to the individual an effective amount of a composition comprising an antibody that binds to human Siglec-8.

26. A method for treating or preventing one or more symptoms of esophagitis in an individual comprising administering to the individual an effective amount of a composition comprising an antibody that binds to human Siglec-8, wherein the individual has an increased number of mast cells in at least a portion of the esophageal mucosa as compared to a mast cell reference, and wherein the individual does not have increased number of eosinophils in at least a portion of the esophageal mucosa as compared to an eosinophil reference.

27. The method of claim 26, wherein a first sample obtained from the esophageal mucosa of the individual has an increased number of mast cells as compared to the mast cell reference, and wherein a second sample obtained from the esophageal mucosa of the individual does not have increased number of eosinophils as compared to the eosinophil reference.

28. The method of claim 25 or claim 27, wherein the first and the second samples are the same.

29. The method of any one of claims 25, 27, and 28, wherein one or both of the first and second samples is/are from an esophageal biopsy sample.

30. The method of any one of claims 25, 27, and 28, wherein one or both of the first and second samples is/are from an esophago-gastro-duodenoscopy (EGD) with biopsy.

31. The method of any one of claims 25, 27, and 28-30, wherein the first sample has at least one high-power field (HPF) with a mast cell count of 10 or more mast cells per HPF.

32. The method of claim 31, wherein mast cells are detected by immunohistochemical (IHC) staining for tryptase, CD117, or IgE receptor.

33. The method of any one of claims 25, 27, and 28-32, wherein the second sample has one or more HPFs with an eosinophil count of less than 15 eosinophils per HPF.

34. The method of any one of claims 25, 27, and 28-32, wherein the second sample does not have an HPF with an eosinophil count of 15 or more eosinophils per HPF.

35. The method of any one of claims 25-34, wherein the individual has, or has been diagnosed with, gastroesophageal reflux disease (GERD).

36. The method of claim 35, wherein the individual is refractory to treatment with an antacid, H2 blocker, and/or proton pump inhibitor.

37. The method of any one of claims 25-36, wherein one or more of a number, activity, or location of mast cells in a sample obtained from the esophageal mucosa of the individual is reduced after administration of the composition as compared to a baseline level before administration of the composition.

38. The method of any one of claims 25-37, wherein, prior to administration of the composition, the individual has failed or is not adequately controlled by one or more standard-of-care treatments for esophagitis.

39. The method of any one of claims 25-38, wherein the one or more symptom(s) of esophagitis in the individual are reduced after administration of the composition as compared to a baseline level before administration of the composition.

40. The method of claim 39, wherein the one or more symptom(s) of esophagitis in the individual are reduced by at least 60% after administration of the composition as compared to a baseline level before administration of the composition.

41. The method of any one of claims 25-38, wherein one or more of heartburn, nausea, dysphagia/difficulty swallowing, vomiting, abdominal pain, cough, food impaction, early satiety, loss of appetite, chest pain, feeding intolerance or refusal, and gastroesophageal reflux in the individual are reduced after administration of the composition as compared to a baseline level before administration of the composition.

42. A method for treating or preventing one or more symptoms of colitis in an individual, comprising:
(a) detecting number of mast cells from a first sample obtained from colonic mucosa of the individual;
(b) detecting number of eosinophils from a second sample obtained from colonic mucosa of the individual; and
(c) if the first sample has an increased number of mast cells as compared to a mast cell reference and the second sample does not have increased number of eosinophils as compared to an eosinophil reference, administering to the individual an effective amount of a composition comprising an antibody that binds to human Siglec-8.

43. A method for treating or preventing one or more symptoms of colitis in an individual comprising administering to the individual an effective amount of a composition comprising an antibody that binds to human Siglec-8, wherein the individual has an increased number of mast cells in at least a portion of the colonic mucosa as compared to a mast cell reference, and wherein the individual does not have increased number of eosinophils in at least a portion of the colonic mucosa as compared to an eosinophil reference.

44. The method of claim 43, wherein a first sample obtained from the colonic mucosa of the individual has an increased number of mast cells as compared to the mast cell reference, and wherein a second sample obtained from the colonic mucosa of the individual does not have increased number of eosinophils as compared to the eosinophil reference.

45. The method of claim 42 or claim 44, wherein the first and the second samples are the same.

46. The method of any one of claims 42-45, wherein the colitis is ulcerative colitis.

47. The method of any one of claims 42-46, wherein the first sample has at least one high-power field (HPF) with a mast cell count of 30 or more mast cells per HPF.

48. The method of claim 47, wherein mast cells are detected by immunohistochemical (IHC) staining for tryptase, CD117, or IgE receptor.

49. The method of any one of claims 42-48, wherein the second sample has one or more HPFs with an eosinophil count of less than 60 eosinophils per HPF.

50. The method of any one of claims 42-48, wherein the second sample does not have an HPF with an eosinophil count of 60 or more eosinophils per HPF.

51. The method of any one of claims 42-50, wherein the number of mast cells in the first sample is detected 45 days or less prior to administration of the composition.

52. The method of any one of claims 42-51, wherein the individual has had, or has previously been diagnosed with, eosinophilic colitis, and wherein the individual has one or more symptoms of eosinophilic colitis without elevated eosinophils.

53. The method of any one of claims 42-52, wherein one or both of a number or activity of mast cells in a sample obtained from the colonic mucosa of the individual are reduced after administration of the composition as compared to a baseline level before administration of the composition.

54. The method of any one of claims 42-53, wherein, prior to administration of the composition, the individual has failed or is not adequately controlled by one or more standard-of-care treatments for colitis.

55. The method of any one of claims 42-54, wherein the one or more symptom(s) of colitis in the individual are reduced after administration of the composition as compared to a baseline level before administration of the composition.

56. The method of claim 55, wherein the one or more symptom(s) of colitis in the individual are reduced by at least 60% after administration of the composition as compared to a baseline level before administration of the composition.

57. The method of any one of claims 1-56, wherein the composition is administered by intravenous infusion.

58. The method of claim 57, wherein the composition is administered by intravenous infusion once a month for 3 or more months.

59. The method of any one of claims 1-56, wherein the composition is administered by subcutaneous injection.

60. The method of any one of claims 1-56, wherein the composition is administered by intravenous infusion at one or more doses comprising between about 0.3 mg/kg and about 3.0 mg/kg of the antibody.

61. The method of any one of claims 1-56, wherein the method comprises administering to the individual a first dose comprising about 0.3 mg/kg of the antibody, a second dose comprising about 1.0 mg/kg of the antibody, and a third dose comprising about 3.0 mg/kg of the antibody.

62. The method of claim 61, wherein the method comprises administering to the individual a first dose comprising about 0.3 mg/kg of the antibody on Day 1, a second dose comprising about 1.0 mg/kg of the antibody between Day 26 and Day 32, a third dose comprising about 3.0 mg/kg of the antibody between Day 54 and Day 60, a fourth dose comprising about 3.0 mg/kg of the antibody between Day 82 and Day 88, a fifth dose comprising about 3.0 mg/kg of the antibody between Day 110 and Day 116, and a sixth dose comprising about 3.0 mg/kg of the antibody between Day 138 and Day 144.

63. The method of any one of claims 1-62, wherein the antibody comprises a Fc region and N-glycoside-linked carbohydrate chains linked to the Fc region, wherein less than 50% of the N-glycoside-linked carbohydrate chains of the antibody in the composition contain a fucose residue.

64. The method of claim 63, wherein substantially none of the N-glycoside-linked carbohydrate chains of the antibody in the composition contain a fucose residue.

65. The method of any one of claims 1-64, wherein the antibody comprises a heavy chain variable region and a light chain variable region, wherein the heavy chain variable region comprises (i) HVR-H1 comprising the amino acid sequence of SEQ ID NO:61, (ii) HVR-H2 comprising the amino acid sequence of SEQ ID NO:62, and (iii) HVR-H3 comprising the amino acid sequence of SEQ ID NO:63; and/or wherein the light chain variable region comprises (i) HVR-L1 comprising the amino acid sequence of SEQ ID NO:64, (ii) HVR-L2 comprising the amino acid sequence of SEQ ID NO:65, and (iii) HVR-L3 comprising the amino acid sequence of SEQ ID NO:66.

66. The method of any one of claims 1-64, wherein the antibody comprises a heavy chain variable region and a light chain variable region, wherein the heavy chain variable region comprises (i) HVR-H1 comprising the amino acid sequence of SEQ ID NO:61, (ii) HVR-H2 comprising the amino acid sequence of SEQ ID NO:62, and (iii) HVR-H3 comprising the amino acid sequence selected from SEQ ID NOs:67-70; and/or wherein the light chain variable region comprises (i) HVR-L1 comprising the amino acid sequence of SEQ ID NO:64, (ii) HVR-L2 comprising the amino acid sequence of SEQ ID NO:65, and (iii) HVR-L3 comprising the amino acid sequence of SEQ ID NO:71.

67. The method of any one of claims 1-64, wherein the antibody comprises a heavy chain variable region comprising the amino acid sequence of SEQ ID NO:6; and/or a light chain variable region comprising an amino acid sequence selected from SEQ ID NO:16 or 21.

68. The method of any one of claims 1-64, wherein the antibody comprises a heavy chain variable region comprising the amino acid sequence selected from SEQ ID NOs:11-14; and/or a light chain variable region comprising an amino acid sequence selected from SEQ ID NOs:23-24.

69. The method of any one of claims 1-64, wherein the antibody comprises a heavy chain variable region comprising the amino acid sequence selected from SEQ ID NOs:2-14; and/or a light chain variable region comprising an amino acid sequence selected from SEQ ID NOs:16-24.

70. The method of any one of claims 1-64, wherein the antibody comprises a heavy chain variable region comprising the amino acid sequence selected from SEQ ID NOs:2-10; and/or a light chain variable region comprising an amino acid sequence selected from SEQ ID NOs:16-22.

71. The method of any one of claims 1-64, wherein the antibody comprises:
(a) heavy chain variable region comprising:
(1) an HC-FR1 comprising the amino acid sequence selected from SEQ ID NOs:26-29;
(2) an HVR-H1 comprising the amino acid sequence of SEQ ID NO:61;
(3) an HC-FR2 comprising the amino acid sequence selected from SEQ ID NOs:31-36;
(4) an HVR-H2 comprising the amino acid sequence of SEQ ID NO:62;
(5) an HC-FR3 comprising the amino acid sequence selected from SEQ ID NOs:38-43;
(6) an HVR-H3 comprising the amino acid sequence of SEQ ID NO:63; and
(7) an HC-FR4 comprising the amino acid sequence selected from SEQ ID NOs:45-46, and/or
(b) a light chain variable region comprising:
(1) an LC-FR1 comprising the amino acid sequence selected from SEQ ID NOs:48-49;
(2) an HVR-L1 comprising the amino acid sequence of SEQ ID NO:64;
(3) an LC-FR2 comprising the amino acid sequence selected from SEQ ID NOs:51-53;
(4) an HVR-L2 comprising the amino acid sequence of SEQ ID NO:65;
(5) an LC-FR3 comprising the amino acid sequence selected from SEQ ID NOs:55-58;
(6) an HVR-L3 comprising the amino acid sequence of SEQ ID NO:66; and
(7) an LC-FR4 comprising the amino acid sequence of SEQ ID NO:60.

72. The method of any one of claims 1-64, wherein the antibody comprises:
(a) heavy chain variable region comprising:
(1) an HC-FR1 comprising the amino acid sequence of SEQ ID NO:26;
(2) an HVR-H1 comprising the amino acid sequence of SEQ ID NO:61;
(3) an HC-FR2 comprising the amino acid sequence of SEQ ID NO:34;
(4) an HVR-H2 comprising the amino acid sequence of SEQ ID NO:62;
(5) an HC-FR3 comprising the amino acid sequence of SEQ ID NO:38;
(6) an HVR-H3 comprising the amino acid sequence of SEQ ID NO:63; and
(7) an HC-FR4 comprising the amino acid sequence of SEQ ID NOs:45; and/or
(b) a light chain variable region comprising:
(1) an LC-FR1 comprising the amino acid sequence of SEQ ID NO:48;
(2) an HVR-L1 comprising the amino acid sequence of SEQ ID NO: 64;
(3) an LC-FR2 comprising the amino acid sequence of SEQ ID NO:51;
(4) an HVR-L2 comprising the amino acid sequence of SEQ ID NO:65;
(5) an LC-FR3 comprising the amino acid sequence of SEQ ID NO:55;
(6) an HVR-L3 comprising the amino acid sequence of SEQ ID NO:66; and
(7) an LC-FR4 comprising the amino acid sequence of SEQ ID NO:60.

73. The method of any one of claims 1-64, wherein the antibody comprises:
(a) heavy chain variable region comprising:
(1) an HC-FR1 comprising the amino acid sequence of SEQ ID NO:26;
(2) an HVR-H1 comprising the amino acid sequence of SEQ ID NO:61;
(3) an HC-FR2 comprising the amino acid sequence of SEQ ID NO:34;
(4) an HVR-H2 comprising the amino acid sequence of SEQ ID NO:62;
(5) an HC-FR3 comprising the amino acid sequence of SEQ ID NO:38;
(6) an HVR-H3 comprising the amino acid sequence of SEQ ID NO:63; and
(7) an HC-FR4 comprising the amino acid sequence of SEQ ID NOs:45; and/or
(b) a light chain variable region comprising:
(1) an LC-FR1 comprising the amino acid sequence of SEQ ID NO:48;
(2) an HVR-L1 comprising the amino acid sequence of SEQ ID NO:64;
(3) an LC-FR2 comprising the amino acid sequence of SEQ ID NO:51;
(4) an HVR-L2 comprising the amino acid sequence of SEQ ID NO:65;
(5) an LC-FR3 comprising the amino acid sequence of SEQ ID NO:58;
(6) an HVR-L3 comprising the amino acid sequence of SEQ ID NO:66; and
(7) an LC-FR4 comprising the amino acid sequence of SEQ ID NO:60.

74. The method of any one of claims 1-64, wherein the antibody comprises:
a heavy chain variable region comprising (i) HVR-H1 comprising the amino acid sequence of SEQ ID NO:88, (ii) HVR-H2 comprising the amino acid sequence of SEQ ID NO:91, and (iii) HVR-H3 comprising the amino acid sequence of SEQ ID NO:94; and/or a light chain variable region comprising (i) HVR-L1 comprising the amino acid sequence of SEQ ID NO:97, (ii) HVR-L2 comprising the amino acid sequence of SEQ ID NO:100, and (iii) HVR-L3 comprising the amino acid sequence of SEQ ID NO:103;
a heavy chain variable region comprising (i) HVR-H1 comprising the amino acid sequence of SEQ ID NO:89, (ii) HVR-H2 comprising the amino acid sequence of SEQ ID NO:92, and (iii) HVR-H3 comprising the amino acid sequence of SEQ ID NO:95; and/or a light chain variable region comprising (i) HVR-L1 comprising the amino acid sequence of SEQ ID NO:98, (ii) HVR-L2 comprising the amino acid sequence of SEQ ID NO:101, and (iii) HVR-L3 comprising the amino acid sequence of SEQ ID NO:104; or
a heavy chain variable region comprising (i) HVR-H1 comprising the amino acid sequence of SEQ ID NO:90, (ii) HVR-H2 comprising the amino acid sequence of SEQ ID NO:93, and (iii) HVR-H3 comprising the amino acid sequence of SEQ ID NO:96; and/or a light chain variable region comprising (i) HVR-L1 comprising the amino acid sequence of SEQ ID NO:99, (ii) HVR-L2 comprising the amino acid sequence of SEQ ID NO:102, and (iii) HVR-L3 comprising the amino acid sequence of SEQ ID NO:105.

75. The method of any one of claims 1-64, wherein the antibody comprises:
a heavy chain variable region comprising the amino acid sequence of SEQ ID NO:106; and/or a light chain variable region comprising the amino acid sequence of SEQ ID NO:109;
a heavy chain variable region comprising the amino acid sequence of SEQ ID NO:107; and/or a light chain variable region comprising the amino acid sequence of SEQ ID NO:110; or
a heavy chain variable region comprising the amino acid sequence of SEQ ID NO:108; and/or a light chain variable region comprising the amino acid sequence of SEQ ID NO:111.

76. The method of any one of claims 1-64, wherein the antibody binds to a human Siglec-8 and a non-human primate Siglec-8.

77. The method of claim 76, wherein the non-human primate is a baboon.

78. The method of claim 76, wherein the antibody binds to an epitope in Domain 1 of human Siglec-8, wherein Domain 1 comprises the amino acid sequence of SEQ ID NO:112.

79. The method of claim 76, wherein the antibody binds to an epitope in Domain 3 of human Siglec-8, wherein Domain 3 comprises the amino acid sequence of SEQ ID NO:114.

80. The method of claim 76, wherein the antibody binds to the same epitope as antibody 4F11.

81. The method of any one of claims 1-64, wherein the antibody binds to an epitope in Domain 2 or Domain 3 of human Siglec-8.

82. The method of claim 81, wherein Domain 2 comprises the amino acid sequence of SEQ ID NO:113.

83. The method of claim 81, wherein the antibody binds to the same epitope as antibody 1C3.

84. The method of claim 81, wherein Domain 3 comprises the amino acid sequence of SEQ ID NO:114.

85. The method of claim 81, wherein the antibody binds to the same epitope as antibody 1H10.

86. The method of any one of claims 1-64, wherein the antibody binds to an epitope in Domain 1 of human Siglec-8 and competes with antibody 4F11 for binding to Siglec-8.

87. The method of claim 86, wherein the antibody does not compete with antibody 2E2 for binding to Siglec-8.

88. The method of claim 87, wherein the antibody is not antibody 2E2.

89. The method of claim 86, wherein Domain 1 comprises the amino acid sequence of SEQ ID NO:112.

90. The method of any one of claims 65-89, wherein the antibody is a human antibody, a humanized antibody, or a chimeric antibody.

91. The method of any one of claims 65-90, wherein the antibody depletes blood eosinophils and inhibits mast cell activation.

92. The method of any one of claims 65-91, wherein the antibody comprises a heavy chain Fc region comprising a human IgG Fc region.

93. The method of claim 92, wherein the human IgG Fc region comprises a human IgG1 Fc region.

94. The method of claim 93, wherein the human IgG1 Fc region is non-fucosylated.

95. The method of claim 92, wherein the human IgG Fc region comprises a human IgG4 Fc region.

96. The method of claim 95, wherein the human IgG4 Fc region comprises the amino acid substitution S228P, wherein the amino acid residues are numbered according to the EU index as in Kabat.

97. The method of any one of claims 65-89, wherein the antibody has been engineered to improve antibody-dependent cell-mediated cytotoxicity (ADCC) activity.

98. The method of claim 97, wherein the antibody comprises at least one amino acid substitution in the Fc region that improves ADCC activity.

99. The method of any one of claims 65-91, wherein at least one or two of the heavy chains of the antibody is non-fucosylated.

100. The method of any one of claims 1-64, wherein the antibody comprises a heavy chain comprising the amino acid sequence of SEQ ID NO:75; and/or a light chain comprising the amino acid sequence selected from SEQ ID NO:76 or 77.

101. The method of any one of claims 1-100, wherein the antibody is a monoclonal antibody.

102. The method of any one of claims 1-101, wherein the composition is administered in combination with one or more additional therapeutic agent(s) for treating or preventing gastritis, gastroenteritis, or esophagitis.

103. The method of claim 102, wherein the one or more additional therapeutic agent(s) for treating or preventing gastritis, gastroenteritis, or esophagitis are selected from the group consisting of PPIs, systemic corticosteroids, topical corticosteroids, antihistamines, mast cell stabilizers, H-2 blockers, anti-IgE antibodies, calcineurin inhibitors, immunomodulatory agents, and immunosuppressive agents.

104. The method of any one of claims 1-103, wherein the individual is a human.

105. The method of any one of claims 1-104, wherein the composition is a pharmaceutical composition comprising the antibody and a pharmaceutically acceptable carrier.

106. An article of manufacture comprising a medicament comprising a composition comprising an antibody that binds to human Siglec-8 and a package insert comprising instructions for administration of the medicament in an individual in need thereof according to any one of claims 1-105.


SEQUENCE LISTING (117 sequences)

SEQ ID NO: 1 - protein, 120 aa, Mus musculus
QVQLKESGPGLVAPSQSLSITCTVSGFSLTIYGAHWVRQPPGKGLEWLGVIWAGGSTNYNSALMSRLSISKDNSKSQVFLKINSLQTDDTALYYCARDGSSPYYYSMEYWGQGTSVTVSS

SEQ ID NO: 2 - protein, 120 aa, Artificial Sequence
  Synthetic Construct
EVQLVESGGGLVQPGGSLRLSCAASGFSLTIYGAHWVRQAPGKGLEWVSVIWAGGSTNYNSALMSRFTISKDNSKNTVYLQMNSLRAEDTAVYYCARDGSSPYYYSMEYWGQGTTVTVSS

SEQ ID NO: 3 - protein, 120 aa, Artificial Sequence
  Synthetic Construct
EVQLVESGGGLVQPGGSLRLSCAVSGFSLTIYGAHWVRQAPGKGLEWLGVIWAGGSTNYNSALMSRLSISKDNSKNTVYLQMNSLRAEDTAVYYCARDGSSPYYYSMEYWGQGTTVTVSS

SEQ ID NO: 4 - protein, 120 aa, Artificial Sequence
  Synthetic Construct
EVQLVESGGGLVQPGGSLRLSCAVSGFSLTIYGAHWVRQAPGKGLEWVSVIWAGGSTNYNSALMSRFTISKDNSKNTVYLQMNSLRAEDTAVYYCARDGSSPYYYSMEYWGQGTTVTVSS

SEQ ID NO: 5 - protein, 120 aa, Artificial Sequence
  Synthetic Construct
EVQLVESGGGLVQPGGSLRLSCAASGFSLTIYGAHWVRQAPGKGLEWLSVIWAGGSTNYNSALMSRFTISKDNSKNTVYLQMNSLRAEDTAVYYCARDGSSPYYYSMEYWGQGTTVTVSS

SEQ ID NO: 6 - protein, 120 aa, Artificial Sequence
  Synthetic Construct
EVQLVESGGGLVQPGGSLRLSCAASGFSLTIYGAHWVRQAPGKGLEWVGVIWAGGSTNYNSALMSRFTISKDNSKNTVYLQMNSLRAEDTAVYYCARDGSSPYYYSMEYWGQGTTVTVSS

SEQ ID NO: 7 - protein, 120 aa, Artificial Sequence
  Synthetic Construct
EVQLVESGGGLVQPGGSLRLSCAASGFSLTIYGAHWVRQAPGKGLEWVSVIWAGGSTNYNSALMSRLTISKDNSKNTVYLQMNSLRAEDTAVYYCARDGSSPYYYSMEYWGQGTTVTVSS

SEQ ID NO: 8 - protein, 120 aa, Artificial Sequence
  Synthetic Construct
EVQLVESGGGLVQPGGSLRLSCAASGFSLTIYGAHWVRQAPGKGLEWVSVIWAGGSTNYNSALMSRFSISKDNSKNTVYLQMNSLRAEDTAVYYCARDGSSPYYYSMEYWGQGTTVTVSS

SEQ ID NO: 9 - protein, 120 aa, Artificial Sequence
  Synthetic Construct
QVQLQESGPGLVKPSETLSLTCTVSGGSISIYGAHWIRQPPGKGLEWIGVIWAGGSTNYNSALMSRVTISVDTSKNQFSLKLSSVTAADTAVYYCARDGSSPYYYSMEYWGQGTLVTVSS

SEQ ID NO: 10 - protein, 120 aa, Artificial Sequence
  Synthetic Construct
QVQLQESGPGLVKPSETLSLTCTVSGFSLTIYGAHWVRQPPGKGLEWLGVIWAGGSTNYNSALMSRLSISKDNSKNQVSLKLSSVTAADTAVYYCARDGSSPYYYSMEYWGQGTLVTVSS

SEQ ID NO: 11 - protein, 120 aa, Artificial Sequence
  Synthetic Construct
EVQLVESGGGLVQPGGSLRLSCAASGFSLTIYGAHWVRQAPGKGLEWVGVIWAGGSTNYNSALMSRFTISKDNSKNTVYLQMNSLRAEDTAVYYCARDGSSPYYYGMEYWGQGTTVTVSS

SEQ ID NO: 12 - protein, 120 aa, Artificial Sequence
  Synthetic Construct
EVQLVESGGGLVQPGGSLRLSCAASGFSLTIYGAHWVRQAPGKGLEWVGVIWAGGSTNYNSALMSRFTISKDNSKNTVYLQMNSLRAEDTAVYYCARDGSSPYYYSMDYWGQGTTVTVSS

SEQ ID NO: 13 - protein, 120 aa, Artificial Sequence
  Synthetic Construct
EVQLVESGGGLVQPGGSLRLSCAASGFSLTIYGAHWVRQAPGKGLEWVGVIWAGGSTNYNSALMSRFTISKDNSKNTVYLQMNSLRAEDTAVYYCARDGSSPYYYSMEVWGQGTTVTVSS

SEQ ID NO: 14 - protein, 120 aa, Artificial Sequence
  Synthetic Construct
EVQLVESGGGLVQPGGSLRLSCAASGFSLTIYGAHWVRQAPGKGLEWVGVIWAGGSTNYNSALMSRFTISKDNSKNTVYLQMNSLRAEDTAVYYCARDGSSPYYYGMDVWGQGTTVTVSS

SEQ ID NO: 15 - protein, 106 aa, Mus musculus
QIILTQSPAIMSASPGEKVSITCSATSSVSYMHWFQQKPGTSPKLWIYSTSNLASGVPVRFSGSGSGTSYSLTISRMEAEDAATYYCQQRSSYPFTFGSGTKLEIK

SEQ ID NO: 16 - protein, 106 aa, Artificial Sequence
  Synthetic Construct
EIVLTQSPATLSLSPGERATLSCSATSSVSYMHWFQQKPGQAPRLLIYSTSNLASGIPARFSGSGSGTDFTLTISSLEPEDFAVYYCQQRSSYPFTFGPGTKLDIK

SEQ ID NO: 17 - protein, 106 aa, Artificial Sequence
  Synthetic Construct
EIILTQSPATLSLSPGERATLSCSATSSVSYMHWFQQKPGQAPRLWIYSTSNLASGVPARFSGSGSGTDYTLTISSLEPEDFAVYYCQQRSSYPFTFGPGTKLDIK

SEQ ID NO: 18 - protein, 106 aa, Artificial Sequence
  Synthetic Construct
EIILTQSPATLSLSPGERATLSCSATSSVSYMHWFQQKPGQAPRLLIYSTSNLASGIPARFSGSGSGTDFTLTISSLEPEDFAVYYCQQRSSYPFTFGPGTKLDIK

SEQ ID NO: 19 - protein, 106 aa, Artificial Sequence
  Synthetic Construct
EIVLTQSPATLSLSPGERATLSCSATSSVSYMHWFQQKPGQAPRLWIYSTSNLASGIPARFSGSGSGTDFTLTISSLEPEDFAVYYCQQRSSYPFTFGPGTKLDIK

SEQ ID NO: 20 - protein, 106 aa, Artificial Sequence
  Synthetic Construct
EIVLTQSPATLSLSPGERATLSCSATSSVSYMHWFQQKPGQAPRLLIYSTSNLASGVPARFSGSGSGTDFTLTISSLEPEDFAVYYCQQRSSYPFTFGPGTKLDIK

SEQ ID NO: 21 - protein, 106 aa, Artificial Sequence
  Synthetic Construct
EIVLTQSPATLSLSPGERATLSCSATSSVSYMHWFQQKPGQAPRLLIYSTSNLASGIPARFSGSGSGTDYTLTISSLEPEDFAVYYCQQRSSYPFTFGPGTKLDIK

SEQ ID NO: 22 - protein, 106 aa, Artificial Sequence
  Synthetic Construct
EIVLTQSPATLSLSPGERATLSCSATSSVSYMHWYQQKPGQAPRLLIYSTSNLASGIPARFSGSGSGTDFTLTISSLEPEDFAVYYCQQRSSYPFTFGPGTKLDIK

SEQ ID NO: 23 - protein, 106 aa, Artificial Sequence
  Synthetic Construct
EIVLTQSPATLSLSPGERATLSCSATSSVSYMHWFQQKPGQAPRLLIYSTSNLASGIPARFSGSGSGTDFTLTISSLEPEDFAVYYCQQRSSYPYTFGPGTKLDIK

SEQ ID NO: 24 - protein, 106 aa, Artificial Sequence
  Synthetic Construct
EIVLTQSPATLSLSPGERATLSCSATSSVSYMHWFQQKPGQAPRLLIYSTSNLASGIPARFSGSGSGTDYTLTISSLEPEDFAVYYCQQRSSYPYTFGPGTKLDIK

SEQ ID NO: 25 - protein, 30 aa, Artificial Sequence
  Synthetic Construct
QVQLKESGPGLVAPSQSLSITCTVSGFSLT

SEQ ID NO: 26 - protein, 30 aa, Artificial Sequence
  Synthetic Construct
EVQLVESGGGLVQPGGSLRLSCAASGFSLT

SEQ ID NO: 27 - protein, 30 aa, Artificial Sequence
  Synthetic Construct
EVQLVESGGGLVQPGGSLRLSCAVSGFSLT

SEQ ID NO: 28 - protein, 30 aa, Artificial Sequence
  Synthetic Construct
QVQLQESGPGLVKPSETLSLTCTVSGGSIS

SEQ ID NO: 29 - protein, 30 aa, Artificial Sequence
  Synthetic Construct
QVQLQESGPGLVKPSETLSLTCTVSGFSLT

SEQ ID NO: 30 - protein, 14 aa, Artificial Sequence
  Synthetic Construct
WVRQPPGKGLEWLG

SEQ ID NO: 31 - protein, 14 aa, Artificial Sequence
  Synthetic Construct
WVRQAPGKGLEWVS

SEQ ID NO: 32 - protein, 14 aa, Artificial Sequence
  Synthetic Construct
WVRQAPGKGLEWLG

SEQ ID NO: 33 - protein, 14 aa, Artificial Sequence
  Synthetic Construct
WVRQAPGKGLEWLS

SEQ ID NO: 34 - protein, 14 aa, Artificial Sequence
  Synthetic Construct
WVRQAPGKGLEWVG

SEQ ID NO: 35 - protein, 14 aa, Artificial Sequence
  Synthetic Construct
WIRQPPGKGLEWIG

SEQ ID NO: 36 - protein, 14 aa, Artificial Sequence
  Synthetic Construct
WVRQPPGKGLEWLG

SEQ ID NO: 37 - protein, 32 aa, Artificial Sequence
  Synthetic Construct
RLSISKDNSKSQVFLKINSLQTDDTALYYCAR

SEQ ID NO: 38 - protein, 32 aa, Artificial Sequence
  Synthetic Construct
RFTISKDNSKNTVYLQMNSLRAEDTAVYYCAR

SEQ ID NO: 39 - protein, 32 aa, Artificial Sequence
  Synthetic Construct
RLSISKDNSKNTVYLQMNSLRAEDTAVYYCAR

SEQ ID NO: 40 - protein, 32 aa, Artificial Sequence
  Synthetic Construct
RLTISKDNSKNTVYLQMNSLRAEDTAVYYCAR

SEQ ID NO: 41 - protein, 32 aa, Artificial Sequence
  Synthetic Construct
RFSISKDNSKNTVYLQMNSLRAEDTAVYYCAR

SEQ ID NO: 42 - protein, 32 aa, Artificial Sequence
  Synthetic Construct
RVTISVDTSKNQFSLKLSSVTAADTAVYYCAR

SEQ ID NO: 43 - protein, 32 aa, Artificial Sequence
  Synthetic Construct
RLSISKDNSKNQVSLKLSSVTAADTAVYYCAR

SEQ ID NO: 44 - protein, 11 aa, Artificial Sequence
  Synthetic Construct
WGQGTSVTVSS

SEQ ID NO: 45 - protein, 11 aa, Artificial Sequence
  Synthetic Construct
WGQGTTVTVSS

SEQ ID NO: 46 - protein, 11 aa, Artificial Sequence
  Synthetic Construct
WGQGTLVTVSS

SEQ ID NO: 47 - protein, 23 aa, Artificial Sequence
  Synthetic Construct
QIILTQSPAIMSASPGEKVSITC

SEQ ID NO: 48 - protein, 23 aa, Artificial Sequence
  Synthetic Construct
EIVLTQSPATLSLSPGERATLSC

SEQ ID NO: 49 - protein, 23 aa, Artificial Sequence
  Synthetic Construct
EIILTQSPATLSLSPGERATLSC

SEQ ID NO: 50 - protein, 15 aa, Artificial Sequence
  Synthetic Construct
WFQQKPGTSPKLWIY

SEQ ID NO: 51 - protein, 15 aa, Artificial Sequence
  Synthetic Construct
WFQQKPGQAPRLLIY

SEQ ID NO: 52 - protein, 15 aa, Artificial Sequence
  Synthetic Construct
WFQQKPGQAPRLWIY

SEQ ID NO: 53 - protein, 15 aa, Artificial Sequence
  Synthetic Construct
WYQQKPGQAPRLLIY

SEQ ID NO: 54 - protein, 32 aa, Artificial Sequence
  Synthetic Construct
GVPVRFSGSGSGTSYSLTISRMEAEDAATYYC

SEQ ID NO: 55 - protein, 32 aa, Artificial Sequence
  Synthetic Construct
GIPARFSGSGSGTDFTLTISSLEPEDFAVYYC

SEQ ID NO: 56 - protein, 32 aa, Artificial Sequence
  Synthetic Construct
GVPARFSGSGSGTDYTLTISSLEPEDFAVYYC

SEQ ID NO: 57 - protein, 32 aa, Artificial Sequence
  Synthetic Construct
GVPARFSGSGSGTDFTLTISSLEPEDFAVYYC

SEQ ID NO: 58 - protein, 32 aa, Artificial Sequence
  Synthetic Construct
GIPARFSGSGSGTDYTLTISSLEPEDFAVYYC

SEQ ID NO: 59 - protein, 10 aa, Artificial Sequence
  Synthetic Construct
FGSGTKLEIK

SEQ ID NO: 60 - protein, 10 aa, Artificial Sequence
  Synthetic Construct
FGPGTKLDIK

SEQ ID NO: 61 - protein, 5 aa, Artificial Sequence
  Synthetic Construct
IYGAH

SEQ ID NO: 62 - protein, 16 aa, Artificial Sequence
  Synthetic Construct
VIWAGGSTNYNSALMS

SEQ ID NO: 63 - protein, 12 aa, Artificial Sequence
  Synthetic Construct
DGSSPYYYSMEY

SEQ ID NO: 64 - protein, 10 aa, Artificial Sequence
  Synthetic Construct
SATSSVSYMH

SEQ ID NO: 65 - protein, 7 aa, Artificial Sequence
  Synthetic Construct
STSNLAS

SEQ ID NO: 66 - protein, 9 aa, Artificial Sequence
  Synthetic Construct
QQRSSYPFT

SEQ ID NO: 67 - protein, 12 aa, Artificial Sequence
  Synthetic Construct
DGSSPYYYGMEY

SEQ ID NO: 68 - protein, 12 aa, Artificial Sequence
  Synthetic Construct
DGSSPYYYSMDY

SEQ ID NO: 69 - protein, 12 aa, Artificial Sequence
  Synthetic Construct
DGSSPYYYSMEV

SEQ ID NO: 70 - protein, 12 aa, Artificial Sequence
  Synthetic Construct
DGSSPYYYGMDV

SEQ ID NO: 71 - protein, 9 aa, Artificial Sequence
  Synthetic Construct
QQRSSYPYT

SEQ ID NO: 72 - protein, 474 aa, Homo sapiens
GYLLQVQELVTVQEGLCVHVPCSFSYPQDGWTDSDPVHGYWFRAGDRPYQDAPVATNNPDREVQAETQGRFQLLGDIWSNDCSLSIRDARKRDKGSYFFRLERGSMKWSYKSQLNYKTKQLSVFVTALTHRPDILILGTLESGHSRNLTCSVPWACKQGTPPMISWIGASVSSPGPTTARSSVLTLTPKPQDHGTSLTCQVTLPGTGVTTTSTVRLDVSYPPWNLTMTVFQGDATASTALGNGSSLSVLEGQSLRLVCAVNSNPPARLSWTRGSLTLCPSRSSNPGLLELPRVHVRDEGEFTCRAQNAQGSQHISLSLSLQNEGTGTSRPVSQVTLAAVGGAGATALAFLSFCIIFIIVRSCRKKSARPAAGVGDTGMEDAKAIRGSASQGPLTESWKDGNPLKKPPPAVAPSSGEEGELHYATLSFHKVKPQDPQGQEATDSEYSEIKIHKRETAETQACLRNHNPSSKEVRG

SEQ ID NO: 73 - protein, 474 aa, Homo sapiens
GYLLQVQELVTVQEGLCVHVPCSFSYPQDGWTDSDPVHGYWFRAGDRPYQDAPVATNNPDREVQAETQGRFQLLGDIWSNDCSLSIRDARKRDKGSYFFRLERGSMKWSYKSQLNYKTKQLSVFVTALTHRPDILILGTLESGHPRNLTCSVPWACKQGTPPMISWIGASVSSPGPTTARSSVLTLTPKPQDHGTSLTCQVTLPGTGVTTTSTVRLDVSYPPWNLTMTVFQGDATASTALGNGSSLSVLEGQSLRLVCAVNSNPPARLSWTRGSLTLCPSRSSNPGLLELPRVHVRDEGEFTCRAQNAQGSQHISLSLSLQNEGTGTSRPVSQVTLAAVGGAGATALAFLSFCIIFIIVRSCRKKSARPAAGVGDTGMEDAKAIRGSASQGPLTESWKDGNPLKKPPPAVAPSSGEEGELHYATLSFHKVKPQDPQGQEATDSEYSEIKIHKRETAETQACLRNHNPSSKEVRG

SEQ ID NO: 74 - protein, 573 aa, Artificial Sequence
  Synthetic Construct
GYLLQVQELVTVQEGLCVHVPCSFSYPQDGWTDSDPVHGYWFRAGDRPYQDAPVATNNPDREVQAETQGRFQLLGDIWSNDCSLSIRDARKRDKGSYFFRLERGSMKWSYKSQLNYKTKQLSVFVTALTHRPDILILGTLESGHSRNLTCSVPWACKQGTPPMISWIGASVSSPGPTTARSSVLTLTPKPQDHGTSLTCQVTLPGTGVTTTSTVRLDVSYPPWNLTMTVFQGDATASTALGNGSSLSVLEGQSLRLVCAVNSNPPARLSWTRGSLTLCPSRSSNPGLLELPRVHVRDEGEFTCRAQNAQGSQHISLSLSLQNEGTGTSRPVSQVTLAAVGGIEGRSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 75 - protein, 449 aa, Artificial Sequence
  Synthetic Construct
EVQLVESGGGLVQPGGSLRLSCAASGFSLTIYGAHWVRQAPGKGLEWVGVIWAGGSTNYNSALMSRFTISKDNSKNTVYLQMNSLRAEDTAVYYCARDGSSPYYYSMEYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 76 - protein, 213 aa, Artificial Sequence
  Synthetic Construct
EIVLTQSPATLSLSPGERATLSCSATSSVSYMHWFQQKPGQAPRLLIYSTSNLASGIPARFSGSGSGTDFTLTISSLEPEDFAVYYCQQRSSYPFTFGPGTKLDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 77 - protein, 213 aa, Artificial Sequence
  Synthetic Construct
EIVLTQSPATLSLSPGERATLSCSATSSVSYMHWFQQKPGQAPRLLIYSTSNLASGIPARFSGSGSGTDYTLTISSLEPEDFAVYYCQQRSSYPFTFGPGTKLDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 78 - protein, 329 aa, Artificial Sequence
  Synthetic Construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 79 - protein, 326 aa, Artificial Sequence
  Synthetic Construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 80 - protein, 107 aa, Artificial Sequence [sequence omitted]

SEQ ID NO: 81 - protein, 444 aa, Mus musculus
QVQLKRASGPGLVAPSQSLSITCTVSGFSLTIYGAHWVRQPPGKGLEWLGVIWAGGSTNYNSALMSRLSISKDNSKSQVFLKINSLQTDDTALYYCARDGSSPYYYSMEYWGQGTSVTVSSAKTTPPSVYPLAPGSAAQTNSMVTLGCLVKGYFPEPVTVTWNSGSLSSGVHTFPAVLESDLYTLSSSVTVPSSPRPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPG

SEQ ID NO: 82 - protein, 212 aa, Mus musculus
EIILTQSPAIMSASPGEKVSITCSATSSVSYMHWFQQKPGTSPKLWIYSTSNLASGVPVRFSGSGSGTSYSLTISRMEAEDAATYYCQQRSSYPFTFGSGTKLEIKADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 83 - protein, 212 aa, Mus musculus
QIILTQSPAIMSASPGEKVSITCSATSSVSYMHWFQQKPGTSPKLWIYSTSNLASGVPVRFSGSGSGTSYSLTISRMEAEDAATYYCQQRSSYPFTFGSGTKLEIKADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 84 - protein, 450 aa, Artificial Sequence
  Synthetic Construct
QVQLKRASGPGLVAPSQSLSITCTVSGFSLTIYGAHWVRQPPGKGLEWLGVIWAGGSTNYNSALMSRLSISKDNSKSQVFLKINSLQTDDTALYYCARDGSSPYYYSMEYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 85 - protein, 213 aa, Artificial Sequence
  Synthetic Construct
EIILTQSPAIMSASPGEKVSITCSATSSVSYMHWFQQKPGTSPKLWIYSTSNLASGVPVRFSGSGSGTSYSLTISRMEAEDAATYYCQQRSSYPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 86 - protein, 213 aa, Artificial Sequence
  Synthetic Construct
QIILTQSPAIMSASPGEKVSITCSATSSVSYMHWFQQKPGTSPKLWIYSTSNLASGVPVRFSGSGSGTSYSLTISRMEAEDAATYYCQQRSSYPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 87 - protein, 446 aa, Artificial Sequence
  Synthetic Construct
EVQLVESGGGLVQPGGSLRLSCAASGFSLTIYGAHWVRQAPGKGLEWVGVIWAGGSTNYNSALMSRFTISKDNSKNTVYLQMNSLRAEDTAVYYCARDGSSPYYYSMEYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 88 - protein, 5 aa, Artificial Sequence
  Synthetic Construct
SYAMS

SEQ ID NO: 89 - protein, 5 aa, Artificial Sequence
  Synthetic Construct
DYYMY

SEQ ID NO: 90 - protein, 5 aa, Artificial Sequence
  Synthetic Construct
SSWMN

SEQ ID NO: 91 - protein, 17 aa, Artificial Sequence
  Synthetic Construct
IISSGGSYTYYSDSVKG

SEQ ID NO: 92 - protein, 17 aa, Artificial Sequence
  Synthetic Construct
RIAPEDGDTEYAPKFQG

SEQ ID NO: 93 - protein, 17 aa, Artificial Sequence
  Synthetic Construct
QIYPGDDYTNYNGKFKG

SEQ ID NO: 94 - protein, 11 aa, Artificial Sequence
  Synthetic Construct
HETAQAAWFAY

SEQ ID NO: 95 - protein, 12 aa, Artificial Sequence
  Synthetic Construct
EGNYYGSSILDY

SEQ ID NO: 96 - protein, 9 aa, Artificial Sequence
  Synthetic Construct
LGPYGPFAD

SEQ ID NO: 97 - protein, 10 aa, Artificial Sequence
  Synthetic Construct
SASSSVSYMH

SEQ ID NO: 98 - protein, 11 aa, Artificial Sequence
  Synthetic Construct
RASQDITNYLN

SEQ ID NO: 99 - protein, 10 aa, Artificial Sequence
  Synthetic Construct
SASSSVSYMY

SEQ ID NO: 100 - protein, 7 aa, Artificial Sequence
  Synthetic Construct
DTSKLAY

SEQ ID NO: 101 - protein, 7 aa, Artificial Sequence
  Synthetic Construct
FTSRLHS

SEQ ID NO: 102 - protein, 7 aa, Artificial Sequence
  Synthetic Construct
DTSSLAS

SEQ ID NO: 103 - protein, 9 aa, Artificial Sequence
  Synthetic Construct
QQWSSNPPT

SEQ ID NO: 104 - protein, 9 aa, Artificial Sequence
  Synthetic Construct
QQGNTLPWT

SEQ ID NO: 105 - protein, 9 aa, Artificial Sequence
  Synthetic Construct
QQWNSDPYT

SEQ ID NO: 106 - protein, 120 aa, Mus musculus
EVQVVESGGDLVKSGGSLKLSCAASGFPFSSYAMSWVRQTPDKRLEWVAIISSGGSYTYYSDSVKGRFTISRDNAKNTLYLQMSSLKSEDTAMYYCARHETAQAAWFAYWGQGTLVTVSA

SEQ ID NO: 107 - protein, 121 aa, Mus musculus
EVQLQQSGAELVRPGASVKLSCTASGFNIKDYYMYWVKQRPEQGLEWIGRIAPEDGDTEYAPKFQGKATVTADTSSNTAYLHLSSLTSEDTAVYYCTTEGNYYGSSILDYWGQGTTLTVSS

SEQ ID NO: 108 - protein, 118 aa, Mus musculus
QVQLQQSGAELVKPGASVKISCKASGYAFRSSWMNWVKQRPGKGLEWIGQIYPGDDYTNYNGKFKGKVTLTADRSSSTAYMQLSSLTSEDSAVYFCARLGPYGPFADWGQGTLVTVSA

SEQ ID NO: 109 - protein, 106 aa, Mus musculus
QIVLTQSPAIMSASPGEKVTMTCSASSSVSYMHWYQQKSGTSPKRWIYDTSKLAYGVPARFSGSGSGTSYSLTISSMEAEDAATYYCQQWSSNPPTFGGGTKLEIK

SEQ ID NO: 110 - protein, 107 aa, Mus musculus
DIQMTQTTSSLSASLGDRVTISCRASQDITNYLNWYQQKPDGTVKLLIYFTSRLHSGVPSRFSGSGSGTDYSLTISNLEQEDIATYFCQQGNTLPWTFGGGTKLEIK

SEQ ID NO: 111 - protein, 106 aa, Mus musculus
QIVLTQSPAIVSASPGEKVTMTCSASSSVSYMYWYQQRPGSSPRLLIYDTSSLASGVPVRFSGSGSGTSYSLTISRIESEDAANYYCQQWNSDPYTFGGGTKLEIK

SEQ ID NO: 112 - protein, 141 aa, Homo sapiens
MEGDRQYGDGYLLQVQELVTVQEGLCVHVPCSFSYPQDGWTDSDPVHGYWFRAGDRPYQDAPVATNNPDREVQAETQGRFQLLGDIWSNDCSLSIRDARKRDKGSYFFRLERGSMKWSYKSQLNYKTKQLSVFVTALTHRP

SEQ ID NO: 113 - protein, 87 aa, Homo sapiens
DILILGTLESGHSRNLTCSVPWACKQGTPPMISWIGASVSSPGPTTARSSVLTLTPKPQDHGTSLTCQVTLPGTGVTTTSTVRLDVS

SEQ ID NO: 114 - protein, 122 aa, Homo sapiens
YPPWNLTMTVFQGDATASTALGNGSSLSVLEGQSLRLVCAVNSNPPARLSWTRGSLTLCPSRSSNPGLLELPRVHVRDEGEFTCRAQNAQGSQHISLSLSLQNEGTGTSRPVSQVTLAAVGG

SEQ ID NO: 115 - protein, 373 aa, Homo sapiens
MEGDRQYGDGYLLQVQELVTVQEGLCVHVPCSFSYPQDGWTDSDPVHGYWFRAGDRPYQDAPVATNNPDREVQAETQGRFQLLGDIWSNDCSLSIRDARKRDKGSYFFRLERGSMKWSYKSQLNYKTKQLSVFVTALTHRPIEGRSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 116 - protein, 460 aa, Homo sapiens
MEGDRQYGDGYLLQVQELVTVQEGLCVHVPCSFSYPQDGWTDSDPVHGYWFRAGDRPYQDAPVATNNPDREVQAETQGRFQLLGDIWSNDCSLSIRDARKRDKGSYFFRLERGSMKWSYKSQLNYKTKQLSVFVTALTHRPDILILGTLESGHSRNLTCSVPWACKQGTPPMISWIGASVSSPGPTTARSSVLTLTPKPQDHGTSLTCQVTLPGTGVTTTSTVRLDVSIEGRSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 117 - protein, 582 aa, Homo sapiens
MEGDRQYGDGYLLQVQELVTVQEGLCVHVPCSFSYPQDGWTDSDPVHGYWFRAGDRPYQDAPVATNNPDREVQAETQGRFQLLGDIWSNDCSLSIRDARKRDKGSYFFRLERGSMKWSYKSQLNYKTKQLSVFVTALTHRPDILILGTLESGHSRNLTCSVPWACKQGTPPMISWIGASVSSPGPTTARSSVLTLTPKPQDHGTSLTCQVTLPGTGVTTTSTVRLDVSYPPWNLTMTVFQGDATASTALGNGSSLSVLEGQSLRLVCAVNSNPPARLSWTRGSLTLCPSRSSNPGLLELPRVHVRDEGEFTCRAQNAQGSQHISLSLSLQNEGTGTSRPVSQVTLAAVGGIEGRSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK